(19) United States
(12) Patent Application Publication
(10) Pub. No.: US 2022/0233299 A1
(43) Pub. Date: Jul. 28, 2022

(54) CELL ENCAPSULATION DEVICES WITH CONTROLLED OXYGEN DIFFUSION DISTANCES

(71) Applicants: W. L. Gore & Associates, Inc., Newark, DE (US); Viacyte, Inc., San Diego, CA (US)

(72) Inventors: Timothy M. Bruhn, Newark, DE (US); Kevin D'Amour, San Diego, CA (US); Christopher Folk, San Diego, CA (US); Edward Gunzel, Newark, DE (US); Evert Kroon, San Diego, CA (US); Laura Martinson, San Diego, CA (US); Craig McGreevy, San Diego, CA (US); Scott Ritrovato, Newark, DE (US); Greg Rusch, Newark, DE (US); Michael Scott, San Diego, CA (US); Lauren R. Zambotti, Newark, DE (US); Qiang (John) Zhang, San Diego, CA (US); Joseph Kakkassery, San Diego, CA (US)

(21) Appl. No.: 17/595,915

(22) PCT Filed: May 30, 2020
(86) PCT No.: PCT/US2020/035452

§ 371 Date: Nov. 29, 2021

Related U.S. Application Data
(60) Provisional application No. 62/855,676, filed on May 31, 2019.

(51) Int. Cl. A61F 2/02; A61L 27/50; A61L 27/16; B01D 71/36; C08L 27/18

(52) U.S. Cl. CPC A61F 2/022; A61L 27/50; A61L 27/16; B01D 71/36; B01D 2325/20; A61F 2210/0076; A61F 2250/0023; A61F 2250/0036; A61F 2250/0041; C08L 27/18

(57) ABSTRACT

Cell encapsulation devices for biological entities and/or cell populations that contain at least one biocompatible membrane composite are provided. The cell encapsulation devices mitigate or tailor the foreign body response from a host such that sufficient blood vessels are able to form at a cell impermeable surface. Additionally, the encapsulation devices have an oxygen diffusion distance that is sufficient for the survival of the encapsulated cells so that the cells are able to secrete a therapeutically useful substance. The biocompatible membrane composite is formed of a cell impermeable layer and a mitigation layer. The cell encapsulation device maintains an optimal oxygen diffusion distance through the design of the cell encapsulation device or through the use of lumen control mechanisms. Lumen control mechanisms include a reinforcing component that is also a nutrient impermeable layer, internal structural pillars, internal tensioning member(s), and/or an internal cell displacing core.

### FIELD

The present invention relates to the field of implantable medical devices and, in particular, to cell encapsulation devices that have a controlled oxygen diffusion distance and uses thereof.

### BACKGROUND

Biological therapies are increasingly viable methods for treating peripheral artery disease, aneurysm, heart disease, Alzheimer's and Parkinson's diseases, autism, blindness, diabetes, and other pathologies.

With respect to biological therapies in general, cells, viruses, viral vectors, bacteria, proteins, antibodies, and other bioactive entities may be introduced into a patient by surgical or other interventional methods that place the bioactive moiety into a tissue bed of a patient. The bioactive entities may be first placed in a device that is then inserted into the patient. Alternatively, the device may be first inserted into the patient with bioactive entities added later.

To maintain a viable and productive population of bioactive entities (e.g., cells), the bioactive entities must maintain access to nutrients, such as oxygen, which are delivered through the blood vessels of the host. To maximize the viability and productivity of the implanted, encapsulated cells, it is necessary to maximize access to the source of oxygen and nutrients by ensuring that the formation of blood vessels be as close as possible to the cells such that the diffusion distance and time needed for transport of the oxygen and nutrients to the implanted, encapsulated cells is minimized.

The implantation of external devices, such as, for example, cell encapsulation devices, into a body triggers an immune response in which foreign body giant cells form and at least partially encapsulate the implanted device. The presence of foreign body giant cells at or near the surface of the implanted cell encapsulation device makes it difficult, if not impossible for blood vessels to form in close proximity to the encapsulated cells, thereby restricting access to the oxygen and nutrients needed to maintain the viability and health of the encapsulated cells.

There remains a need in the art for a cell encapsulation device that provides the implanted cells sufficient immune isolation from the host's immune cells while mitigating or tailoring the foreign body response such that sufficient blood vessels are able to form at a cell impermeable surface. There is also a need for device that provides an optimal oxygen diffusion distance so that the blood vessels at the interface maximizes the ability for the implanted cells to survive and secrete a therapeutically useful substance.

### SUMMARY

In one Aspect (“Aspect 1”), an encapsulation device includes (1) a first biocompatible membrane composite sealed along a portion of its periphery to a second biocompatible membrane composite along a portion of its periphery to define at least one lumen therein and (2) at least one filling tube in fluid communication with the lumen, where at least one of the first biocompatible membrane composite and the second biocompatible membrane composite includes a first layer and a second layer having solid features with a majority of solid feature spacing less than about 50 microns, where the encapsulation device has a majority oxygen diffusion distance of less than 300 microns.

According to another Aspect (“Aspect 2”) further to Aspect 1, the first layer has a mass per area (MpA) less than about 5 g/m².

According to another Aspect (“Aspect 3”) further to Aspect 1 or Aspect 2, the first layer has an MPS (maximum pore size) less than about 1 micron.

According to another Aspect (“Aspect 4”) further to any one of Aspects 1 to 3, the at least one of the first biocompatible membrane composite and the second biocompatible membrane composite has a maximum tensile load in the weakest axis greater than 40 N/m.

According to another Aspect (“Aspect 5”) further to any one of Aspects 1 to 4, the first layer has a first porosity greater than about 50%.

According to another Aspect (“Aspect 6”) further to any one of Aspects 1 to 5, the second layer has a second porosity greater than about 60%.

According to another Aspect (“Aspect 7”) further to any one of Aspects 1 to 6, the second layer has a thickness less than about 200 microns.

According to another Aspect (“Aspect 8”) further to any one of Aspects 1 to 7, the solid features of the second layer each include a representative minor axis, a representative major axis, and a solid feature depth where a majority of at least two of the representative minor axis, where the representative major axis, and the solid feature depth of the second layer is greater than about 5 microns.

According to another Aspect (“Aspect 9”) further to any one of Aspects 1 to 8, the second layer has a pore size from about 1 micron to about 9 microns in effective diameter.

According to another Aspect (“Aspect 10”) further to any one of Aspects 1 to 9, the solid features are connected by fibrils and the fibrils are deformable.

According to another Aspect (“Aspect 11”) further to any one of Aspects 1 to 10, at least a portion of the first solid features in contact with the first layer are bonded solid features.

According to another Aspect (“Aspect 12”) further to any one of Aspects 1 to 11, a majority of the bonded features has a solid feature size from about 3 microns to about 20 microns.

According to another Aspect (“Aspect 13”) further to any one of Aspects 1 to 12, the first layer and the second layer are intimately bonded.

According to another Aspect (“Aspect 14”) further to any one of Aspects 1 to 13, at least one of the first layer and the second layer includes a polymer, a fluoropolymer membrane, a non-fluoropolymer membrane, a woven textile, a non-woven textile, a woven or non-woven collections of fibers or yarns, a fibrous matrix, a spunbound non-woven material, and combinations thereof.

According to another Aspect (“Aspect 15”) further to any one of Aspects 1 to 14, at least one of the first layer and the second layer is a polymer selected from an expanded polytetrafluoroethylene (ePTFE) membrane, a fluorinated ethylene propylene (FEP) membrane and a modified ePTFE membrane.

According to another Aspect (“Aspect 16”) further to any one of Aspects 1 to 15, at least one of the first layer and the second layer is an expanded polytetrafluoroethylene membrane.

According to another Aspect (“Aspect 17”) further to any one of Aspects 1 to 16, the second layer includes at least one of a textile and a non-fluoropolymer membrane.

According to another Aspect (“Aspect 18”) further to Aspect 17, the textile is selected from woven textiles, non-woven textiles, spunbound materials, melt blown fibrous materials, and electrospun nanofibers.

According to another Aspect (“Aspect 19”) further to Aspect 17, the non-fluoropolymer membrane is selected from polyvinylidene difluoride, nanofibers, polysulfones, polyethersulfones, polyarlysulfones, polyether ether ketone, polyethylenes, polypropylenes, polyimides and combinations thereof.

According to another Aspect (“Aspect 20”) further to any one of Aspects 1 to Aspect 19, the second layer includes expanded polytetrafluoroethylene.

According to another Aspect (“Aspect 21”) further to any one of Aspects 1 to 20, the second layer includes nodes, and where the nodes are the solid features.

According to another Aspect (“Aspect 22”) further to any one of Aspects 1 to 21, including a reinforcing component.

According to another Aspect (“Aspect 23”) further to Aspect 22, the reinforcing component is an external reinforcing component.

According to another Aspect (“Aspect 24”) further to Aspect 23, the external reinforcing component has a stiffness from about 0.01 N/cm to about 3 N/cm.

According to another Aspect (“Aspect 25”) further to Aspect 23 or Aspect 24, the external reinforcing component includes a spunbound polyester non-woven material.

According to another Aspect (“Aspect 26”) further to any one of Aspects 23 to 25, the external reinforcing component is a polyester woven mesh.

According to another Aspect (“Aspect 27”) further to Aspect 22, wherein the reinforcing component is an internal reinforcing component.

According to another Aspect, (“Aspect 28”) further to Aspect 27 including an internal reinforcing component.

According to another Aspect (“Aspect 29”) further to Aspect 27 or Aspect 28, the internal reinforcing component is a cell and nutrient impermeable layer.

According to another Aspect (“Aspect 30”) further to any one of Aspects 27 to 29, the internal reinforcing component is substantially centrally located within the encapsulation device and divides the lumen substantially in half.

According to another Aspect (“Aspect 31”) further to any one of Aspects 27 to 30, the internal reinforcing component has thereon structural pillars.

According to another Aspect (“Aspect 32”) further to any one of Aspects 1 to 31, including point bonds between the first biocompatible membrane composite and second biocompatible membrane composite.

According to another Aspect (“Aspect 33”) further to any one of Aspects 1 to 32, including point bonds between a reinforcing component and at least one of the first biocompatible membrane composite and the second biocompatible membrane composite.

According to another Aspect (“Aspect 34”) further to any one of Aspects 1 to 33, including point bonds of approximately 1 mm diameter and spaced from about 0.5 mm to about 9 mm from each other.

According to another Aspect (“Aspect 35”) further to any one of Aspects 1 to 34, including a cell displacing core disposed in the lumen.

According to another Aspect (“Aspect 36”) further to any one of Aspects 1 to 35, including polymeric structural spacers interconnecting the first biocompatible membrane composite to the second biocompatible membrane composite.

According to another Aspect (“Aspect 37”) further to any one of Aspects 1 to 36, the encapsulation device is formed with one or more of a lap seam, a butt seam or a fin seam.

According to another Aspect (“Aspect 38”) further to any one of Aspects 1 to 37, including structural spacers located within the lumen to maintain a desired thickness of the lumen.

According to another Aspect (“Aspect 39”) further to any one of Aspects 1 to 38, the encapsulation device has a weld spacing that is less than 9 mm apart from each other.

According to another Aspect (Aspect 40”) further to any one of Aspects 1 to 39, at least a portion of the solid features of the second layer are in contact with the first layer are bonded solid features.

According to another Aspect (Aspect 41”) further to any one of Aspects 1 to 40, the second layer has a pore size from about 1 micron to about 9 microns in effective diameter.

According to another Aspect (“Aspect 42”) further to any one of Aspects 1 to 41, the encapsulation device has a surface coating thereon where the surface coating is one or more members selected from antimicrobial agents, antibodies, pharmaceuticals and biologically active molecules.

According to another Aspect (“Aspect 43”) further to any one of Aspects 1 to 41, the encapsulation device has a hydrophilic coating thereon.

In one Aspect (“Aspect 44”) an encapsulation device includes (1) at least one biocompatible membrane composite sealed along a portion of its periphery to define at least one lumen therein, the lumen having opposing surfaces and (2) at least one filling tube in fluid communication with the lumen, where the at least one biocompatible membrane composite includes a first layer and a second layer having a majority of solid features with a majority of solid feature spacing less than about 50 microns, and where a maximum oxygen diffusion distance is from about 25 microns to about 500 microns.

According to another Aspect (“Aspect 45”) further to Aspect 44, the first layer has a mass per area (MpA) less than about 5 g/m².

According to another Aspect (“Aspect 46”) further to Aspect 44 or Aspect 45, the first layer has an MPS (maximum pore size) less than about 1 micron.

According to another Aspect (“Aspect 47”) further to any one of Aspects 44 to 46, the at least one biocompatible membrane composite has a maximum tensile load in the weakest axis greater than 40 N/m.

According to another Aspect (“Aspect 48”) further to any one of Aspects 44 to 47, the first layer has a first porosity greater than about 50%.

According to another Aspect (“Aspect 49”) further to any one of Aspects 44 to 48, the second layer has a second porosity greater than about 60%.

According to another Aspect (“Aspect 50”) further to any one of Aspects 44 to 49, the second layer has a thickness less than about 200 microns.

According to another Aspect (“Aspect 51”) further to any one of Aspects 44 to 50, the solid features of the second layer each include a representative minor axis, a representative major axis, and a solid feature depth where a majority of at least two of the representative minor axis, the representative major axis, and the solid feature depth of the second layer is greater than about 5 microns.

According to another Aspect (“Aspect 52”) further to any one of Aspects 44 to 51, the second layer has a pore size from about 1 micron to about 9 microns in effective diameter.

According to another Aspect (“Aspect 53”) further to any one of Aspects 44 to 52, the solid features are connected by fibrils and the fibrils are deformable.

According to another Aspect (“Aspect 54”) further to any of Aspects 44 to 53, at least a portion of the first solid features in contact with the first layer are bonded solid features.

According to another Aspect (“Aspect 55”) further to Aspect 54, a majority of the bonded features has a representative minor axis from about 3 microns to about 20 microns.

According to another Aspect (“Aspect 56”) further to any one of Aspects 44 to 55, the first layer and the second layer are intimately bonded.

According to another Aspect (“Aspect 57”) further to any one of Aspects 44 to 56, at least one of the first layer and the second layer includes a polymer, a fluoropolymer membrane, a non-fluoropolymer membrane, a woven textile, a non-woven textile, a woven or non-woven collections of fibers or yarns, a fibrous matrix, a spunbound non-woven material, and combinations thereof.

According to another Aspect (“Aspect 58”) further to any one of Aspects 44 to 57, at least one of the first layer and the second layer is a polymer selected from an expanded polytetrafluoroethylene (ePTFE) membrane, a fluorinated ethylene propylene (FEP) membrane and a modified ePTFE membrane.

According to another Aspect (“Aspect 59”) further to any one of Aspects 44 to 58, at least one of the first layer and the second layer is an expanded polytetrafluoroethylene membrane.

According to another Aspect (“Aspect 60”) further to any one of Aspects 44 to 59, the second layer includes at least one of a textile and a non-fluoropolymer membrane.

According to another Aspect (“Aspect 61”) further to Aspect 60, the textile is selected from woven textiles, non-woven textiles, spunbound materials, melt blown fibrous materials, and electrospun nanofibers.

According to another Aspect (“Aspect 62”) further to Aspect 60, the non-fluoropolymer membrane is selected from polyvinylidene difluoride, nanofibers, polysulfones, polyethersulfones, polyarlysulfones, polyether ether ketone, polyethylenes, polypropylenes, polyimides and combinations thereof.

According to another Aspect (“Aspect 63”) further to any one of Aspects 44 to 62, the second layer includes expanded polytetrafluoroethylene.

According to another Aspect (“Aspect 64”) further to any one of Aspects 44 to 63, the second layer includes nodes, and the nodes are the solid features.

According to another Aspect (“Aspect 65”) further to any one of Aspects 44 to 64, including a reinforcing component.

According to another Aspect (“Aspect 66”) further to Aspect 65, the reinforcing component is an external reinforcing component on the second layer.

According to another Aspect (“Aspect 67”) further to Aspect 65 or Aspect 66, the external reinforcing component has a stiffness from about 0.01 N/cm to about 3 N/cm.

According to another Aspect (“Aspect 68”) further to any one of Aspects 65 to 67, the external reinforcing component includes a spunbound polyester non-woven material.

According to another Aspect (“Aspect 69”) further to any one of Aspects 65 to 68, the external reinforcing component is a polyester woven mesh.

According to another Aspect (“Aspect 70”) further to Aspect 65, including an internal reinforcing component.

According to another Aspect (“Aspect 71”) further to Aspect 70, the internal reinforcing component has a stiffness from about 0.05 N/cm to about 5 N/cm.

According to another Aspect (“Aspect 72”) further to Aspect 70 or Aspect 71, the internal reinforcing component is a cell and nutrient impermeable reinforcing component.

According to another Aspect (“Aspect 73”) further to any one of Aspects 70 to 72, the internal reinforcing component is substantially centrally located within the encapsulation device and divides the lumen substantially in half.

According to another Aspect (“Aspect 74”) further to any one of Aspects 70 to 73, the internal reinforcing component has thereon structural pillars.

According to another Aspect (“Aspect 75”) further to any one of Aspects 70 to 74, including point bonds between the internal reinforcing component and the at least one biocompatible membrane composite.

According to another Aspect (“Aspect 76”) further to any one of Aspects 44 to 75, wherein the encapsulation device includes (1) a first biocompatible membrane composite and a second biocompatible membrane composite and (2) point bonds between the first and second biocompatible membrane composites.

According to another Aspect (“Aspect 77”) further to any one of Aspects 44 to 76, including point bonds having a diameter from about 1 mm diameter and where the point bonds are spaced from about 0.5 mm to about 9 mm from each other.

According to another Aspect (“Aspect 78”) further to any one of Aspects 44 to 77, including a cell displacing core disposed in the lumen.

According to another Aspect (“Aspect 79”) further to any one of Aspects 44 to 78, including polymeric structural spacers interconnecting opposing layers of the lumen.

According to another Aspect (“Aspect 80”) further to any one of Aspects 44 to 79, the encapsulation device is formed with one or more of a lap seam, a butt seam or a fin seam.

According to another Aspect (“Aspect 81”) further to any one of Aspects 44 to 80, including structural spacers located within the lumen to maintain a desired thickness of the lumen.

According to another Aspect (“Aspect 82”) further to any one of Aspects 44 to 81, the encapsulation device has a weld spacing that is less than 9 mm from each other.

According to another Aspect (“Aspect 83”) further to any one of Aspects 44 to 82, the encapsulation device has a surface coating thereon, the surface coating being one or more members selected from antimicrobial agents, antibodies, pharmaceuticals, and biologically active molecules.

According to another Aspect (“Aspect 84”) further to any one of Aspects 44 to 83, the encapsulation device has a hydrophilic coating thereon.

In one Aspect (“Aspect 85) an encapsulation device includes (1) a first biocompatible membrane composite sealed along the perimeter thereof to a second biocompatible membrane composite to define at least one lumen having a first interior surface and a second interior surface with a weld spacing less than 9 mm from each other, (2) an external reinforcing component with a stiffness greater than about 0.01 N/cm, and (3) at least one filling tube in fluid communication with the lumen, where at least one of the first and second biocompatible membrane composite includes a first layer and a second layer having solid features with a majority of solid feature spacing less than about 50 microns, where the first interior surface is spaced from the second interior surface within the lumen.

According to another Aspect (“Aspect 86”) further to Aspect 85, the first layer has a mass per area (MpA) less than about 5 g/m².

According to another Aspect (“Aspect 87”) further to Aspect 85 or Aspect 860 the first layer has an MPS (maximum pore size) less than about 1 micron.

According to another Aspect (“Aspect 88) further to any one of Aspects 85 to 87, at least one of the first biocompatible membrane and the second biocompatible membrane composite has a maximum tensile load in the weakest axis greater than 40 N/m.

According to another Aspect (“Aspect 89”) further to any one of Aspects 85 to 88, the first layer has a first porosity greater than about 50%.

According to another Aspect (“Aspect 90”) further to any one of Aspects 85 to 89, the second layer has a second porosity greater than about 60%.

According to another Aspect (“Aspect 91”) further to any one of Aspects 85 to 90, the second layer has a thickness less than about 200 microns.

According to another Aspect (“Aspect 92”) further to any one of Aspects 85 to 91, the solid features of the second layer each include a representative minor axis, a representative major axis, and a solid feature depth where a majority of at least two of the second layer representative minor axis where the representative major axis, and the solid feature depth of the second layer is greater than about 5 microns.

According to another Aspect (“Aspect 93”) further to any one of Aspects 85 to 92, the second layer has a pore size from about 1 micron to about 9 microns in effective diameter.

According to another Aspect (“Aspect 94”) further to any one of Aspects 85 to 93, the solid features are connected by fibrils and the fibrils are deformable.

According to another Aspect (“Aspect 95”) further to any one of Aspects 85 to 94, at least a portion of the first solid features in contact with the first layer are bonded solid features.

According to another Aspect (“Aspect 96”) further to Aspect 95, a majority of the bonded features has a representative minor axis from about 3 microns to about 20 microns.

According to another Aspect (“Aspect 97”) further to any one of Aspects 85 to 96, the first layer and the second layer are intimately bonded.

According to another Aspect (“Aspect 98”) further to any one of Aspects 85 to 97, at least one of the first layer and the second layer includes a polymer, a fluoropolymer membrane, a non-fluoropolymer membrane, a woven textile, a non-woven textile, a woven or non-woven collections of fibers or yarns, a fibrous matrix, a spunbound non-woven material, and combinations thereof.

According to another Aspect (“Aspect 99”) further to any one of Aspects 85 to 98, at least one of the first layer and the second layer is a polymer selected from an expanded polytetrafluoroethylene (ePTFE) membrane, a fluorinated ethylene propylene (FEP) membrane and a modified ePTFE membrane.

According to another Aspect (“Aspect 100”) further to any one of Aspects 85 to 99, at least one of the first layer and the second layer is an expanded polytetrafluoroethylene membrane.

According to another Aspect (“Aspect 101”) further to any one of Aspects 85 to 100, the second layer includes at least one of a textile and a non-fluoropolymer membrane.

According to another Aspect (“Aspect 102”) further to Aspect 101, the textile is selected from woven textiles, non-woven textiles, spunbound materials, melt blown fibrous materials, and electrospun nanofibers.

According to another Aspect (“Aspect 103”) further to any one of Aspects 85 to 102 the non-fluoropolymer material is selected from polyvinylidene difluoride, nanofibers, polysulfones, polyethersulfones, polyarlysulfones, polyether ether ketone, polyethylenes, polypropylenes, polyimides and combinations thereof. According to another Aspect (“Aspect 104”) further to any one of Aspects 85 to 103, the second layer includes an expanded polytetrafluoroethylene membrane.

According to another Aspect (“Aspect 105”) further to any one of Aspects 85 to 1046, the solid features include nodes, and wherein the nodes are the solid features.

According to another Aspect (“Aspect 106”) further to any one of Aspects 87 to 105, including a reinforcing component.

According to another Aspect (“Aspect 107”) further to Aspect 106, the reinforcing component is an external reinforcing component.

According to another Aspect (“Aspect 108”) further to Aspect 106 or Aspect 107, the external reinforcing component has a stiffness from about 0.01 N/cm to about 3 N/cm.

According to another Aspect (“Aspect 109”) further to any one of Aspects 106 to 108, the external reinforcing component includes a spunbound polyester non-woven material.

According to another Aspect (“Aspect 110”) further to any one of Aspects 106 to 109, the external reinforcing component is a polyester woven mesh.

According to another Aspect (“Aspect 111”) further to Aspect 106, the reinforcing component is an internal reinforcing component.

According to another Aspect (“Aspect 112”) further to Aspect 111, the internal reinforcing component has a stiffness from 0.05 N/cm to about 5 N/cm.

According to another Aspect (“Aspect 113”) further to Aspect 111 or Aspect 112, the internal reinforcing component is a cell and nutrient impermeable reinforcing component.

According to another Aspect (“Aspect 114”) further to any one of Aspects 111 to 113, the internal reinforcing component is substantially centrally located within the encapsulation device and divides the lumen substantially in half.

According to another Aspect (“Aspect 115”) further to any one of Aspects 111 to 114, the internal reinforcing component has thereon structural pillars.

According to another Aspect (“Aspect 116”) further to any one of Aspects 111 to 115, including point bonds between the internal reinforcing component and at least one of the first and second biocompatible membrane composites.

According to another Aspect (“Aspect 117”) further to any one of Aspects 85 to 116, including point bonds between the first biocompatible membrane composite and second biocompatible membrane composite.

According to another Aspect (“Aspect 118”) further to any one of Aspects 85 to 117, the encapsulation device is formed with one or more of a lap seam, a butt seam or a fin seam.

According to another Aspect (“Aspect 119”) the encapsulation device of any one of claims 85 to 118, wherein the second layer of at least one of the first and second biocompatible membrane composites has therein solid features intimately bonded to a surface of the first layer.

According to another Aspect (“Aspect 120”) further to any one of Aspects 85 to 119, the encapsulation device has a surface coating thereon, where the surface coating is one or more members selected from antimicrobial agents, antibodies, pharmaceuticals and biologically active molecules.

According to another Aspect (“Aspect 121”) further to any one of Aspects 85 to 120, the encapsulation device has a hydrophilic coating thereon.

In one Aspect (“Aspect 122”) an encapsulation device includes (1) a first biocompatible membrane composite, (1) a second biocompatible membrane composite, (3) a reinforcing component having a stiffness from about 0.01 N/cm to about 5 N/cm, and (4) a perimeter seal, and (5) a weld spacing of the perimeter seal of less than 9 mm from each other, where a majority of at least one of the first and second biocompatible membrane composites include a first layer and a second layer having solid features with a majority of a solid feature spacing less than about 50 microns.

According to another Aspect (“Aspect 123”) further to Aspect 122, the first layer has a mass per area (MpA) less than about 5 g/m².

According to another Aspect (“Aspect 124”) further to Aspect 122 or Aspect 123, the first layer has an MPS (maximum pore size) less than about 1 micron.

According to another Aspect (“Aspect 125”) further to any one of Aspects 123 to 124, at least one of the first biocompatible membrane composite and the second biocompatible membrane composite has a maximum tensile load in the weakest axis greater than 40 N/m.

According to another Aspect (“Aspect 126) further to any one of Aspects 123 to 125 the first layer has a first porosity greater than about 50%. According to another Aspect (“Aspect 127”) further to any one of Aspects 123 to 126, the second layer has a second porosity greater than about 60%.

According to another Aspect (“Aspect 128”) further to any one of Aspects 123 to 127, the second layer has a thickness less than about 200 microns.

According to another Aspect (“Aspect 129”) further to any one of Aspects 123 to 128, the solid features of the second layer each include a representative minor axis, a representative major axis, and a solid feature depth where a majority of at least two of the representative minor axis, the representative major axis, and the solid feature depth of the second layer is greater than about 5 microns.

According to another Aspect (“Aspect 130”) further to any one of Aspects 123 to 129, the second layer has a pore size from about 1 micron to about 9 microns in effective diameter.

According to another Aspect (“Aspect 131”) further to any one of Aspects 123 to 130, the solid features are connected by fibrils and the fibrils are deformable.

According to another Aspect (“Aspect 132”) further any one of Aspects 123 to 131, at least a portion of the first solid features in contact with the first layer are bonded solid features.

According to another Aspect (“Aspect 133”) further Aspect 132, a majority of the bonded solid features has a representative minor axis from about 3 microns to about 20 microns.

According to another Aspect (“Aspect 134”) further to any one of Aspects 123 to 133, the first layer and the second layer are intimately bonded.

According to another Aspect (“Aspect 135”) further to any one of Aspects 123 to 134, at least one of the first layer and the second layer includes a polymer, a fluoropolymer membrane, a non-fluoropolymer membrane, a woven textile, a non-woven textile, a woven or non-woven collections of fibers or yarns, a fibrous matrix, a spunbound non-woven material, and combinations thereof.

According to another Aspect (“Aspect 136”) further to any one of Aspects 123 to 135, at least one of the first layer and the second layer is a polymer selected from an expanded polytetrafluoroethylene (ePTFE) membrane, a fluorinated ethylene propylene (FEP) membrane and a modified ePTFE membrane.

According to another Aspect (“Aspect 137”) further to any one of Aspects 123 to 136, at least one of the first layer and the second layer is an expanded polytetrafluoroethylene membrane.

According to another Aspect (“Aspect 138”) further to any one of Aspects 123 to 137, the second layer includes at least one of a textile and a non-fluoropolymer membrane.

According to another Aspect (“Aspect 139”) further to Aspect 138, the textile is selected from woven textiles, non-woven textiles, spunbound materials, melt blown fibrous materials, and electrospun nanofibers.

According to another Aspect (“Aspect 140”) further to Aspect 138, the non-fluoropolymer membrane is selected from polyvinylidene difluoride, nanofibers, polysulfones, polyethersulfones, polyarlysulfones, polyether ether ketone, polyethylenes, polypropylenes, polyimides, and combinations thereof.

According to another Aspect (“Aspect 141”) further to any one of Aspects 123 to 140, the second layer includes an expanded polytetrafluoroethylene membrane.

According to another Aspect (“Aspect 142”) further to any one of Aspects 123 to 141, the second layer includes nodes, and where the nodes are the solid features.

According to another Aspect (“Aspect 143”) further to any one of Aspects 123 to 142, the reinforcing component is a cell and nutrient impermeable reinforcing component.

According to another Aspect (“Aspect 144”) further to any one of Aspects 123 to 143, the reinforcing component is substantially centrally located within the encapsulation device and divides the lumen substantially in half.

According to another Aspect (“Aspect 145”) further to any one of Aspects 123 to 144, the reinforcing component has thereon structural pillars.

According to another Aspect (“Aspect 146”) further to any one of Aspects 123 to 145, including point bonds between the first biocompatible membrane composite and the second biocompatible membrane composite.

According to another Aspect (“Aspect 147”) further to Aspect 146, the point bonds have a diameter of about 1 mm and are spaced from about 0.5 mm to about 9 mm from each other.

According to another Aspect (“Aspect 148”) further to any one of Aspects 123 to 147 the encapsulation device is formed with one or more of a lap seam, a butt seam or a fin seam.

According to another Aspect (“Aspect 149”) further to any one of Aspects 123 to 152, the encapsulation device has a surface coating thereon, the surface coating being one or more members selected from antimicrobial agents, antibodies, pharmaceuticals and biologically active molecules.

According to another Aspect (“Aspect 150”) further to any one of Aspects 123 to 149, the encapsulation device has a hydrophilic coating thereon.

In one Aspect (“Aspect 151”) an encapsulation device includes (1) a biocompatible membrane composite sealed along first opposing edges to itself and sealed along its periphery on second opposing edges to form a lumen and (2) at least one fill tube in fluid communication with the lumen, where the biocompatible membrane composite includes a first layer, and a second layer having a majority of solid features with a majority of solid feature spacing less than about 50 microns.

According to another Aspect (“Aspect 152”) further to Aspect 151, the first layer has a mass per area (MpA) less than about 5 g/m².

According to another Aspect (“Aspect 153”) further to Aspect 151 or Aspect 152, the first layer has an MPS (maximum pore size) less than about 1 micron.

According to another Aspect (“Aspect 154”) further to any one of Aspects 151 to 153, the biocompatible membrane composite has a maximum tensile load in the weakest axis greater than 40 N/m.

According to another Aspect (“Aspect 155”) further to any one of Aspects 151 to 158154, the first layer has a first porosity greater than about 50%.

According to another Aspect (“Aspect 156”) further to any one of Aspects 151 to 155, the second layer has a second porosity greater than about 60%.

According to another Aspect (“Aspect 157”) further to any one of Aspects 151 to 156, the second layer has a thickness less than about 200 microns.

According to another Aspect (“Aspect 158”) further to any one of Aspects 151 to 157, the solid features of the second layer each include a representative minor axis, a representative major axis, and a solid feature depth where a majority of at least two of the second layer representative minor axis, the representative major axis, and the solid feature depth of the second layer is greater than about 5 microns.

According to another Aspect (“Aspect 159”) further to any one of Aspects 151 to 158, the second layer has a pore size from about 1 micron to about 9 microns in effective diameter.

According to another Aspect (“Aspect 160”) further to any one of Aspects 151 to 159, the solid features are connected by fibrils and the fibrils are deformable.

According to another Aspect (“Aspect 161”) further to any one of Aspects 151 to 160, at least a portion of the first solid features in contact with the first layer are bonded solid features.

According to another Aspect (“Aspect 162”) further to Aspect 161, a majority of the bonded features has a representative minor axis from about 3 microns to about 20 microns.

According to another Aspect (“Aspect 163”) further to any one of Aspects 151 to 162, the first layer and the second layer are intimately bonded.

According to another Aspect (“Aspect 164”) the encapsulation device of any one of Aspects 151 to 163, at least one of the first layer and the second layer comprises a polymer, a fluoropolymer membrane, a non-fluoropolymer membrane, a woven textile, a non-woven textile, a woven or non-woven collections of fibers or yarns, a fibrous matrix, a spunbound non-woven material, and combinations thereof.

According to another Aspect (“Aspect 165”) the encapsulation device of any one of Aspects 151 to 164, at least one of the first layer and the second layer is a polymer selected from an expanded polytetrafluoroethylene (ePTFE) membrane, a fluorinated ethylene propylene (FEP) membrane and a modified ePTFE membrane.

According to another Aspect (“Aspect 166”) further to one of Aspects 151 to 165, at least one of the first layer and the second layer is an expanded polytetrafluoroethylene membrane.

According to another Aspect (“Aspect 167”) further to one of Aspects 151 to 166 the second layer includes at least one of a textile and a non-fluoropolymer membrane.

According to another Aspect (“Aspect 168”) further to Aspect 167, the textile is selected from woven textiles, non-woven textiles, spunbound materials, melt blown fibrous materials, and electrospun nanofibers.

According to another Aspect (“Aspect 169”) further to Aspect 167, the non-fluoropolymer membrane is selected from polyvinylidene difluoride, nanofibers, polysulfones, polyethersulfones, polyarlysulfones, polyether ether ketone, polyethylenes, polypropylenes, polyimides, and combinations thereof.

According to another Aspect (“Aspect 170”) further to any one of Aspects 151 to 167, at least one of the first layer and the second layer includes a polymer, a fluoropolymer membrane, a non-fluoropolymer membrane, a woven textile, a non-woven textile, a woven or non-woven collections of fibers or yarns, a fibrous matrix, a spunbound non-woven material, and combinations thereof.

According to another Aspect (“Aspect 171”) further to any one of Aspects 151 to 170, at least one of the first layer and the second layer is a polymer selected from an expanded polytetrafluoroethylene (ePTFE) membrane, a fluorinated ethylene propylene (FEP) membrane and a modified ePTFE membrane.

According to another Aspect (“Aspect 172”) further to any one of Aspects 151 to 171, the second layer includes expanded polytetrafluoroethylene.

According to another Aspect (“Aspect 173”) further to any one of Aspects 151 to 172, the second layer includes nodes, and where the nodes are the solid features.

According to another Aspect (“Aspect 174”) further to any one of Aspects 151 to 174, including an internal reinforcing component.

According to another Aspect (“Aspect 175”) further to Aspect 174, the internal reinforcing component has a stiffness from about 0.05 N/cm to about 5 N/cm.

According to another Aspect (“Aspect 176”) further to Aspect 174 or Aspect 175, the internal reinforcing component is a cell and nutrient impermeable reinforcing component.

According to another Aspect (“Aspect 177”) further to any one of Aspects 174 to 176, the internal reinforcing component is a cell displacing core disposed in the lumen.

According to another Aspect (“Aspect 178”) further to any one of Aspects 151 to 177 the encapsulation device is formed with one or more of a lap seam, a butt seam or a fin seam.

According to another Aspect (“Aspect 179”) further to any one of Aspects 151 to 178, the encapsulation device has a weld spacing that is less than 9 mm from each other.

According to another Aspect (“Aspect 180”) further to any one of Aspects 151 to 179, the encapsulation device has a surface coating thereon, the surface coating being one or more members selected from antimicrobial agents, antibodies, pharmaceuticals and biologically active molecules.

According to another Aspect (“Aspect 181”) further to any one of Aspects 151 to 180, the encapsulation device has a hydrophilic coating thereon.

According to another Aspect (“Aspect 182”) further to any one of the preceding Aspects, a method for lowering blood glucose levels in a mammal includes transplanting a cell encapsulated device including a biocompatible membrane composite of any of the previous claims, wherein cells encapsulated therein include a population of PDX1-positive pancreatic endoderm cells, and wherein the pancreatic endoderm cells mature into insulin secreting cells, thereby lowering blood glucose.

According to another Aspect (“Aspect 183”) further to any one of the preceding Aspects, the PDX1-positive pancreatic endoderm cells include a mixture of cells further including endocrine and/or endocrine precursor cells, wherein the endocrine and/or endocrine precursor cells express chromogranin A (CHGA).

According to another Aspect (“Aspect 184”) further to any one of the preceding Aspects, a method for lowering blood glucose levels in a mammal includes transplanting a cell encapsulation device as in claim 1, wherein cells encapsulated therein include a population of PDX1-positive pancreatic endoderm cells, and wherein the pancreatic endoderm cells mature into insulin secreting cells, thereby lowering blood glucose.

According to another Aspect (“Aspect 185”) further to any one of the preceding Aspects, the PDX1-positive pancreatic endoderm cells include a mixture of cells further including endocrine and/or endocrine precursor cells, wherein the endocrine and/or endocrine precursor cells express chromogranin A (CHGA).

According to another Aspect (“Aspect 186”) further to any one of the preceding Aspects, a method for lowering blood glucose levels in a mammal includes transplanting a cell encapsulation device including a biocompatible membrane composite that includes a first layer and a second layer having solid features with a solid feature spacing less than about 50 microns, and a cell population including PDX1-positive pancreatic endoderm cells, and wherein the pancreatic endoderm cells mature into insulin secreting cells, thereby lowering blood glucose, where the encapsulation device has a majority oxygen diffusion distance of less than 300 microns, and in particular less than about 150 microns.

According to another Aspect (“Aspect 187”) further to any one of the preceding Aspects, the PDX1-positive pancreatic endoderm cells include a mixture of cells further including endocrine and/or endocrine precursor cells, wherein the endocrine and/or endocrine precursor cells express chromogranin A (CHGA).

According to another Aspect (“Aspect 188”) further to any one of the preceding Aspects, a method for lowering blood glucose levels in a mammal includes transplanting a biocompatible membrane composite that includes a first layer, a second layer having solid features with a solid feature spacing less than about 50 microns, and a cell population including PDX1-positive pancreatic endoderm cells, and wherein the pancreatic endoderm cells mature into insulin secreting cells, thereby lowering blood glucose, wherein the encapsulation device has a majority oxygen diffusion distance of less than 300 microns.

According to another Aspect (“Aspect 189”) further to any one of the preceding Aspects, the PDX1-positive pancreatic endoderm cells include a mixture of cells further including endocrine and/or endocrine precursor cells, wherein the endocrine and/or endocrine precursor cells express chromogranin A (CHGA).

According to another Aspect (“Aspect 190”) further to any one of the preceding Aspects, an encapsulated in vitro PDX1-positive pancreatic endoderm cells include a mixture of cell sub-populations including at least a pancreatic progenitor population co-expressing PDX-1/NKX6.1.

According to another Aspect (“Aspect 191”) further to any one of the preceding Aspects, an encapsulated in vitro PDX1-positive pancreatic endoderm cells includes a mixture of cell sub-populations including at least a pancreatic progenitor population co-expressing PDX-1/NKX6.1 and a pancreatic endocrine and/or endocrine precursor population expressing PDX-1/NKX6.1 and CHGA.

According to another Aspect (“Aspect 192”) further to any one of the preceding Aspects, at least 30% of the population includes pancreatic progenitor population co-expressing PDX-1/NKX6.1.

According to another Aspect (“Aspect 193”) further to any one of the preceding Aspects, at least 40% of the population includes pancreatic progenitor population co-expressing PDX-1/NKX6.1.

According to another Aspect (“Aspect 194”) further to any one of the preceding Aspects, at least 50% of the population includes pancreatic progenitor population co-expressing PDX-1/NKX6.1.

According to another Aspect (“Aspect 195”) further to any one of the preceding Aspects, at least 20% of the population endocrine and/or endocrine precursor population express PDX-1/NKX6.1/CHGA.

According to another Aspect (“Aspect 196”) further to any one of the preceding Aspects, at least 30% of the population endocrine and/or endocrine precursor population express PDX-1/NKX6.1/CHGA.

According to another Aspect (“Aspect 197”) further to any one of the preceding Aspects, at least 40% of the population endocrine and/or endocrine precursor population express PDX-1/NKX6.1/CHGA.

According to another Aspect (“Aspect 198”) further to any one of the preceding Aspects, the pancreatic progenitor cells and/or endocrine or endocrine precursor cells are capable of maturing into insulin secreting cells in vivo.

According to another Aspect (“Aspect 199”) further to any one of the preceding Aspects, a method for producing insulin in vivo includes transplanting a cell encapsulated device including a biocompatible membrane composite of any one of the previous claims and a population of PDX-1 pancreatic endoderm cells mature into insulin secreting cells, wherein the insulin secreting cells secrete insulin in response to glucose stimulation.

According to another Aspect (“Aspect 200”) further to any one of the preceding Aspects, the PDX1-positive pancreatic endoderm cells include a mixture of cells further including endocrine and/or endocrine precursor cells, wherein the endocrine and/or endocrine precursor cells express chromogranin A (CHGA).

According to another Aspect (“Aspect 201”) further to any one of the preceding Aspects, at least about 30% of the population are endocrine and/or endocrine precursor population expressing PDX-1/NKX6.1/CHGA.

According to another Aspect (“Aspect 202”) further to any one of the preceding Aspects, an in vitro human PDX1-positive pancreatic endoderm cell culture includes a mixture of PDX-1 positive pancreatic endoderm cells and at least a transforming growth factor beta (TGF-beta) receptor kinase inhibitor.

According to another Aspect (“Aspect 203”) further to any one of the preceding Aspects, further including a bone morphogenetic protein (BMP) inhibitor.

According to another Aspect (“Aspect 204”) further to any one of the preceding Aspects, the TGF-beta receptor kinase inhibitor is TGF-beta receptor type 1 kinase inhibitor.

According to another Aspect (“Aspect 205”) further to any one of the preceding Aspects, the TGF-beta receptor kinase inhibitor is ALK5i.

According to another Aspect (“Aspect 206”) further to any one of the preceding Aspects, the BMP inhibitor is noggin.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1A is a schematic illustration depicting the determination of solid feature spacing where three neighboring solid features represent the corners of a triangle whose circumcircle has an interior devoid of additional solid features and the solid feature spacing is the straight distance between two of the solid features forming the triangle in accordance with embodiments described herein;

FIG. 1B is a schematic illustration depicting the determination of non-neighboring solid features where the solid features form the corners of a triangle whose circumcircle contains at least one additional solid feature in accordance with embodiments described herein;

FIG. 2 is a scanning electron micrograph of the spacing (white lines) between solid features (white shapes) in an expanded polytetrafluoroethylene (ePTFE) membrane in accordance with embodiments described herein;

FIG. 3A is a schematic illustration depicting the method to determine the major axis and the minor axis of a solid feature in accordance with embodiments described herein;

FIG. 3B is a schematic illustration depicting the depth of a solid feature in accordance with embodiments described herein;

FIG. 4 is a schematic illustration of the effective diameter of a pore in accordance with embodiments described herein;

FIG. 5 is a scanning electron micrograph (SEM) showing pore size according to embodiments described herein;

FIG. 6A is a schematic illustration of a thermoplastic polymer in the form of solid features positioned on the surface of a cell impermeable layer in accordance with embodiments described herein;

FIGS. 6B-6I are schematic illustrations of sample geometries for forming solid features on a cell impermeable layer in accordance with embodiments described herein;

FIG. 7A is a schematic illustration of a biocompatible membrane composite having therein bonded solid features intimately bonded to the surface of the cell impermeable layer in accordance with embodiments described herein;

FIG. 7B is a schematic illustration of a biocompatible membrane composite where the mitigation layer has therein solid features with differing heights and widths in accordance with embodiments described herein;

FIG. 8 is a schematic illustration of a biocompatible membrane composite having a mitigation layer containing therein solid features that are nodes in accordance with embodiments described herein;

FIGS. 9A-9C are schematic illustrations of a biocompatible membrane composites showing various locations of a reinforcing component in accordance with embodiments described herein;

FIG. 10 is a schematic illustration of a cross-sectional view of a mitigation layer positioned on a cell impermeable layer where the mitigation layer is characterized at least by solid feature size, solid feature spacing, solid feature depth, and thickness in accordance with embodiments described herein;

FIG. 11 is a schematic illustration of a cross-sectional view of a mitigation layer positioned on a cell impermeable layer where the mitigation layer is characterized at least by solid feature size, solid feature spacing, solid feature depth, thickness, and pore size in accordance with embodiments described herein;

FIG. 12A is a schematic illustration of a top view of a cell encapsulation device in accordance with embodiments described herein;

FIG. 12B is a schematic illustration of the cross-section of a cell encapsulation device showing the lumen and the oxygen diffusion distance (ODD) in accordance with embodiments described herein;

FIG. 13 is a schematic illustration depicting an exploded view of an encapsulation device according to embodiments described herein;

FIG. 14 is a scanning electron micrograph (SEM) image of the top surface of a comparable cell impermeable layer formed of an expanded polytetrafluoroethylene (ePTFE) membrane in accordance with embodiments described herein;

FIG. 15 is an SEM image of the top surface of the ePTFE mitigation layer with a discontinuous layer of fluorinated ethylene propylene (FEP) thereon in Example 1 in accordance with embodiments described herein;

FIG. 16 is an SEM image of the top surface of the ePTFE cell impermeable layer used in Example 1 in accordance with embodiments described herein;

FIG. 17 is an SEM image of the top surface of the ePTFE mitigation layer in Example 1 in accordance with embodiments described herein;

FIG. 18 is an SEM image of the cross-section of the two-layer ePTFE composite formed in Example 1 in accordance with embodiments described herein;

FIG. 19 is an SEM image of the top surface of the ePTFE cell impermeable layer used in Example 1 in accordance with embodiments described herein;

FIG. 20 is an SEM image of the top surface of the ePTFE mitigation layer used in Example 1 in accordance with embodiments described herein;

FIG. 21 is an SEM image of the cross-section of a two-layer ePTFE composite formed in Example 1 in accordance with embodiments described herein;

FIG. 22 is an SEM image of the top surface of a vascularization layer formed of a non-woven polyester in accordance with embodiments described herein;

FIG. 23A is a schematic illustration of Device A of Example 2 having a lumen width of 9.0 mm in accordance with embodiments described herein;

FIG. 23B is a schematic illustration of Device B of Example 2 having a lumen width of 7.2 mm in accordance with embodiments described herein;

FIG. 23C is a schematic illustration of Device C of Example 2 having a lumen width of 5.4 mm in accordance with embodiments described herein;

FIG. 24A is a representative histology image depicting a maximum graft thickness across the cross-section of Device A of Example 2 at 20 weeks in accordance with embodiments described herein;

FIG. 24B is a representative histology image depicting a maximum graft thickness across the cross-section of Device B of Example 2 at 20 weeks in accordance with embodiments described herein;

FIG. 25C is a representative histology image depicting a maximum graft thickness across the cross-section of Device C of Example 2 at 20 weeks in accordance with embodiments described herein;

FIG. 25 is an SEM image of the top surface of the ePTFE mitigation layer with a discontinuous layer of FEP thereon in Example 3 in accordance with embodiments described herein;

FIG. 26 is an SEM image of the top surface of the ePTFE vascularization layer utilized in Example 3 in accordance with embodiments described herein;

FIG. 27 is an SEM image of the cross-section of the three layer composite formed in Example 3 in accordance with embodiments described herein

FIG. 28 is a schematic illustration depicting an exploded view of a planar device in accordance with embodiments described herein;

FIG. 29 is a schematic illustration of a top view of a planar device in accordance with embodiments described herein;

FIG. 30A is an image of a top view of a surface of a planar device in accordance with embodiments described herein;

FIG. 30B is a representative histology image of the cross-section of the planar device of Example 3 depicting in vivo cell viability;

FIG. 31 is an image of a cross-section of the planar device of FIG. 30 taken along line A-A showing a single point bond and the lumen in accordance with embodiments described herein;

FIG. 32 is an image of a cross-section of the planar device of FIG. 30 taken along line B-B showing two point bonds and the lumen in accordance with embodiments described herein;

FIG. 33 is an SEM image of the top surface of the ePTFE vascularization layer with a discontinuous layer of FEP thereon in Example 4;

FIG. 34 is a representative SEM image of the node and fibril microstructure of one layer (Cell Impermeable Layer) of the ePTFE two-layer composite membrane of Example 4 in accordance with embodiments described herein;

FIG. 35 is representative SEM image of the node and fibril microstructure of the second ePTFE membrane (Mitigation Layer) of Example 4; in accordance with embodiments described herein;

FIG. 36 is a representative SEM image of the cross-section of the three layer biocompatible membrane composite utilized in Example 4 in accordance with embodiments described herein;

FIG. 37A is a top view of a reinforcing component with pillars in accordance with embodiments described herein;

FIG. 37B is a cross-section taken along A-A of FIG. 37A depicting a planar device with 250 microns pillars in accordance with embodiments described herein;

FIG. 37C is a cross-section taken along A-A of FIG. 37A depicting a planar device with 150 microns pillars in accordance with embodiments described herein;

FIG. 37D is a cross-section taken along A-A of FIG. 37A depicting a planar device with 75 microns pillars in accordance with embodiments described herein;

FIG. 37E is a representative histology image of the cross-section of Device A of Example 3 depicting an oxygen diffusion distance with in vivo cell viability in accordance with embodiments described herein;

FIG. 37F is a representative histology image of the cross-section of Device B of Example 3 depicting an oxygen diffusion distance with in vivo cell viability in accordance with embodiments described herein;

FIG. 38 is a schematic illustration of the geometry of a representative cell displacing core in accordance with embodiments described herein;

FIG. 39 is schematic illustration of a stainless steel mold in the shape of a final device in accordance with embodiments described herein;

FIG. 40A is an image of a tubular cell encapsulation device in accordance with embodiments described herein;

FIG. 40B is a schematic illustration of an exploded view of the tubular cell encapsulation device shown in FIG. 40A in accordance with embodiments described herein;

FIG. 41 is a schematic illustration of a portion of a planar cell encapsulation device in cross-section in accordance with embodiments described herein;

FIG. 42 is a schematic illustration of a portion of a cell encapsulation device having structural spacers positioned within the lumen in accordance with embodiments described herein in accordance with embodiments described herein;

FIG. 43 is a schematic illustration of a cell encapsulation device having a tubular shape and a tensioning member disposed within the lumen in accordance with embodiments described herein;

FIG. 44 is a schematic illustration of a cell encapsulating device that includes a tensioning member disposed within the lumen which contacts at least two opposing portions of the cell encapsulating device in accordance with embodiments described herein;

FIG. 45 is a schematic illustration of a cell encapsulating device that includes weld spacers in accordance with embodiments described herein;

FIG. 46 is schematic illustration of a cell encapsulating device that includes a tensioning member and a cell displacing core in accordance with embodiments described herein;

FIG. 47A is a schematic illustration of a lap seam in accordance with embodiments described herein;

FIG. 47B is a schematic illustration of a butt seam in accordance with embodiments described herein;

FIG. 47C is a schematic illustration of a fin seam in accordance with embodiments described herein;

FIG. 48 is a schematic illustration depicting the weld spacing (W) between the welded perimeters of a lumen of a cell encapsulation device in accordance with embodiments described herein;

FIG. 49A is a schematic illustration of the cross-section of the front view of a cell encapsulation device that includes a cell displacing core where the oxygen diffusion distance (ODD) is sufficiently narrow to provide conditions suitable for the survival and function of contained cells in accordance with embodiments described herein;

FIG. 49B is a schematic illustration of the cross-section of the side view of the cell encapsulation device of FIG. 49A in accordance with embodiments described herein;

FIG. 50 is a schematic illustration of a perspective view of the cell encapsulation device depicted in FIGS. 49A and 49B in accordance with embodiments described herein;

FIG. 51 is a schematic illustration of an encapsulation device that includes a plurality of interconnected encapsulation devices that are substantially parallel to each other along a length of the encapsulation device in accordance with embodiments described herein;

FIG. 52 is representative SEM image of the node and fibril microstructure of the external reinforcing component of Example 1 in accordance with embodiments described herein;

FIG. 53 is a representative histology image of Device A of Example 1 illustrating the presence of foreign body giant cells at the cell impermeable layer in accordance with embodiments described herein;

FIG. 54 is a representative histology image of Device B of Example 1 illustrating the absence of foreign body giant cells at the cell impermeable layer in accordance with embodiments described herein;

FIG. 55 is a representative histology image of a cross-section of a first cell encapsulation device of Example 5 depicting with in vivo cell viability in accordance with embodiments described herein;

FIG. 56 is a representative histology image of a cross-section of a first cell encapsulation device of Example 5 depicting with in vivo cell viability in accordance with embodiments described herein;

FIG. 57 is an image of the nitinol clip of Device 8B of Example 8;

FIG. 58 is an image of the reverse side of the nitinol clip of Device 8B of Example 8 in accordance with embodiments described herein;

FIG. 59 is an image of the nitinol sleeve of Device 8C of Example 8 in accordance with embodiments described herein;

FIG. 60 is a representative SEM image of the second ePTFE layer of Constructs A, B, and C of Example 8 having thereon FEP in accordance with embodiments described herein;

FIG. 61 is a representative SEM image of the node and fibril structure of the third ePTFE membrane in Construct A of Example 8 in accordance with embodiments described herein;

FIG. 62 is a representative SEM image of the node and fibril structure of the third ePTFE membrane in Construct B of Example 8 in accordance with embodiments described herein;

FIG. 63 is a representative SEM image of the node and fibril structure of the third ePTFE membrane in Construct C of Example 8 in accordance with embodiments described herein;

FIG. 64 is an SEM image of the cross-section of the biocompatible membrane composite of Construct A of Example 8 in accordance with embodiments described herein;

FIG. 65 is an SEM image of the cross-section of the biocompatible membrane composite of Construct B of Example 8 in accordance with embodiments described herein; and

FIG. 66 is an SEM image of the cross-section of the biocompatible membrane composite of Construct C of Example 8 in accordance with embodiments described herein.

### DETAILED DESCRIPTION

Persons skilled in the art will readily appreciate that various aspects of the present disclosure can be realized by any number of methods and apparatus configured to perform the intended functions. It should also be noted that the accompanying figures referred to herein are not necessarily drawn to scale, and may be exaggerated to illustrate various aspects of the present disclosure, and in that regard, the figures should not be construed as limiting. Directional references such as “up,” “down,” “top,” “left,” “right,” “front,” and “back,” among others are intended to refer to the orientation as illustrated and described in the figure (or figures) to which the components and directions are referencing. It is to be appreciated that the terms “biocompatible membrane composite” and “membrane composite” are used interchangeably herein. Additionally, the terms “cell encapsulation device”, “encapsulation device”, and “device” may be interchangeably used herein. It is to be noted that all ranges described herein are exemplary in nature and include any and all values in between. In addition, all references cited herein are incorporated by reference in their entireties.

The present disclosure is directed to cell encapsulation devices for biological entities and/or cell populations that contain at least one biocompatible membrane composite. The cell encapsulation devices are able to mitigate or tailor the foreign body response from the host such that sufficient blood vessels are able to form at a cell impermeable surface. Additionally, the encapsulation devices have an oxygen diffusion distance that is sufficient for the survival of the encapsulated cells so that the cells are able to secrete a therapeutically useful substance.

The biocompatible membrane composite includes a first layer and a second layer. Each layer is distinct and serves a necessary function for the survival of encapsulated cells. In certain embodiments, the first layer functions as a cell impermeable layer and the second layer functions as a mitigation layer. In some embodiment, the mitigation layer also acts as a vascularization layer. Herein, the term “first layer” is used interchangeably with “cell impermeable layer” and the term “second layer” is used interchangeably with “mitigation layer” for ease of convenience. The mitigation layer reduces the formation of foreign body giant cells on the surface of the cell impermeable layer. Other layers such as a vascularization layer, a mesh layer, a fabric layer, a reinforcing component on or within the biocompatible membrane composite may also be included as part of the cell encapsulation device. Herein, a “reinforcing component” may be further described as being external or internal and may be nutrient permeable or impermeable. For example, a reinforcing component may optionally be positioned on either side of the biocompatible membrane composite (i.e., external to) or within the biocompatible membrane composite (i.e., internal to) to provide support to and prevent distortion of the encapsulation device. It is to be appreciated that the term “about” as used herein denotes +/−10% of the designated unit of measure.

Biological entities suitable for use with the biocompatible membrane composite and the cell encapsulation devices made therewith, include, but are not limited to, cells, viruses, viral vectors, gene therapies, bacteria, proteins, polysaccharides, antibodies, and other bioactive entities. It is to be appreciated that if a biological entity other than a cell is selected for use herein, the bioactive component or product of the biological entity needs to be able to pass through the cell impermeable layer, but not the entity itself. For simplicity, herein the biological entity is referred to as a cell, but nothing in this description limits the biological entity to cells or to any particular type of cell, and the following description applies also to biological entities that are not cells.

Various types of prokaryotic cells, eukaryotic cells, mammalian cells, non-mammalian cells, and/or stem cells may be used with the biocompatible membrane composite described herein. In some embodiments, the cells secrete a therapeutically useful substance. Such therapeutically useful substances include hormones, growth factors, trophic factors, neurotransmitters, lymphokines, antibodies, or other cell products which provide a therapeutic benefit to the device recipient. Examples of such therapeutic cell products include, but are not limited to, hormones, growth factors, trophic factors, neurotransmitters, lymphokines, antibodies or other cell products which provide a therapeutic benefit to the device recipient. Examples of such therapeutic cell products include, but are not limited to, insulin and other pancreatic hormones, growth factors, interleukins, parathyroid hormone, erythropoietin, transferrin, collagen, elastin, tropoelastin, exosomes, vesicles, genetic fragments, and Factor VIII. Non-limiting examples of suitable growth factors include vascular endothelial growth factor, platelet-derived growth factor, platelet-activating factor, transforming growth factors bone morphogenetic protein, activin, inhibin, fibroblast growth factors, granulocyte-colony stimulating factor, granulocyte-macrophage colony stimulating factor, glial cell line-derived neurotrophic factor, growth differentiation factor-9, epidermal growth factor, and combinations thereof.

As discussed above, the biocompatible membrane composite includes a first layer (i.e., cell impermeable layer). The cell impermeable layer serves as a microporous, immune isolation barrier, and is impervious to vascular ingrowth and prevents cellular contact from the host. Herein, layers that restrict or prevent vascular ingrowth may be referred to as “tight” layers. Herein, layers that do not have openings large enough to allow cellular ingrowth may be referred to as “tight” layers. The pores of the cell impermeable layer are sufficiently small so as to allow the passage therethrough of cellular nutrients, oxygen, waste products, and therapeutic substances while not permitting the passage of any cells. In some embodiments, the cell impermeable layer has a maximum pore size (hereinafter MPS) that is less than about 1 micron, less than about 0.50 microns, less than about 0.30 microns, or less than about 0.10 microns as measured by porometry. The MPS may be from about 0.05 microns to about 1 micron, from about 0.1 microns to about 0.80 microns, from about 0.1 microns to about 0.6 microns, from about 0.1 microns to about 0.5 microns, or from about 0.2 microns to about 0.5 microns as measured by porometry.

Because the cell impermeable layer has an MPS that is sufficiently small so as to prevent vascular ingrowth, it is necessary to balance the parameters of the cell impermeable layer that could also negatively impact the mass transport and diffusion properties of the cell impermeable layer. For instance, while the MPS is small enough to prevent cell ingress or vascular ingrowth, the cell impermeable layer is sufficiently open so as to allow the passage of molecules (i.e. nutrients and therapeutic molecules) therethrough. Diffusion resistance is further minimized by keeping the cell impermeable layer thin and porous and low in mass. It is to be appreciated that sufficient durability and strength of the cell impermeable layer be maintained so that immune isolation can be provided in vivo through an intended use by ensuring the integrity of this tight layer. Therefore, it is necessary to balance the tradeoffs of the competing properties of strength and diffusion resistance.

In some embodiments, the cell impermeable layer has a thickness that is less than about 10 microns, less than about 8 microns, less than about 6 microns, or less than about 4 microns. The thickness of the cell impermeable layer may range from about 1 micron to about 10 microns, from about 1 micron to about 8 microns, from about 1 micron to about 6 microns, from about 5 microns to about 10 microns, or from about 1 micron to about 5 microns. In addition, it is to be appreciated that sufficient porosity of the cell impermeable layer be maintained so as to allow the passage of molecules. In certain embodiments, the porosity of the cell impermeable membrane is greater than about 50%, greater than about 60%, greater than about 70%, or greater than about 80%. Additionally, the porosity may range from about 50% to about 98%, from about 50% to about 90%, from about 50% to about 80%, or from about 60% to about 90%.

It is to be appreciated that sufficient durability and strength of the cell impermeable layer be maintained so that immune isolation can be provided in vivo through an intended use by ensuring the integrity of this tight layer. As the properties impacting diffusion resistance are minimized, it creates a trade-off in maintaining the necessary strength properties for integrity of the cell impermeable layer. In certain embodiments, the maximum tensile load of the weakest axis of the cell impermeable membrane is greater than about 40 N/m, greater than about 130 N/m, greater than about 260 N/m, greater than about 600 N/m, or greater than about 1000 N/m. Additionally, the maximum tensile load of the weakest axis may range from about 40 N/m to about 2000 N/m, from about 40 N/m to about 780 N/m, from about 40 N/m to about 350 N/m, from about 130 N/m to about 2000 N/m, from about 130 N/m to about 450 N/m, or from about 260 N/m to about 2000 N/m.

In certain embodiments, the cell impermeable membrane has a combination of tensile strengths in orthogonal directions (D1, D2) that result in a geometric mean tensile strength that is greater than about 20 MPa, greater than about 50 MPa, greater than about 100 MPa, or greater than about 150 MPa when the geometric mean tensile strength is defined per the following equation:

Geometric Mean=\sqrt{(Tensile Strength_{D1})²+(Tensile Strength_{D2})²}.

Additionally the geometric mean tensile strength may range from about 20 MPa to about 180 MPa, from about 30 MPa to about 150 MPa, from about 50 MPa to about 150 MPa, or from about 100 MPa to about 150 MPa.

The high intrinsic strength of the cell impermeable layer allows the cell impermeable layer to achieve the bulk strength necessary to remain retentive and robust in application while minimizing its thickness at porosities sufficient for nutrient transport. This enables cell impermeable layers with previously unobtainable combinations of thickness, porosity, and bulk strength, thereby enabling robust constructs with higher diffusion rates through reduced thickness.

As discussed previously, the biocompatible membrane composite contains a second layer (i.e., a mitigation layer). The mitigation layer is sufficiently porous to permit growth of vascular tissue into the mitigation layer, and therefore also acts as a vascularizing layer. The mitigation layer creates a suitable environment to minimize, reduce, inhibit, or even prevent the formation of foreign body giant cells while allowing for access to blood vessels directly at the cell impermeable layer. Ingrowth of vascular tissues into the mitigation layer facilitates nutrient transfer through the cell impermeable layer. Herein, layers that have openings large enough to allow vascular ingrowth may be referred to as “open” layers. Blood vessels, which are the source of oxygen and nutrients for implanted cells, need to form in the mitigation layer so that they are sufficiently close to the cell impermeable layer such that the distance for nutrient diffusion to any encapsulated cells is minimized. The thinness of the cell impermeable layer helps to reduce the distance over which diffusion must occur.

The ingrowth of vascular tissue through the mitigation layer up to the cell impermeable layer facilitates nutrient transfer across the cell impermeable layer. The mitigation layer creates an environment that enables a sufficient formation of blood vessels into the mitigation layer positioned adjacent to the cell impermeable layer instead of the formation of foreign body giant cells. As a result, and as shown in the Examples, foreign body giant cells do not form at the interface of the cell impermeable layer and the mitigation layer such that foreign body cells impede sufficient vascularization for cell survival. It is to be noted that foreign body giant cells may individually form at the interface of the cell impermeable layer and the mitigation layer, but they do not impede or prevent the vascularization needed for growth of encapsulated cells.

The mitigation layer is characterized at least in part by the inclusion of a plurality of solid features that have a solid feature spacing, which is discussed in detail below. “Solid features” as used herein may be defined as three dimensional components within the mitigation layer that are generally immovable and resistant to deformation when exposed to environmental forces, such as, but not limited to, cell movement (e.g., cellular migration and ingrowth, host vascularization/endothelial blood vessel formation). To facilitate the reduction or mitigation of the formation of a barrier of foreign body giant cells at the cell impermeable layer, the solid features abutting the surface of the cell impermeable layer adjacent to the mitigation layer help prevent the fusion of multiple macrophages into multinucleated foreign body giant cells at this interface. In some embodiments, the solid features in the mitigation layer abutting the cell impermeable layer are intimately bonded to the cell impermeable layer and are herein referred to as “bonded solid features”. “Non-bonded solid features” are those solid features within the mitigation layer that are not bonded (intimately bonded or otherwise) to the cell impermeable layer. “Intimate bond” and “intimately bonded” refer to layers of the biocompatible membrane composite or to solid features within the biocompatible membrane composite that are not readily separable or detachable at any point on their surface.

In some embodiments, the solid features of the mitigation layer project outwardly from a plane defined by the cell impermeable layer. In such embodiments, the solid features of the mitigation layer may be intimately bonded with the cell impermeable layer and spaced such that they provide blockades or barriers to the formation of foreign body giant cells at this tight, cell impermeable interface. In some embodiments the solid features may be a feature of the mitigation layer (e.g. nodes), and may be connected to each other, such as by fibrils or fibers. In another embodiments, the solid features may be provided and/or otherwise formed on the surface of the cell impermeable layer (e.g., printed solid features) such that the solid features project outwardly from a plane defined by plane defined by the cell impermeable layer.

In embodiments where the mitigation layer has a node and fibril microstructure (e.g., formed from a fibrillated polymer), the nodes are the solid features and the fibrils are not the solid features. Indeed, in some embodiments, the fibrils may be removed, leaving only the nodes in the mitigation layer. In embodiments where the nodes within the mitigation layer are the solid features, those nodes which are bonded to the cell impermeable layer are bonded solid features. In at least one embodiment, the mitigation layer is formed of an expanded polytetrafluoroethylene (ePTFE) membrane having a node and fibril microstructure.

The solid features of the mitigation layer do not negatively impact the overall diffusion resistance of the biocompatible membrane composite for applications that require a rapid time course of diffusion. The solid features of the mitigation layer are of a sufficiently small size such that they do not interfere with the amount of porous area needed for diffusion across the cell impermeable layer. Also, the thickness of the mitigation layer is sufficiently thin so as to maximize mass transport of oxygen and nutrients to encapsulated cells from the interstitium during the acute period post implantation. The space between the solid features are sufficiently open to allow for easy and rapid penetration/integration of host tissue up to the cell impermeable layer (i.e., tight layer) to decrease the duration of the acute period. “Acute period” is defined herein as the time period prior to host cell/vascular infiltration.

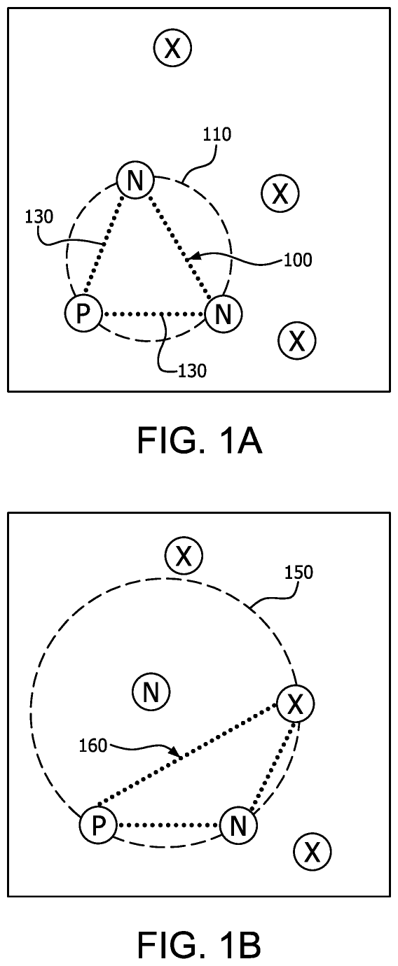

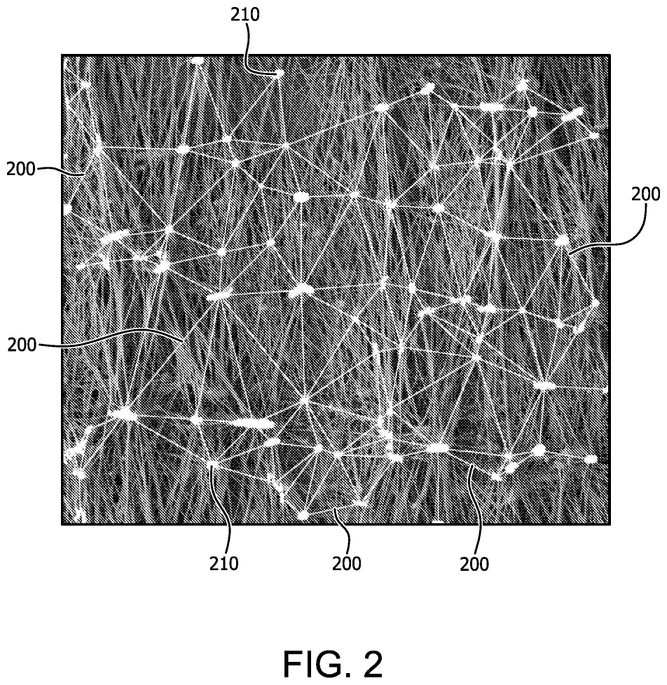

The solid feature spacing of the majority of solid features adjacent to the cell impermeable layer is less than about 50 microns, less than about 40 microns, less than about 30 microns, less than about 20 microns, or less than about 10 microns. As used herein, the term “majority” is meant to describe an amount over half (i.e., greater than 50%) of the measured values for the parameter being measured. In some embodiments, the majority of the solid feature spacing may range from about 5 microns to about 50 microns, from about 5 microns to about 45 microns, from about 10 microns to about 40 microns, from about 10 microns to about 35 microns, or from about 15 microns to about 35 microns. The phrase “solid feature spacing” is defined herein as the straight-line distance between two neighboring solid features. In this disclosure, solid features are considered neighboring if their centroids represent the corners of a triangle whose circumcircle has an empty interior. As shown pictorially in FIG. 1A, the designated solid feature (P) is connected to neighboring solid features (N) to form a triangle 100 where the circumcircle 110 contains no solid features within. Solid features (X) designate the solid features that are not neighboring solid features. Thus, in the instance depicted in FIG. 1A, the solid feature spacing 130 is the straight distance between the designated solid features (P), (N). In contrast, the circumcircle 150 shown in FIG. 1B drawn from the triangle 160 contains therein a solid feature (N), and as such, cannot be utilized to determine the solid feature spacing in the mitigation layer (or the vascularization layer). FIG. 2 is a scanning electron micrograph depicting measured distances, e.g., the white lines 200 between the solid features 210 (white shapes) in a mitigation layer formed of an expanded polytetrafluoroethylene (ePTFE) membrane.

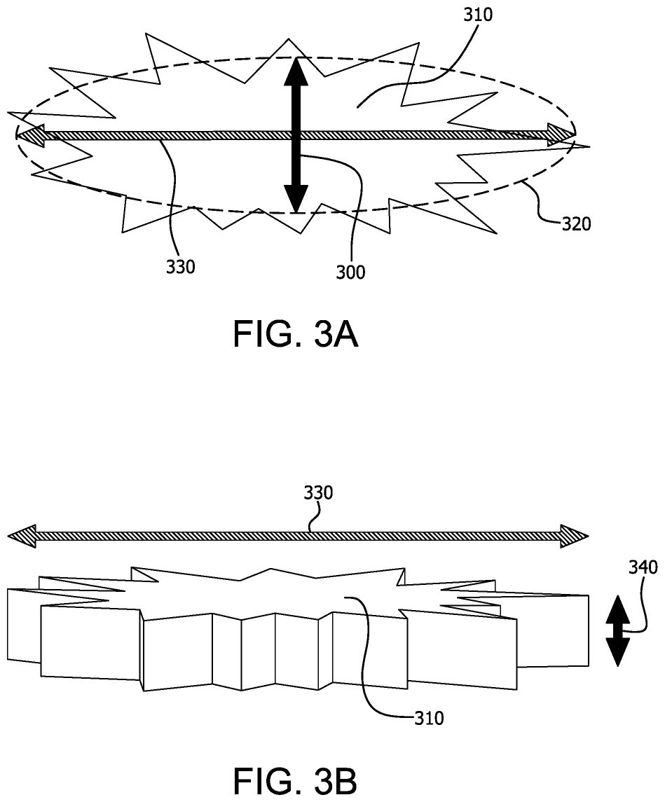

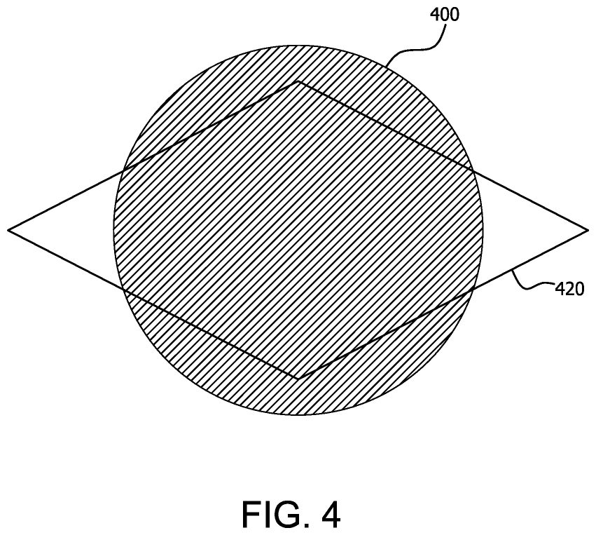

The solid features also include a representative minor axis, a representative major axis, and a solid feature depth. The representative minor axis of a solid feature is defined herein as the length of the minor axis of an ellipse fit to the solid feature where the ellipse has the same area, orientation, and centroid as the solid feature. The representative major axis of a solid feature is defined herein as the length of the major axis of an ellipse fit to the solid feature where the ellipse has the same area, orientation, and centroid as the solid feature. The major axis is greater than or equal to the minor axis in length. The minor and major axes of an ellipse 320 to fit the solid feature 310 is shown pictorially in FIG. 3A. The representative minor axis of the solid feature 310 is depicted by arrow 300, and the representative major axis of the solid feature 310 is depicted by arrow 330. The representative minor axis and representative major axis of a layer are the respective median values of all measured representative minor axes and representative major axes of features in the layer. A majority of the solid features has a minor axis that range in size from about 3 microns to about 20 microns, from about 3 microns to about 15 microns, or from about 3 microns to about 10 microns. The solid feature depth is the length of the projection of the solid feature in the axis perpendicular to the surface of the layer (e.g., mitigation layer or vascularization layer). The solid feature depth of the solid feature 310 is shown pictorially in FIG. 3B. The depth of the solid feature 310 is depicted by line 340. In at least one embodiment, the depth of the solid features is equal to or less than the thickness of the mitigation layer. The solid feature depth of a layer is the median value of all measured solid feature depths in the layer. In at least one embodiment, a majority of at least two of the mitigation layer representative minor axis, average representative major axis, and average solid feature depth is greater than 5 microns.

In embodiments where the solid features are interconnected by fibrils or fibers, the boundary connecting the solid features creates a pore. It is necessary that these pores are open enough to allow rapid cellular ingrowth and vascularization and not create a resistance to mass transport of oxygen and nutrients. The pore effective diameter is measured by quantitative image analysis (QIA) and performed on a scanning electron micrograph (SEM) image. The “effective diameter” of a pore is defined as the diameter of a circle that has an area equal to the measured area of the surface pore. This relationship is defined by the following equation:

$$Effective Diameter = 2 \times \sqrt{\frac{Area}{π}}$$

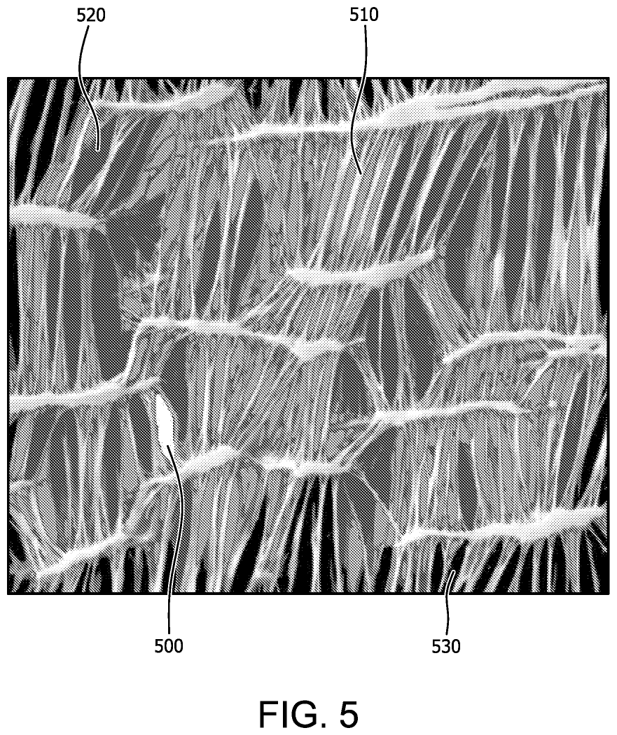

Turning to FIG. 4, the effective diameter is the diameter of the circle 400 depicted in FIG. 4 and the surface pore is designated by reference numeral 420. The total pore area of a surface is the sum of the area of all pores at that surface. The pore size of a layer is the effective diameter of the pore that defines the point where roughly half the total pore area consists of pores with diameters smaller than the pore size and half the total pore area consists of pores with diameters greater than or equal to the pore size. FIG. 5 illustrates a pore size 500 (white in color), pores smaller in size 510 (shown in light grey), and pores larger in size 520 (shown in dark grey). Pores that intersect with the edge of the image 530 were excluded from analysis and are shown in black

The pore size of the mitigation layer may range from about 1 micron to about 9 microns in effective diameter, from about 3 microns in effective diameter to about 9 microns in effective diameter, or from about 4 micron in effective diameter to about 9 microns in effective diameter as measured by quantitative image analysis (QIA) performed on an SEM image. Also, the mitigation layer has a thickness that is less than about 200 microns, less than about 290 microns, less than about 280 microns, less than about 270 microns, less than about 260 microns, less than about 200 microns, less than about 190 microns, less than about 180 microns, less than about 170 microns, less than about 160 microns, less than about 150 microns, less than about 140 microns, less than about 130 microns, less than about 120 microns, less than about 110 microns, less than about 100 microns, less than about 90 microns, less than about 80 microns, less than about 70 microns, or less than about 60 microns, less than 50 about microns, less than about 40 microns, less than about 30 microns, less than about 20 microns, or less than about 10 microns. The thickness of the mitigation layer may range from about 60 microns to about 200 microns, from about 60 microns to about 170 microns, from about 60 to about 150 microns, from about 60 microns to about 125 microns, from about 60 microns to about 100 microns, from about 3 microns to about 60 microns, from about 10 microns to about 50 microns, from about 10 microns to about 40 microns, or from about 15 microns to about 35 microns. In some embodiments, the mitigation layer has a porosity greater than about 60%. In other embodiments, the mitigation layer has a porosity greater than about 70%, greater than about 75%, greater than about 80%, or greater than about 85%. Additionally, the porosity of the mitigation layer may range from about 60% to about 90%, from about 70% to about 90%, from about 75% to about 90%, from about 80% to about 90%, or from about 80% to about 90%. In at least one embodiment, the porosity may be about 80%.

In some embodiments, the biocompatible membrane composite, including the cell impermeable layer, is perforated with discretely placed holes. The perforation size, number, and location can be selected to optimize cell function. As few as one (1) perforated hole may be present. The perforations are of a sufficient size to allow host vascular tissue (such as capillaries) to pass through the biocompatible membrane composite in order to support, for example, encapsulated pancreatic cell types. While the cell impermeable layer maintains its function as a microporous, immune isolation barrier in locations where no perforations are present, due to the discrete perforations where portions of the cell impermeable layer have been removed, the cell impermeable layer in its entirety is no longer cell impermeable because the discrete perforations allow vascular ingrowth and cellular contact from the host to pass through the biocompatible membrane composite. Because cell encapsulation device embodiments that contain a perforated cell impermeable layer allow for host immune cell contact with cells, the cells are no longer protected from immune rejection unless the host is immunocompromised or treated with immunosuppressant drugs.

An optional reinforcing component may be provided to the biocompatible membrane composite to minimize distortion in vivo so that the cell bed thickness is maintained (e.g., in an encapsulated device). This additional optional reinforcing component provides a stiffness to the biocompatible membrane composite that is greater than the biocompatible membrane composite itself to provide mechanical support. This optional reinforcing component could be continuous in nature or may be present in discrete regions on the biocompatible membrane composite, e.g., either patterned across the entire surface of the biocompatible membrane composite or located in specific locations such as around the perimeter of the biocompatible membrane composite. Non-limiting patterns suitable for the reinforcing component on the surface of the membrane composite include dots, straight lines, angled lines, curved lines, dotted lines, grids, etc. Patterns forming the reinforcing component may be used singly or in combination. In addition, the reinforcing component may be temporary in nature (e.g., formed of a bioabsorbable material) or permanent in nature (e.g., a polyethylene terephthalate (PET) mesh or Nitinol). As is understood by one of ordinary skill in the art, the impact of component stiffness depends not just on the stiffness of a single component, but also on the location and restraint of the reinforcing component in the final device form. For instance, the stiffness of a reinforcing component should be sufficient to minimize or prevent distortion of the cell encapsulation device in vivo. Additionally, the stiffness attributed to the reinforcing component should not be so great as to minimize tissue response due to compliance mismatch with surrounding tissue. Depending on the specific details of the design of the encapsulation device, the stiffness of the device may range from about 0.01 N/cm to about 5 N/cm, from about 0.05 N/cm to about 4 N/cm, from about 0.1 N/cm to about 3 N/cm, or from about 0.3 N/cm to about 2 N/cm. In some embodiments, an external reinforcing component may be used on one or both sides of the biocompatible membrane composite to achieve the desired device stiffness.

An external reinforcing component may have a stiffness greater than 0.01 N/cm when measured separate from the cell encapsulation device. The stiffness of an external reinforcing component may range from about 0.01 N/cm to about 3 N/cm, from about 0.05 N/cm to about 2 N/cm, from about 0.09 to about 1 N/cm. In some embodiments, an internal reinforcing component may be used achieve the desired stiffness of the cell encapsulation device. An internal reinforcing component may have a stiffness greater than about 0.05 N/cm when measured separately from the cell encapsulation device. The stiffness of an internal reinforcing component may range from about 0.05 N/cm to about 5 N/cm, from about 0.1 N/cm to about 3 N/cm, or from about 0.3N/cm to about 2 N/cm.

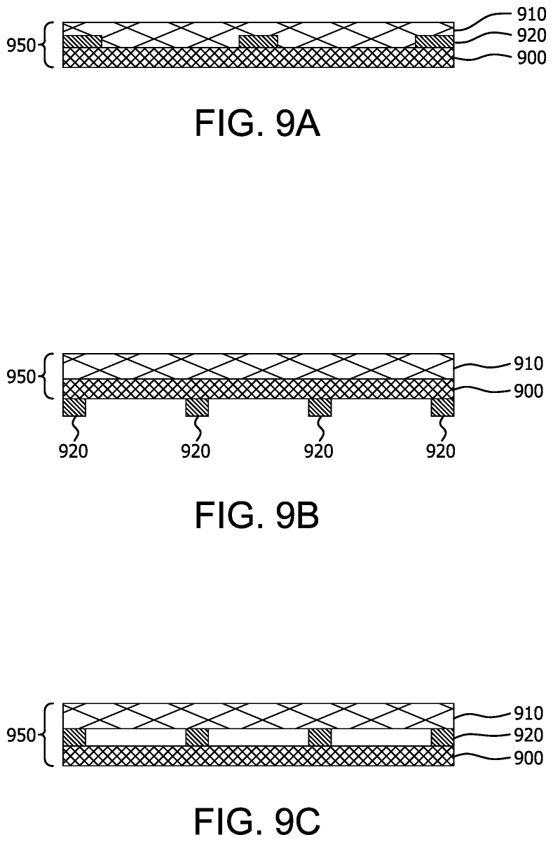

In at least one embodiment, the reinforcing component may be provided on the outer surface (e.g., farthest from the lumen of the cell encapsulation device) of the mitigation layer to strengthen the biocompatible membrane composite against environmental forces. This is one example of an external reinforcing component. In this orientation, the reinforcing component has a pore size sufficient to permit vascular ingrowth, and is therefore is also considered an “open” layer. Materials useful as the reinforcing component include materials that are significantly stiffer than the biocompatible membrane composite. Such materials include, but are not limited to, open mesh biomaterial textiles, woven textiles, non-woven textiles (e.g., collections of fibers or yarns), and fibrous matrices, either alone or in combination. In another embodiment, patterned grids, screens, strands and/or rods may be used as the reinforcing component. The reinforcing component may be positioned on the outer surface of the biocompatible membrane composite adjacent to the cell impermeable layer (see, e.g. FIG. 9B). This is an example of an internal reinforcing component In this orientation, the reinforcing component may be a cell impermeable and nutrient impermeable dense layer as long as there is sufficient spacing for cells to reside between the nutrient impermeable dense layer (i.e., reinforcing component) and the cell impermeable layer. Additionally, the reinforcing component may be oriented within the mitigation layer at discrete regions (see, e.g. FIG. 9A). In some embodiments, the reinforcing component may be positioned between the cell impermeable layer and the mitigation layer (see, e.g. FIG. 9C). It is to be appreciated that there may be more than one reinforcing component and the reinforcing component may be located externally to the biocompatible membrane composite, internally within the biocompatible membrane composite, both externally to and internally within the biocompatible membrane composite. Although not discussed in detail herein, it is to be appreciated that other layers (e.g. a vascularization layer, a mesh layer, a fabric layer, a reinforcing component, etc.) on or within the biocompatible membrane composite are not precluded from inclusion therein and are considered to be within the purview of this disclosure.

In at least one embodiment, the cell impermeable layer and the mitigation layer are bonded together by one or more biocompatible adhesive to form the biocompatible membrane composite. The adhesive may be applied to the surface of one or both of the cell impermeable layer and the mitigation layer in a manner to create a discrete or intimate bond between the layers. Non-limiting examples of suitable biocompatible adhesives include fluorinated ethylene propylene (FEP), a polycarbonate urethane, a thermoplastic fluoropolymer comprised of TFE and PAVE, EFEP (ethylene fluorinated ethylene propylene), PEBAX (a polyether amide), PVDF (poly vinylidene fluoride), CarbOSil® (absilicone polycarbonate urethane), Elasthane™ (a polyether urethane), PurSil® (a silicone polyether urethane), polyethylene, high density polyethylene (HDPE), ethylene chlorotetrafluoroethylene (ECTFE), perfluoroalkoxy (PFA), polypropylene, polyethylene terephthalate (PET), and combinations thereof. In one or more embodiment, the mitigation layer is intimately bonded to the cell impermeable layer. In other embodiments, the cell impermeable layer and the mitigation layer may be discretely bonded to each other. As used herein, the phrases “discrete bond” or “discretely bonded” are meant to include bonding or bonds in intentional patterns of points or lines or around a continuous perimeter of a defined region. In some embodiments, the cell impermeable layer and the mitigation layer are co-expanded as a composite. In yet another embodiment, the cell impermeable layer may, at least in part, be bound to the mitigation layer by bonded solid features, thereby creating a discrete bond between the cell impermeable layer and the mitigation layer. In certain intimately bonded embodiments, measured composite z-strengths are greater than 100 kPa. The measured composite z-strength may range from about 100 kPa to about 1300 kPa, from about 100 kPa to about 1100 kPa, from about 100 kPa to about 900 kPa, from about 100 kPa to about 700 kPa, from about 100 kPa to about 500 kPa, from about 100 kPa to about 300 kPa, or from about 100 kPa to about 200 kPa.

At least one of the cell impermeable layer and the mitigation layer may be formed of a polymer membrane or woven or non-woven collections of fibers or yarns, or fibrous matrices, either alone or in combination. Non-limiting examples of polymers that may be used one or both of the cell impermeable layer and the mitigation layer include, but are not limited to, alginate; cellulose acetate; polyalkylene glycols such as polyethylene glycol and polypropylene glycol; panvinyl polymers such as polyvinyl alcohol; chitosan; polyacrylates such as polyhydroxyethylmethacrylate; agarose; hydrolyzed polyacrylonitrile; polyacrylonitrile copolymers; polyvinyl acrylates such as polyethylene-co-acrylic acid, polyalkylenes such as polypropylene, polyethylene; polyvinylidene fluoride; fluorinated ethylene propylene (FEP); perfluoroalkoxy alkane (PFA); polyester sulfone (PES); polyurethanes; polyesters; and copolymers and combinations thereof. Examples of materials that may be used to form the mitigation layer include, but are not limited to, may include biocompatible textiles, including wovens and non-woven fabrics (e.g., a spunbound non-woven, melt blown fibrous materials, electrospun nanofibers, etc.), non-fluoropolymer membranes such as polyvinylidene difluoride (PVDF), nanofibers, polysulfones, polyethersulfones, polyarlysulfones, polyether ether ketone (PEEK), polyethylenes, polypropylenes, and polyimides. In exemplary embodiments, the vascularization layer is a spunbound polyester or an expanded polytetrafluoroethylene (ePTFE) membrane.

In some embodiments at least one of the mitigation layer or reinforcing component is formed of a non-woven fabric. There are numerous types of non-woven fabrics, each of which may vary in tightness of the weave and the thickness of the sheet. In one embodiment, the filament cross-section is trilobal. The non-woven fabric may be a bonded fabric, a formed fabric, or an engineered fabric that is manufactured by processes other than weaving or knitting. In some embodiments, the non-woven fabric is a porous, textile-like material, usually in flat sheet form, composed primarily or entirely of fibers, such as staple fibers assembled in a web, sheet, or batt. The structure of the non-woven fabric is based on the arrangement of, for example, staple fibers that are typically randomly arranged. In addition, non-woven fabrics can be created by a variety of techniques known in the textile industry. Various methods may create carded, wet laid, melt blown, spunbonded, or air laid non-wovens. Methods and substrates are described, for example, in U.S. Patent Publication No. 2010/0151575 to Colter, et al. In one embodiment the non-woven fabric is polytetrafluoroethylene (PTFE). In one embodiment the non-woven fabric is a spunbound polyester. The density of the non-woven fabric may be varied depending upon the processing conditions. In one embodiment the non-woven fabric is a spunbound polyester with a basis weight from about 0.40 to about 1.00 (oz/yd²) a nominal thickness from about 127 microns to about 228 microns and a fiber diameter from about 0.5 microns to about 26 microns. In one embodiment, the filament cross-section is trilobal. In some embodiments, the non-woven fabrics are bioabsorbable.

In some embodiments, the polymer(s) forming the polymer membrane of the cell impermeable layer and/or the mitigation layer is fibrillatable. Fibrillatable, as used herein, refers to the ability to introduce fibrils to a polymer membrane, such as, but not limited to, converting portions of the solid features into fibrils. For example, the fibrils are solid elements that span the gaps between the solid features. Fibrils are generally not resistant to deformation upon exposure to environmental forces, and are therefore deformable. The majority of deformable fibrils present in one of the layers of the biocompatible membrane composite may have a diameter less than about 2 microns, less than about 1 micron, less than about 0.75 microns, less than about 0.50 microns, or less than about 0.25 microns. In some embodiments, the majority of deformable fibrils may have a diameter from about 0.25 microns to about 2.0 microns, from about 0.5 microns to about 2 microns, or from about 0.75 microns to about 2 microns.

Non-limiting examples of fibrillatable polymers that may be used to form one or more of the cell impermeable layer and the mitigation layer include, but are not limited to, tetrafluoroethylene (TFE) polymers such as polytetrafluoroethylene (PTFE), expanded PTFE (ePTFE), modified PTFE, TFE copolymers, polyvinylidene fluoride (PVDF), poly (p-xylylene) (ePPX) as taught in U.S. Patent Publication No. 2016/0032069 to Sbriglia, porous ultra-high molecular weight polyethylene (eUHMWPE) as taught in U.S. Pat. No. 9,926,416 to Sbriglia, porous ethylene tetrafluoroethylene (eETFE) as taught in U.S. Pat. No. 9,932,429 to Sbriglia, and porous vinylidene fluoride-co-tetrafluoroethylene or trifluoroethylene [VDF-co-(TFE or TrFE)] polymers as taught in U.S. Pat. No. 9,441,088 to Sbriglia and combinations thereof.

In some embodiments, the fibrillatable polymer is a fluoropolymer membrane such as an expanded polytetrafluoroethylene (ePTFE) membrane. Expanded polytetrafluoroethylene (ePTFE) membranes have a node and fibril microstructure where the nodes are interconnected by the fibrils and the pores are the space located between the nodes and fibrils throughout the membrane. As used herein, the term “node” is meant to denote a solid feature consisting largely of polymer material. When deformable fibrils are present, these nodes reside at the junction of multiple fibrils. In some embodiments, the fibrils may be removed from the membrane, such as, for example, by plasma etching.

In at least one embodiment, an expanded polytetrafluoroethylene membrane is used in one or both of the cell impermeable membrane layer and the mitigation layer. Expanded polytetrafluoroethylene membranes such as, but not limited to, those prepared in accordance with the methods described in U.S. Pat. No. 3,953,566 to Gore, U.S. Pat. No. 7,306,729 to Bacino et al., U.S. Pat. No. 5,476,589 to Bacino, WO 94/13469 to Bacino, U.S. Pat. No. 5,814,405 to Branca et al. or U.S. Pat. No. 5,183,545 to Branca et al. may be used herein. In some embodiments, one or both of the cell impermeable layer and the mitigation layer is formed of a fluoropolymer membrane, such as, but not limited to, an expanded polytetrafluoroethylene (ePTFE) membrane, a modified expanded polytetrafluoroethylene membrane, a tetrafluoroethylene (TFE) copolymer membrane, a polyvinylidene fluoride (PVDF) membrane, or a fluorinated ethylene propylene (FEP) membrane.

In some embodiments, it may be desirable for the reinforcing component and/or an additional layer (e.g. a vascularization layer, reinforcing component, a mesh layer, a fabric layer, etc.) to be non-permeant (e.g., biodegradable). In such an instance, a biodegradable material may be used to form the reinforcing component. Suitable examples of biodegradable materials include, but are not limited to, polyglycolide:trimethylene carbonate (PGA:TMC), polyalphahydroxy acid such as polylactic acid, polyglycolic acid, poly (glycolide), and poly(lactide-co-caprolactone), poly(caprolactone), poly(carbonates), poly(dioxanone), poly (hydroxybutyrates), poly(hydroxyvalerates), poly (hydroxybutyrates-co-valerates), expanded polyparaxylylene (ePLLA), such as is taught in U.S. Patent Publication No. 2016/0032069 to Sbriglia, and copolymers and blends thereof. Alternatively, the mitigation layer may be coated with a bio-absorbable material or a bio-absorbable material may be incorporated into or onto the mitigation layer in the form of a powder. Coated materials may promote infection site reduction, vascularization, and favorable type 1 collagen deposition.

The biocompatible membrane composite may have at least partially thereon a surface coating, such as a Zwitterion non-fouling coating, a hydrophilic coating, or a CBAS®/Heparin coating (commercially available from W.L. Gore & Associates, Inc.). The surface coating may also or alternatively contain antimicrobial agents; antibodies (e.g., anti-CD 47 antibodies (anti-fibrotic)); pharmaceuticals; biologically active molecules (e.g., stimulators of vascularization such as FGF, VEGF, endoglin, PDGF, angiopoetins, and integrins; anti-fibrotic such as TGFb inhibitors; sirolimus, CSF1R inhibitors; anti-inflammatory/immune modulators such as CXCL12, and corticosteroids) anti CD 47 antibodies (anti-fibrotic), and combinations thereof.

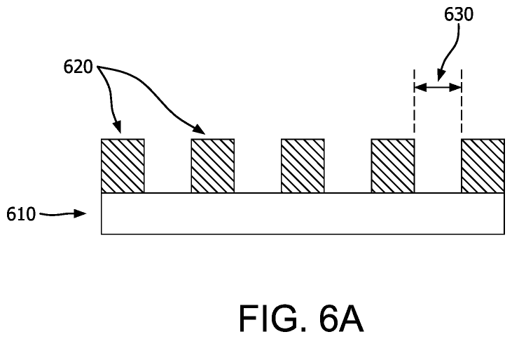

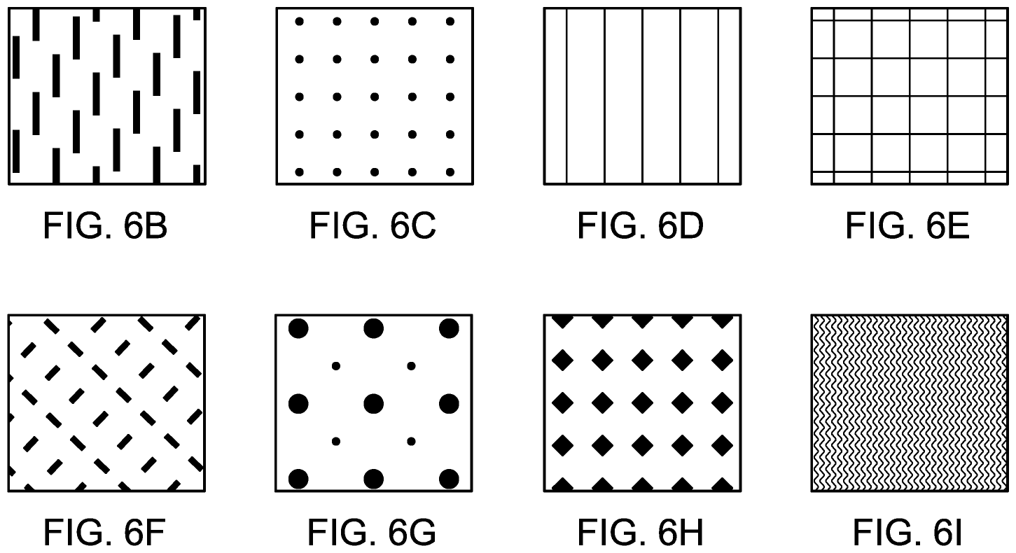

In some embodiments, the solid features of the mitigation layer may be formed by microlithography, micro-molding, machining, selectively depositing, or printing (or otherwise laying down) a polymer (e.g., thermoplastic) onto a cell impermeable layer to form at least a part of a solid feature. Any conventional printing technique such as transfer coating, screen printing, gravure printing, ink-jet printing, patterned imbibing, and knife coating may be utilized to place the thermoplastic polymer onto the cell impermeable layer. FIG. 6A illustrates a thermoplastic polymer in the form of solid features 620 positioned on a cell impermeable layer 610 (after printing is complete), where the solid features 620 have a feature spacing 630. Non-limiting examples of geometries for forming the solid features include, but are not limited to, dashed lines (see FIG. 6B), dots and/or dotted lines (see FIGS. 6C, 6G), geometric shapes (see FIG. 6H), straight lines (see FIG. 6D), angled lines (see FIG. 6F), curved lines (see FIG. 6I), grids (see FIG. 6E), and combinations thereof.

Materials used to form the solid features of the mitigation layer include, but are not limited to, thermoplastics, polyurethane, polypropylene, silicones, rubbers, epoxies, polyethylene, polyether amide, polyetheretherketone, polyphenylsulfone, polysulfone, silicone polycarbonate urethane, polyether urethane, polycarbonate urethane, silicone polyether urethane, polyester, polyester terephthalate, melt-processable fluoropolymers, such as, for example, fluorinated ethylene propylene (FEP), tetrafluoroethylene-(perfluoroalkyl) vinyl ether (PFA), an alternating copolymer of ethylene and tetrafluoroethylene (ETFE), a terpolymer of tetrafluoroethylene (TFE), hexafluoropropylene (HFP) and vinylidene fluoride (THV), polyvinylidene fluoride (PVDF), and combinations thereof. In some embodiments, polytetrafluoroethylene may be used to form the pattern features. In further embodiments, the solid features may be separately formed and may be adhered to the surface of the cell impermeable layer (not illustrated).

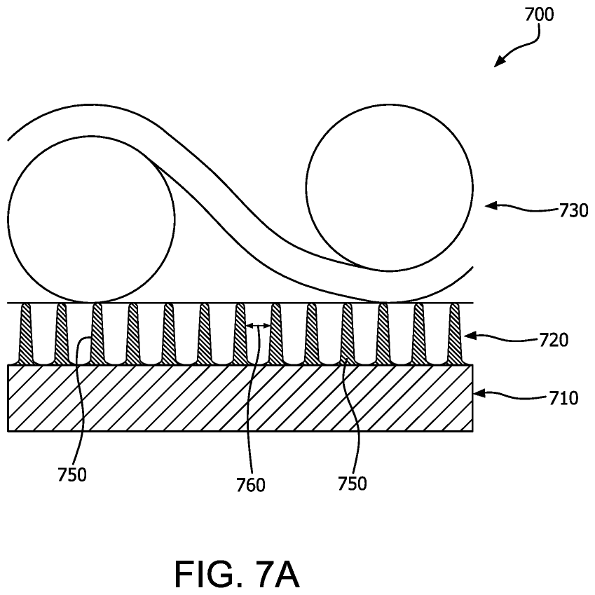

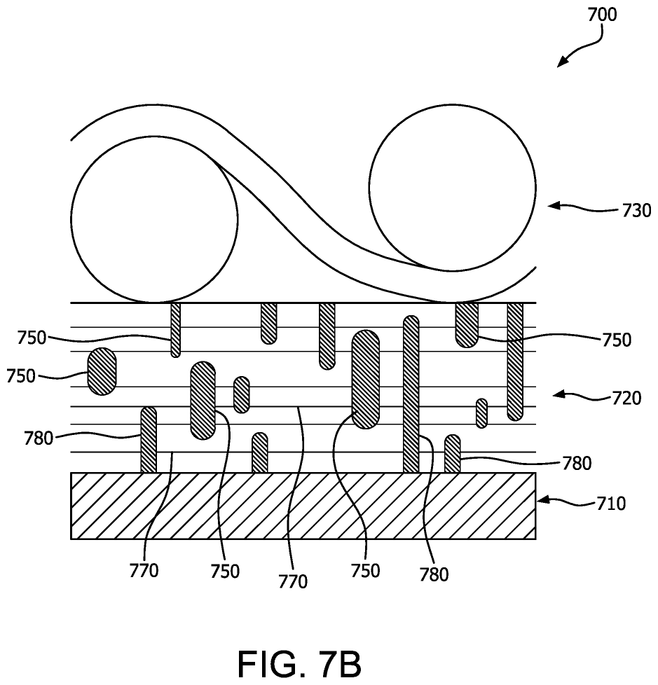

The biocompatible membrane composite 700 depicted in FIG. 7A includes a cell impermeable layer 710, a mitigation layer 720, and an optional reinforcement component 730. In the depicted embodiment, the solid features 750 are bonded to the surface of the cell impermeable layer 710 to form the mitigation layer 720. The solid features 750 are depicted in FIG. 7A as being essentially the same height and width and extending between the cell impermeable layer 710 and the optional reinforcement layer 730, although it is to be appreciated that this is an example and the solid features 750 may vary in height and/or width. The distance between solid features 750 is the solid feature spacing 760, and may, in some instances, vary between the various solid features 750.

FIG. 7B is another biocompatible composite 700 that includes a cell impermeable layer 710, a mitigation layer 720, and an optional reinforcement component 730. In the depicted embodiment, the solid features 750, 780 are nodes that differ in height and width, and may or may not extend the distance between the cell impermeable layer 710 and the optional reinforcement layer 730. The solid features 750, 780 are connected by fibrils 770. In FIG. 7B, the majority of the solid feature depth is less than the thickness of the mitigation layer 720. The solid features 780 are bonded solid features.

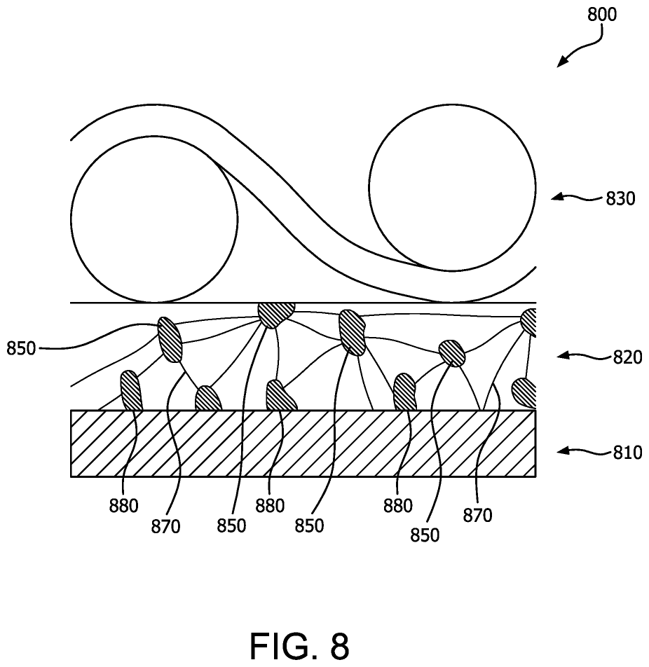

Turning to FIG. 8, a biocompatible membrane composite is depicted that contains a cell impermeable layer 810, a mitigation layer 820, and an optional reinforcement layer 830. In this embodiment, solid features 850, 880 within the mitigation layer 820 are the nodes of a mitigation layer 820 that is formed within an expanded polytetrafluoroethylene membrane. The nodes 850, 880 are interconnected by fibrils 870. Nodes 850 are positioned within the mitigation layer 820. Nodes 880 are not only positioned within the mitigation layer 820, but also are in contact with, and are intimately bonded to, the cell impermeable layer 810.

As discussed above, the reinforcing component may be oriented within or between the layers of the biocompatible membrane composite at discrete regions. In one non-limiting embodiment shown in FIG. 9A, the reinforcing component 920 is formed as discrete regions on the inner surface of the cell impermeable layer 900 and are positioned within the mitigation layer 910 in the biocompatible membrane composite 950. In the embodiment depicted in FIG. 9B, the reinforcing component 920 is positioned on the cell impermeable layer 900 on a side opposing the mitigation layer 910 and is external to the biocompatible membrane composite 950. In yet another non-limiting embodiment depicted in FIG. 9C, the reinforcing component 920 is positioned between the cell impermeable layer 900 and the mitigation layer 910 in to the biocompatible membrane composite 950.

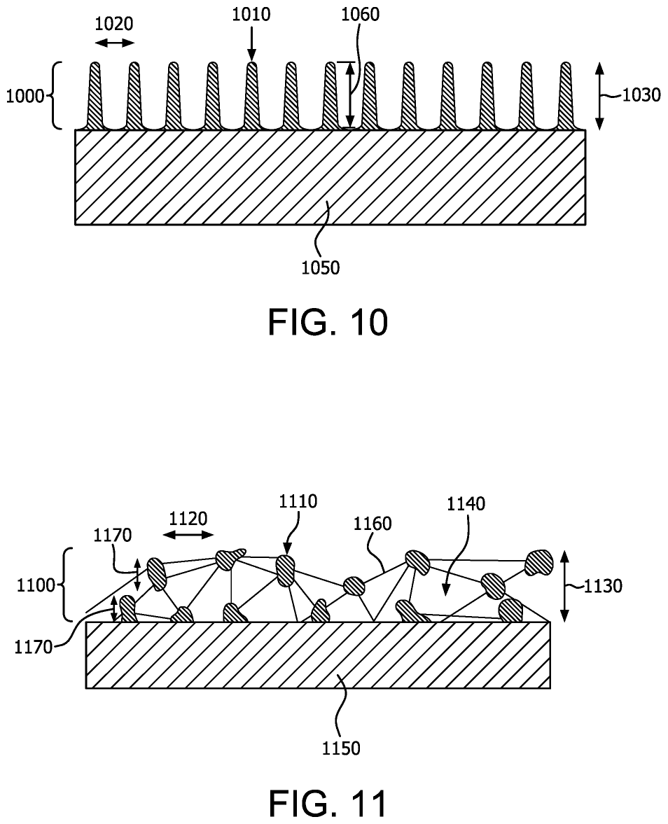

Turning to FIG. 10, the mitigation layer 1000 may be formed by placing a polymer in a pattern (as described above) which is characterized by one or more of the following: the solid feature size (i.e., minor axis) 1010, solid feature spacing 1020, thickness 1030, the absence of fibrils and/or the pore size as measured by quantitative image analysis (QIA) performed on an SEM image as depicted generally in FIG. 10. A cell impermeable layer 1050 is shown for reference only.

FIG. 11 depicts a mitigation layer 1100 that is formed of a polymer having a node and fibril microstructure that is characterized by one or more of the following: the solid feature size (i.e., minor axis) 1110, solid feature spacing 1120, solid feature depth 1170, thickness 1130, the presence of fibrils 1160 and/or the pore size (as measured by quantitative image analysis (QIA) performed on an SEM image) 1140 as depicted generally in FIG. 11. A cell impermeable layer 1150 is shown for reference only.

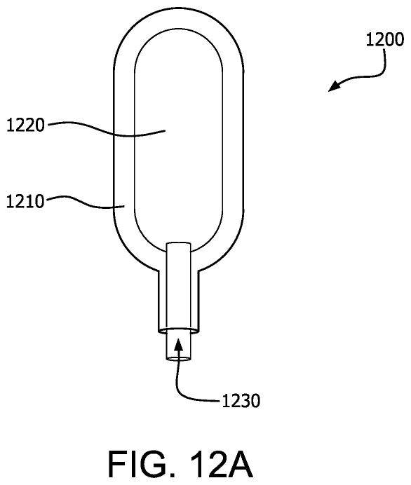

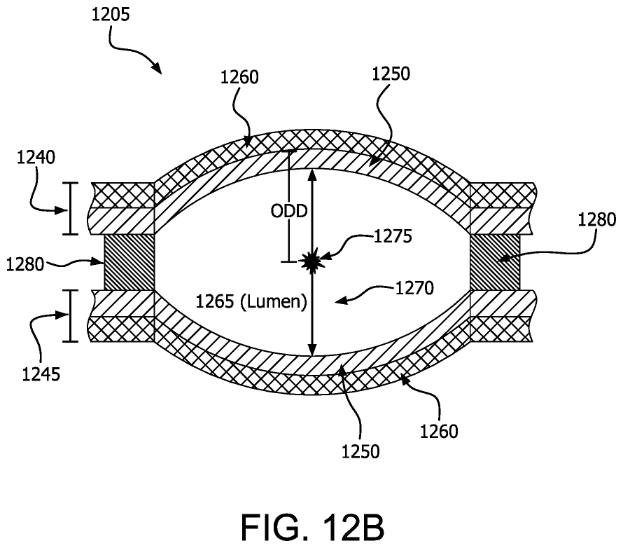

The biocompatible membrane composite can be manufactured into various forms including, but not limited to, a cell encapsulation device, a housing, a chamber, a pouch, a tube, or a cover. In one embodiment, the biocompatible membrane composite forms a cell encapsulating device as illustrated in FIG. 12A. FIG. 12A is a top view of an exemplary cell encapsulating device 1200 formed of two layers of the biocompatible membrane composite that are sealed along a portion of their peripheries 1210. Only the external layer (e.g., side of the biocompatible membrane composite that is in contact with host tissue when implanted) of the biocompatible membrane composite 1220 is shown in FIG. 12A. The cell encapsulating device 1200 includes an internal chamber, also known as a lumen (not shown) for containing cells of interest and a fill tube 1230 that extends into the internal chamber and is in fluid communication therewith to place the cells of interest within the lumen.

The cell encapsulation devices described herein each have an oxygen diffusion distance that is sufficient for the survival of the encapsulated cells when implanted in vivo. The term “oxygen diffusion distance” (ODD) is meant to define the distance from a hypothetical cell located in the most interior portion of the lumen to the hypothetical closest source of vascularization located on the outer side of the closest cell impermeable layer. Since this measure is most relevant when the encapsulation device is implanted in vivo and the mitigation layer enables the formation of blood vessels at the cell impermeable interface, the oxygen diffusion distance is measured in vitro when the device is pressurized to represent a realistic state when internal pressure is generated from the growth of the encapsulated cells inside the lumen. The range of in vitro pressures to use to measure oxygen diffusion distance can range from 0.5 to 5 psi of internal pressure. The oxygen diffusion distance can be measured at various locations across the active surface area of the cell encapsulation device. The term “active surface area” as used herein refers to the area bordering the open lumen space that can facilitate mass transport of nutrients (i.e., microporous) and that can be filled with a biological entity or cells. The maximum diffusion distance represents the greatest oxygen diffusion distance of all possible hypothetical cells within the lumen to the closest potential source of vascularization. The maximum oxygen diffusion distance (ODD) is defined herein as the point of greatest deflection of the membrane composite when pressurized. The oxygen diffusion distance can also be assessed relative to the proportion of the total active surface area of the cell encapsulation device. The majority oxygen diffusion distance as used herein represents the oxygen diffusion distance of the hypothetical most interior cell in the lumen across the majority of the active surface area of the device (>50%). To maximize the viability and productivity of the encapsulated cells, either the maximum diffusion distance needs to be kept to a minimum distance or the majority oxygen diffusion distance of the active surface area needs to be kept to a minimum distance. In one embodiment the oxygen diffusion distance remains consistent across the entire active surface area of the device such that there is a minimal difference between the maximum oxygen diffusion distance and the majority oxygen diffusion distance.

As one example, FIG. 12B depicts a cross section of a cell encapsulation device similar to that shown in FIG. 12A. The cell encapsulation device 1205 contains a biocompatible membrane composite 1240 and a biocompatible membrane composite 1245 with a lumen 1265 therebetween. Each biocompatible membrane composite 1240, 1245 is formed of a cell impermeable layer (a first layer) 1250 and a mitigation layer (second layer) 1260 and is sealed around its periphery 1280. It is to be appreciated that the biocompatible membrane composites 1240, 1245 forming the device 1205 may contain the same or different cell impermeable layers and/or mitigation layers. Optional layers such as a reinforcement component (third layer) (not shown) may be included external to the mitigation layer 1260 and may at least partially surround or surround the encapsulation device. It is also to be appreciated that, although not depicted, the encapsulation device 1205 has a filling tube to inject or otherwise insert cells of interest. Shown schematically in FIG. 12B, the maximum oxygen diffusion distance (ODD) is represented since it depicts the maximum deflection of the membrane composite and thereby the greatest distance of all possible encapsulated cells 1275 to the nearest possible blood vessel that could be formed on the outside of the cell impermeable layer 1250. In a device where there is no internal reinforcing component or other such device within the lumen to separate separating two layers of opposing membrane composites joined at perimeter seal, the ODD may be calculated by measuring the total expansion of the lumen when pressurized as shown in the distance of arrow 1270, dividing it by 2 (two), and adding the thickness of the cell impermeable layer. In some embodiments, the maximum oxygen diffusion distance (ODD) is from about 7 microns to about 500 microns, from about 10 microns to about 400 microns, from about 25 microns to about 350 microns, from about 50 microns to about 300 microns, from about 50 microns to about 250 microns, from about 75 microns to about 250 microns, from about 50 microns to about 200 microns, from 75 microns to about 200 microns, from about 25 microns to about 200 microns, from about 10 microns to about 200 microns or from about 7 microns to about 100 microns. In a preferred embodiment, the ODD is about 300 microns (600 microns total lumen thickness), about 200 microns (400 microns total lumen thickness), about 150 microns (300 microns total lumen thickness), or about 100 microns (200 microns total thickness), assuming that the cell impermeable membrane (a first layer) has thicknesses (or negligible thickness) and dimensions as described herein.

Additionally, instead of only measuring at the point of maximum deflection, the oxygen diffusion distance can also be measured at multiple locations across the active surface area of the cell encapsulation device to assess the oxygen diffusion distance across the majority of the active surface area (herein “majority oxygen diffusion distance”). In some embodiments, the majority oxygen diffusion distance may be less than 300 microns. In some embodiments, the majority oxygen diffusion distance is from about 7 microns to about 300 microns, from about 7 microns to about 250 microns, from about 7 microns to about 200 microns, from about 7 microns to about 150 microns, from about 7 microns to about 100 microns, from about 7 microns to about 75 microns, from about 7 microns to about 50 microns, or from about 25 microns to about 250 microns, from about 25 microns to about 200 microns, or from about 25 microns to about 150 microns.

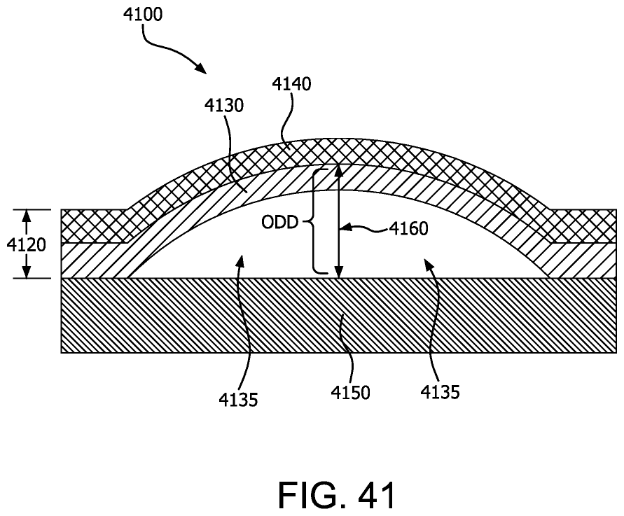

Another cell encapsulation device that may be formed by the biocompatible membrane composites is a planar device that includes an internal reinforcing component that is planar or substantially planar, is nutrient impermeable, and bisects the cell encapsulation device across the perimeter seal in the thickness direction into two (or more) individual lumen spaces (e.g., multiple lumen spaces) each bordered by a single biocompatible membrane composite. The internal reinforcing component divides the lumen into two portions. In at least one embodiment, the internal planar insert is centrally located or substantially centrally located and divides the lumen substantially in half. “Substantially in half” as used herein is meant to denote that the lumen is divided in half with equal portions on both sides or nearly in half where one half may be slightly larger than the other half. A portion of a planar device 4100 is schematically illustrated in FIG. 41 in cross-section. As shown, the biocompatible membrane composite 4120 contains a cell impermeable layer 4130 and a mitigation layer 4140. The internal reinforcing component 4150 divides the lumen 4135 into two portions (only one portion of the lumen is 4135 depicted in FIG. 41).

As with the other cell encapsulation devices described herein, this cell encapsulation device has an oxygen diffusion distance that is sufficient for the survival of cells. In such an embodiment, the maximum oxygen diffusion distance (ODD) is the distance from the internal reinforcing component 4150 (e.g., planar insert) which represents the location of the hypothetical most interior cell to the external side of the cell impermeable layer 4130 at the location of maximum deflection of the lumen as depicted by the bracketed area ODD and illustrated by arrow 4160.

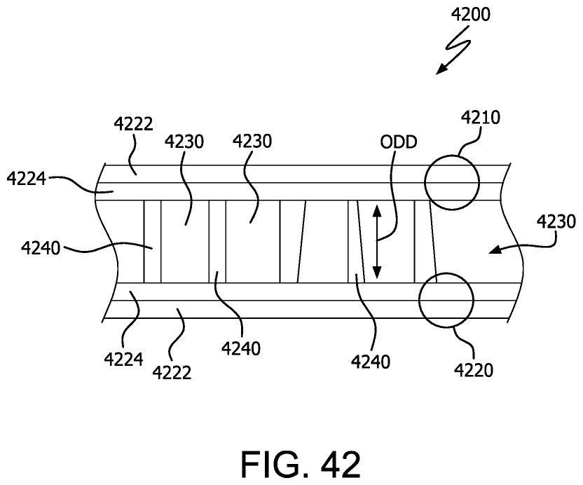

The cell encapsulation device maintains an optimal oxygen diffusion distance through either the inherent device construction, the use of reinforcing components, or the use of other lumen control mechanisms (as shown in FIGS. 42-46 and 48). The thickness of the lumen can be controlled in numerous ways. In one embodiment, the cell encapsulation device may be formed of two biocompatible membrane composites in which the cell impermeable layers face each other and the periphery of the membrane composites are sealed (e.g., welded) or bonded together, similar to the encapsulation devices shown in FIG. 12B and FIG. 41. However, unlike embodiments shown in FIG. 12B and FIG. 41, structural spacers such as polymeric pillars or printed structures may be located within the lumen and maintain a desired thickness of the lumen. Turning to FIG. 42, a schematic illustration of the lumen of such an encapsulation device may be seen. As shown, the cell encapsulation device 4200 includes a first biocompatible membrane composite 4210, a second biocompatible membrane composite 4220, a lumen 4230 positioned between the cell impermeable layers 4224 of the biocompatible membrane composites 4210, 4220, and structural spacers 4240 disposed within the lumen 4230 to separate the biocompatible membrane composites 4210, 4220. The structural spacers 4240 maintain a distance between the biocompatible membrane composites 4210, 4220 and thus the oxygen diffusion distance (ODD) is consistent through the active area of cell encapsulation device 4200 such that the maximum ODD is similar to the majority ODD. The mitigation layer 4222 is positioned as an external surface of the cell encapsulation device 4200, although this does not preclude the use of external reinforcements (e.g., a mesh) and such embodiments are considered to be within the purview of this disclosure. Further descriptions of cell encapsulation devices containing structural spacers can be found in U.S. Patent Publication No. 2018/0125632 to Cully, et al.

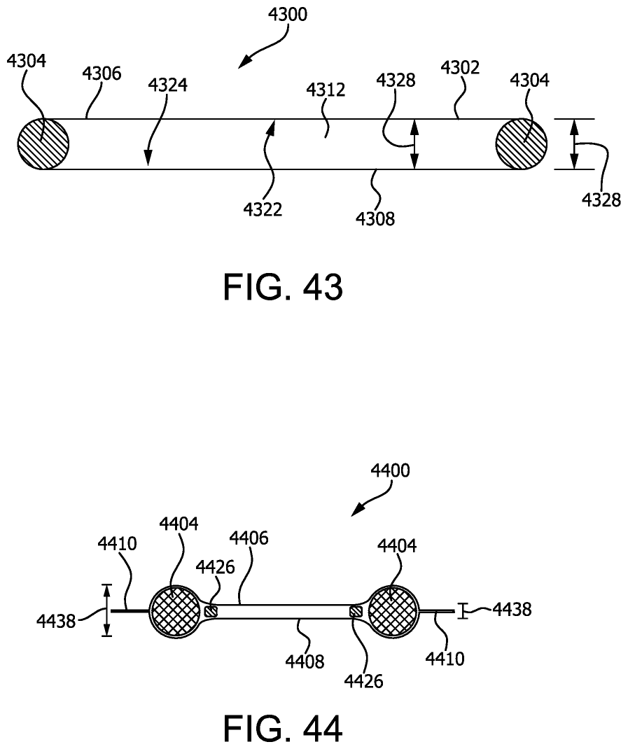

Another form of lumen control to optimize the oxygen diffusion distance is through the use of a tensioning member or tensioning members that exert opposing lateral forces away from the lumen. In one embodiment shown in FIG. 43, the cell encapsulation device 4300 is formed as an encapsulating pouch 4302 in a tubular shape and includes a first biocompatible membrane composite 4306, a second biocompatible membrane composite 4308, and a lumen 4312. A filling tube (not shown) can extend through the cell encapsulating pouch 4302 and can be in fluid communication with the lumen 4312. The first and second biocompatible membrane composites 4306, 4308 are sealed at their peripheries. A tensioning member 4304 is disposed within the lumen 4312, contacts at least two opposing portions of the cell encapsulating pouch 4302, and exerts tension on the first and second biocompatible membrane composites 4306, 4308. The lumen 4312 lies between the first and second biocompatible membrane composites 4306, 4308 and inwardly from the tensioning member 4304. The lumen 4312 has a thickness 4328 that is a distance from the innermost portion of the first biocompatible membrane 4306 to the innermost portion of the second biocompatible membrane 4308 and is defined by the thickness 4338 of the tensioning member 4304. In this embodiment, tension on the cell encapsulating pouch 4302 provided by the tensioning member 4304 impedes the collapsing or ballooning of the lumen 4312 and thus maintains the thickness 4328 defined by the tensioning member 4304. As a result, maximum and majority oxygen diffusion distance (ODD) is substantially the same across the cell encapsulating device 4300. It is to be appreciated that FIG. 43 does not illustrate any optional components, such as point bonds, structural spacers, a cell displacing core, or another structural element that may be disposed within the interior volume, but such embodiments are considered to be within the purview of this disclosure.

Another cell encapsulation device that controls the thickness of the lumen, and thus the oxygen diffusion distance, though the use of a tensioning member is shown in FIG. 44. FIG. 44 shows a cell encapsulating device 4400 includes a first biocompatible membrane composite 4406 and a second biocompatible membrane composite 4408 sealed along their peripheries 4410. The tensioning member 4404 is disposed within the lumen 4420, contacts at least two opposing portions of the cell encapsulating device 4400, and exerts tension on the first and second biocompatible membrane composites 4406, 4408. The lumen 4420 lies between the first and second membrane campsites 4406, 4408 and inwardly from weld spacers 4426. In this embodiment, the lumen thickness 4428 is defined by the thickness of the weld spacers 4426, and is independent from the thickness 4438 of the tensioning member 4404 because the weld spacers 4426 pinch the first and second biocompatible membrane composites 4406, 4408 together inwardly from the tensioning member 4404, and the thickness 4428 of the lumen 4420 is the thickness of the weld spacers 4426. Thus, in the embodiment illustrated in FIG. 44, the lumen thickness 4428 is less than the thickness 4438 of the tensioning member 4404. Alternatively, the weld spacers 4426 could have a thickness equal to or greater than the thickness 4438 of the tensioning member 4404, and in those embodiments, the thickness 4428 of the lumen 4420 would be equal to or greater than the thickness 4438 of the tensioning member 4404. Tension on the cell encapsulation device 4400 provided by the tensioning member 4404 hinders collapsing or ballooning of the lumen 4420 and thus maintains the thickness defined by the weld spacers 4426, and thus maintains the oxygen diffusion distance at a desired distance through lumen control.

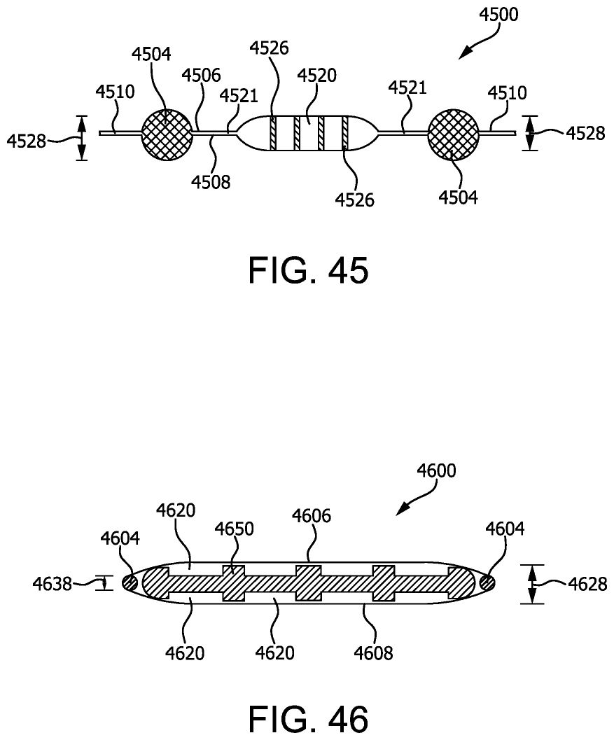

In yet another cell encapsulation device similar to that described with respect to FIG. 42, the cell encapsulating device 4500 shown in FIG. 45 is also formed from two separate membrane composites 4506, 4508 that are sealed along at least a portion of their peripheries 4510. Tensioning member 4504 is disposed between the first and second membrane composites 4506, 4508, contacts at least two opposing portions of the cell encapsulating device 4500, and exerts tension on the first and second biocompatible membrane composites 4506, 4508. However, instead of weld spacers as discussed above, the cell encapsulation device 4500 includes a seal 4521 that bonds the first and second biocompatible membrane composites 4506, 4508 to each other inwardly from tensioning member 4504. Inward from the seal 4521, structural spacers 4526 are positioned to separate the first and second membrane composites 4506, 4508, forming a lumen 4520 in the portion of the interior volume that is not occupied by the tensioning member 4504 or structural spacers 4526. In the embodiment depicted in FIG. 45, the thickness 4528 of the lumen 4520 is determined by the height of the structural spacers 4526. The thickness 4538 of the tensioning member 4504 is greater than the thickness 4528 of the lumen 4520. The tension on the cell encapsulating device 4500 provided by the tensioning member 4504 impedes collapsing or ballooning of the lumen 4520 and thus maintains the thickness defined by the structural spacers 4526 as well as the oxygen diffusion distance.

In yet another encapsulation device, the oxygen diffusion distance (ODD) is optimized by controlling the combined effect of the spacing between the perimeter seals of the encapsulation device and the stiffness of the external reinforcing component. Shorter distances between perimeter welds or discrete weld points within the lumen to either an internal reinforcing component or structural spacers between two biocompatible membrane composite layers decreases the amount of deflection possible between these welded locations, which better controls the ODD. As weld spacing is adjusted to increase or decrease the lumen length, it may also be necessary to adjust the device design to increase or decrease the lumen width to accommodate equivalent lumen volume capacities. The amount of deflection of the biocompatible membrane composites and resulting oxygen diffusion distance will be dependent on the presence and stiffness of a reinforcing component on the external side of the encapsulation device. Stiffer reinforcing components provide for less deflection of the membrane composites at equal spacing between welded locations. Non-limiting examples of external reinforcing components include textiles such as woven meshes and non-wovens formed of polymeric or metal strands, polymeric or metal spars or ribs, clamps, cages, fibers, strands, etc. In exemplary embodiments, the stiffness of the external reinforcing component is greater than 0.01 N/cm. In one embodiment, the stiffness of the external reinforcing component was determined to be 0.097 N/cm (see Example 1). In this embodiment, to control the ODD, the weld spacing between the perimeter weld points of the lumen was less than 9 mm. With a similar stiffness (i.e., ˜0.097 N/cm) reinforcing component, it is possible to decrease the weld spacing to less than 9 mm to result in a decreased oxygen diffusion distance. Additionally, with an increased stiffness (i.e., greater than 0.097 N/cm) reinforcing component, it is possible to further reduce oxygen diffusion distances at the equivalent weld spacing (˜9 mm) or increase weld spacing (>9 mm) to maintain consistent oxygen diffusion distances.

In another embodiment, the oxygen diffusion distance (ODD) may be controlled through implantation technique and a mechanism to hold the cell encapsulation device in place in vivo, such as, for example, sutures to fix the cell encapsulation device to a desired location in the body or quilting to restrain the expansion of the lumen of the cell encapsulation device.

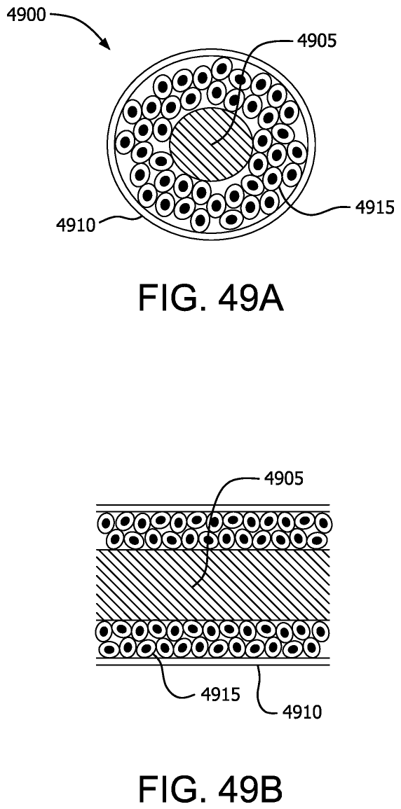

In some embodiments, the cell encapsulation device is structured such that the oxygen diffusion distance (ODD) is controlled by a cell displacing core. As shown in FIGS. 49A and 49B, the cell encapsulation device 4900 that includes a cell displacing core 4905 (e.g., spline) that is surrounded by a biocompatible membrane composite 4910. The space between the outer surface of the cell displacing core 4905 and the inner surface of the biocompatible membrane composite 4910 define a boundary zone in which cells 4915 may be contained. A maximum distance between the outer surface of the core 4905 which represents the hypothetical most interior cell and the inner surface of the permeable membrane 4910 (ODD) is sufficiently narrow to provide conditions suitable for the survival and function of the contained cells 4915, whereby the viability of a large proportion of the contained cells 4915 may be maintained. In particular, the cells 4915 contained within the cell encapsulation device 4900 are able to obtain nutrients and other biomolecules from the environment outside the cell encapsulation device 4900 and expel waste products and therapeutic substances outside the cell encapsulation device 4900 through the permeable membrane 4910.

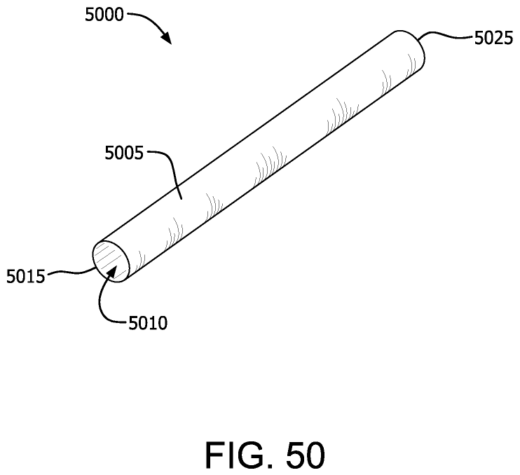

FIG. 50 shows the cell encapsulation device depicted in FIGS. 49A and 49B in a perspective view. The cell encapsulation device 5000 includes a first access port 5015, a second access port 5025, a biocompatible membrane composite 5005 forming the exterior of the encapsulation device 5000, and a lumen 5010 extending through the encapsulation device 5000. A cell displacing core (not illustrated) may be positioned within the lumen 5010 (and as shown in FIGS. 49A and 49B). In some embodiments, the cross-section of the cell encapsulation device 5000 may be circular, ovoid, or elliptical.

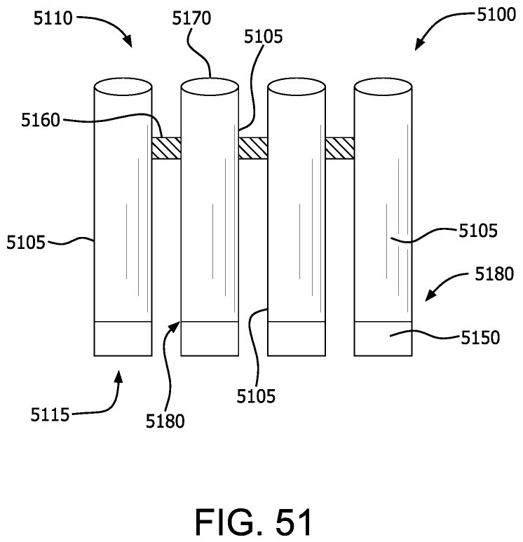

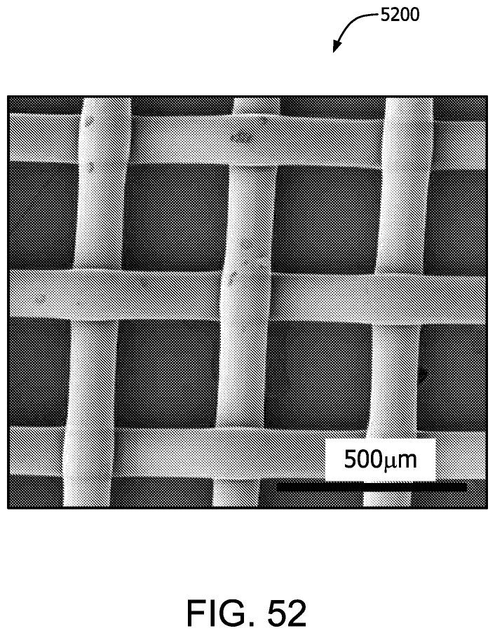

In some embodiments, the cell encapsulation device may contain multiple containment tubes. As shown in FIG. 51, the implantable device 5100 may include a plurality of interconnected cell encapsulation devices 5105 that are substantially parallel to each other along a length of the cell encapsulation device 5100. In the embodiment depicted in FIG. 51, the cell encapsulation devices 5105 are independently movable from each other, thus making the cell encapsulation device 5100 flexible and/or compliant with tissue and/or tissue movement. The cell encapsulation device 5105 may be configured to house a cell displacing core (not illustrated) along with cells. Each cell encapsulation device 5105 has a first access port 5170 at a proximal end 5110 and a second access port 5180 at a distal end 5115. The second access ports 5180 may have thereon resealable caps 5150 to seal the distal ends of the cell encapsulation devices 5105. Although not depicted, resealable caps may also be affixed to the first access ports 5170 to seal the proximal ends of the cell encapsulation device 5105. The cell encapsulation device 5105 may be interconnected at connection members 5160, for example, at their proximal ends. Similar tubular cell encapsulation devices are described in U.S. Patent Publication No. 2018/0126134 to Cully, et al.

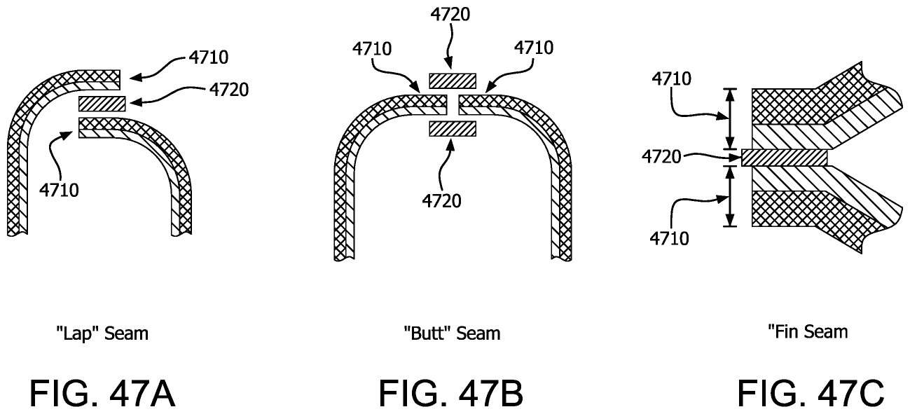

It is to be appreciated that the seams of the devices described herein, may alternatively or optionally be formed with one or more of a “lap” seam, a “butt” seam, or a “fin” seam as depicted in FIG. 47A-C, respectively. As shown in FIG. 47A, in a “lap” seam configuration, a thermoplastic weld film 4720 is sandwiched between two edges of a biocompatible membrane composite 4710. In the manufacture of an encapsulation device, a “lap” seam results from bonding the inner surface of one edge of a biocompatible membrane composite 4710 to the outer surface of the same or different biocompatible membrane composite 4710 (in the case of a single biocompatible membrane composite the resulting encapsulation device may have an edge with no seam (the same applies to FIGS. 47B-C). FIG. 47B shows a “butt” seam configuration where the sides of two ends of the same or different biocompatible membrane composite 4710 are in opposition to form a cell encapsulation device, while being sandwiched between two thermoplastic weld films 4720. FIG. 47C shows an exemplary “fin” seam configuration where the thermoplastic weld film 4720 is sandwiched between two edges of a biocompatible membrane composite 4710. The fin seam differs from the “lap” seam in that the two inner surfaces of the two edges of the biocompatible membrane composite 4710 are bonded through the thermoplastic weld film 4720. The resulting cell encapsulation device can be formed from one or a combination of seam configurations, such as, but not limited to, those depicted in FIGS. 47A-C. Additionally, there could be one or a plurality of different biocompatible membrane composites 4710 used in the construction of any of the cell encapsulation devices described herein.

Having generally described this disclosure, a further understanding can be obtained by reference to certain specific examples illustrated below which are provided for purposes of illustration only and are not intended to be all inclusive or limiting unless otherwise specified.

### Test Methods

### Porosity

The porosity of a layer is defined herein as the proportion of layer volume consisting of pore space compared to the total volume of the layer. The porosity is calculated by comparing the bulk density of a porous construct consisting of solid fraction and void fraction to the density of the solid fraction using the following equation:

$$Porosity = ( 1 - \frac{{Density}_{Bulk}}{{Density}_{SolidFraction}} ) \times 100 \% .$$

### Thickness

The thickness of the layers in the biocompatible membrane composites were measured by quantitative image analysis (QIA) of cross-sectional SEM images. Cross-sectional SEM images were generated by fixing membranes to an adhesive, cutting the film by hand using a liquid-nitrogen-cooled razor blade, and then standing the adhesive backed film on end such that the cross-section was vertical. The sample was then sputter coated using an Emitech K550X sputter coater (commercially available from Quorum Technologies Ltd, UK) and platinum target. The sample was then imaged using a FEI Quanta 400 scanning electron microscope from Thermo Scientific.

Layers within the cross-section SEM images were then measured for thickness using ImageJ 1.51 h from the National Institutes of Health (NIH). The image scale was set per the scale provided by the SEM. The layer of interest was isolated and cropped using the free-hand tool. A number of at least ten equally spaced lines were then drawn in the direction of the layer thickness. The lengths of all lines were measured and averaged to define the layer thickness.

### Stiffness

A stiffness test was performed based on ASTM D790-17 Standard test method for flexural properties of unreinforced and reinforced plastics and electrical insulating material. This method was used to determine the stiffness for biocompatible membrane composite layers and/or the final device.

Procedure B of the ASTM method was followed and includes greater than 5% strain and type 1 crosshead position for deflection. The dimensions of the fixture were adjusted to have a span of 16 mm and a radius of support and nosepiece of 1.6 mm. The test parameters used were a deflection of 3.14 mm and a test speed of 96.8 mm/min. In cases where the sample width differed from the standard 1 cm, the force was normalized to a 1 cm sample width by the linear ratio.

The load was reported in N/cm at maximum deflection.

### Tensile Strength

Materials were tested for tensile strength using a 5500 Series Instron® Electromechanical Testing System. Unless otherwise noted, materials were tested prior to the application of any coatings. Samples were cut using a D412F or D638-V dogbone die. The samples were then loaded into the Instron® tester grips and tested at a constant rate of 20 in/min (for D412F samples) or 3 in/min (for D683-V samples) until failure. Maximum load was normalized by test area (defined as gauge width times material thickness) to define tensile stress. Materials were tested in perpendicular directions (D1 and D2) and the maximum stress in each direction was used to calculate the geometric mean tensile strength of the material per the below equation:

$$Geometric Mean = \sqrt{{( Tensile {Strength}_{D 1} )}^{2} + {( Tensile {Strength}_{D 2} )}^{2}} .$$

### Maximum Tensile Load

Materials were tested for maximum tensile load using a 5500 Series Instron® Electromechanical Testing System. Samples were cut oriented in the axis of interest using a D412F or D638-V dogbone die. The samples were then loaded into the Instron® tester grips and tested at a constant rate of 20 in/min (for D412F samples) or 3 in/min (for D683-V samples) until failure. The maximum load sustained during testing was normalized by specimen gauge width (6.35 mm for D412F samples and 3.175 mm for D638-V samples) to define maximum tensile load.

### Composite Bond Strength (Z-Strength)

Materials were tested for composite bond strength using a 5500 Series Instron® Electromechanical Testing System. Unless otherwise noted, materials were tested prior to the application of any coatings. Samples were fixed to a 1″×1″ (2.54 cm×2.54 cm) steel platen using 3M 9500PC double sided tape and loaded into the Instron® with an opposing 1″×1″ steel platen with 3M 9500PC double sided tape on its surface. A characteristic compressive load of 1001 N was applied for 60 s to allow adhesive to partially penetrate the structure. After this bonding, the platens were separated at a constant rate of 20 in/s until failure. The maximum load was normalized by the test area (defined as the 1″×1″ test area) to define the composite bond.

### Mass/Area

Samples were cut (either by hand, laser, or die) to a known geometry. Unless otherwise noted, materials were tested prior to the application of any coatings. The dimensions of the sample were measured or verified and the area was calculated in m². The sample was then weighed in grams on a calibrated scale. The mass in grams was divided by the area in m² to calculate the mass per area in g/m².

### SEM Sample Preparation

SEM samples were prepared by first fixing the membrane composite or membrane composite layer(s)o an adhesive for handling, with the side opposite the side intended for imaging facing the adhesive. The film was then cut to provide an approximately 3 mm×3 mm area for imaging. The sample was then sputter coated using an Emitech K550X sputter coater and platinum target. Images were then taken using a FEI Quanta 400 scanning electron microscope from Thermo Scientific at a magnificent and resolution that allowed visualization of a sufficient number of features for robust analysis while ensuring each feature's minimum dimension was at least five pixels in length.

### Solid Feature Spacing

Solid feature was determined by analyzing SEM images in ImageJ 1.51 h from the National Institute of Health (NIH). The image scale was set based on the scale provided by the SEM image. Features were identified and isolated through a combination of thresholding based on size/shading and/or manual identification. In instances where the structure consists of a continuous structure, such as a nonwoven or etched surface, as opposed to a structure with discrete solid features, solid features are defined as the portion of the structure surrounding voids the their corresponding spacing extending from one side of the void to the opposing side. After isolating the features, a Delaunay Triangulation was performed to identify neighboring features. Triangulations whose circumcircle extended beyond the edge of the image were disregarded from the analysis. Lines were drawn between the nearest edges of neighboring features and measured for length to define spacing between neighboring features (see, e.g., FIG. 1A).

The median of all measured solid feature spacings marks the value that is less than or equal to half of the measured solid feature spacings and greater than or equal to half of the measured solid feature spacings. Therefore, if the measured median is above or below some value, the majority of measurements is similarly above or below the value. As such, the median is used as summary statistic to represent the majority of solid feature spacings.

### Measurement of Representative Minor Axis and Representative Major Axis

The representative minor axis was measured by analyzing SEM images of membrane surfaces in ImageJ 1.51 h from the NIH. The image scale was set based on the scale provided by the SEM image. Features were identified and isolated through a combination of thresholding based on size/shading and/or manual identification. After isolating the features, the built in particle analysis capabilities were leveraged to determine the major and minor axis of the representative ellipse. The minor axis of this ellipse is the representative minor axis of the measured feature. The major axis of this ellipse is the representative major axis of the measured feature. The median of all measured minor axes marks the value that is less than or equal to half of the measured minor axes and greater than or equal to half of the measured minor axes. Similarly, the median of all measured major axes marks the value that is less than or equal to half of the measured major axes and greater than or equal to half of the measured major axes. In both cases, if the measured median is above or below some value, the majority of measurements is similarly above or below the value. As such, the median is used as summary statistic to represent the majority of solid feature representative minor axes and representative major axes.

### Solid Feature Depth

Solid feature depth was determined by using quantitative image analysis (QIA) of SEM images of membrane cross-sections. Cross-sectional SEM images were generated by fixing films to an adhesive, cutting the film by hand using a liquid-nitrogen-cooled razor blade, and then standing the adhesive backed film on end such that the cross-section was vertical. The sample was then sputter coated using an Emitech K550X sputter coater (commercially available from Quorum Technologies Ltd, UK) and platinum target. The sample was then imaged using a FEI Quanta 400 scanning electron microscope from Thermo Scientific.

Features within the cross-section SEM images were then measured for depth using ImageJ 1.51 h from the National Institutes of Health (NIH). The image scale was set per the scale provided by the SEM. Features were identified and isolated through a combination of thresholding based on size/shading and/or manual identification. After isolating features, built in particle analysis capabilities were leveraged to calculate the Feret diameter and angle formed by the axis defined by the Feret diameter axis and horizontal plane for each solid feature. The Feret diameter is the furthest distance between any two points on a feature's boundary in the plane of the SEM image. The Feret diameter axis is the line defined by these two points. The projection of the Feret diameter of each solid feature in the direction of the layer thickness was calculated per the equation.

Projection_{Thickness}=sin θ*Length_{Longest Axis}.

The projection of the longest axis in the direction of the layer thickness is the solid feature depth of the measured feature. The median of all measured solid feature depths marks the value that is less than or equal to half of the measured solid feature depths and greater than or equal to half of the measured solid feature depths. Therefore, if the measured median is above or below some value, the majority of measurements is similarly above or below the value As such, the median is used as summary statistic to represent the majority of solid feature depths.

### Pore Size

The pore size was measured by analyzing SEM images of membrane surfaces in ImageJ 1.51 h from the NIH. The image scale was set based on the scale provided by the SEM image. Pores were identified and isolated through a combination of thresholding based on size/shading and/or manual identification. After isolating the pores, the built in particle analysis capabilities were leveraged to determine the area of each pore. The measured pore area was converted to an “effective diameter” per the below equation:

$$Effective Diameter = 2 \times \sqrt{\frac{Area}{π}}$$

The pore areas were summed to define the total area of the surface defined by pores. This is the total pore area of the surface. The pore size of a layer is the effective diameter of the pore that defines the point where roughly half the total pore area consists of pores with diameters smaller than the pore size and roughly half the total pore area consists of pores with diameters greater than or equal to the pore size.

### MPS (Maximum Pore Size)

MPS (maximum pore size) was measured per ASTM F316 using a Quantachrome 3 Gzh porometer from Anton Paar and silicone oil (20.1 dyne/cm) as a wetting solution.

### Oxygen Diffusion Distance (ODD)

In order to assess the oxygen diffusion distance (ODD) in vitro, a cell encapsulation device without cells therein is pressurized to 1.0 PSI to simulate an in vivo effect of the encapsulated cells. It is to be noted that the encapsulated cells are assumed to exert a pressure of approximately 1.0 PSI above the surrounding tissue.

To make a measurement of the ODD, the cell encapsulation device is first pressurized to a desired pressure (e.g., 1.0 PSI). Additionally, it is possible to perform this method at a range of different pressures (e.g. between 0.5 to 5 psi) and plot the change in ODD with internal pressure. The fluid used to pressurize the cell encapsulation device is not particularly limiting as long as the desired internal pressure can be accurately controlled. If there are additional layers (e.g., reinforcement components) on the external surface of the cell impermeable membrane which are expected to be penetrated by blood vessels in vivo, these layers are not included in the final measurement in order to accurately measure the ODD.

To calculate the ODD of a device that includes an open lumen that has no internal reinforcing component or additional layers or structures between opposing membrane composite layers the expansion of the lumen when pressurized is measured by assessing the change in thickness after internal pressurization. First, a measurement of the total device thickness is taken while the cell encapsulation device is in equilibrium pressure with the surrounding atmosphere. This measurement can be taken by any accurate thickness measurement method such as a non-contact gauge or a contact mechanical gauge as long as the measurement gauge does not appreciably change the recorded dimension. One non-limiting example of a measurement gauge that can be used is a drop gauge (Mitutoy, Absolute). An additional non-limiting example of a measurement technique that can be used is an optical measuring microscope or optical comparator (Keyence). Herein, this measurement is called the unpressurized dimension. Prior to measuring the unpressurized dimension, consideration should also be taken to any pre-conditioning of the cell encapsulation device. For example, the cell encapsulation device can undergo a simulated cell loading pre-conditioning step by pressurizing the device to a simulation pressure induced by cell loading (e.g. 5 psi) and then stepwise reducing the pressure down to a final pressure more consistent with the ODD method (e.g. 1 psi).

One method to pressurize the lumen is to wet the cell encapsulation device to render the cell impermeable membrane temporarily non-permeable to air. Isopropyl alcohol is one non-limiting example of a suitable wetting fluid. The encapsulation device is then pressurized, for example, with air at 1.0 PSI above the surrounding atmosphere with a pressure regulator. A second thickness measurement is taken while the cell encapsulation device is at the desired pressure at the same location used for the unpressurized dimension. Next, the unpressurized dimension is subtracted from the pressurized dimension to obtain the lumen expansion. The lumen expansion is then divided by two (2) to obtain the distance from the most interior portion of the lumen to the interior side of the cell impermeable layer (see, FIG. 12B). The maximum ODD is then calculated by adding the thickness of the cell impermeable membrane to the distance from the limiting cell location to the interior of the CIM. The maximum ODD is the point of greatest deflection of the device and is the largest ODD obtained anywhere on the cell encapsulation device. To calculate the majority ODD, multiple measurements (more than 5) need to be taken across the active surface area of the device. Care should be taken to ensure that a range of distances across the entire cross section of the device are assessed.

An alternate test method is needed where there is an internal reinforcing structure (e.g. a reinforcing component or structural pillars) within the lumen or positioned between opposing membrane composite layers. In this case the presence of the internal reinforcing structure limits the ability to get an accurate measurement in the unpressurized state because the thickness and location of the internal reinforcing structures would need to be assumed and it cannot be assured that any internal reinforcing structure equally divides the interior of the cell encapsulation device into two equal portions. To perform the alternate test method for cell encapsulation devices with an internal reinforcing structure, the lumen of the device is pressurized with a liquid that can be solidified, such as, for example, a 2 part silastic rubber (e.g., Reprorubber thin pour model 16301 available from Flexbar Machine Corporation, Islandia, N.Y.) or a 2 part epoxy (e.g. Master Bond EP30LV from Master Bond Inc., Hackensack, N.J.). A final ODD measurement can be taken directly after cross-sectioning the cell encapsulation device, provided any change in dimension upon the liquid solidification is taken into account. The ODD can be measured using a solidified cross section at the point of maximum deflection to determine the maximum ODD. Additionally, the ODD can be measured using solidified cross sections at multiple locations throughout the active surface area of the device by taking multiple measurements (more than 5) across the entire width of the cross section to determine the majority ODD.

### In Vitro Production of Human PDX1-Positive Pancreatic Endoderm and Endocrine Cells

The directed differentiation methods herein for pluripotent stem cells, for example, hES and iPS cells, can be described into at least four or five or six or seven stages, depending on end-stage cell culture or cell population desired (e.g. PDX1-positive pancreatic endoderm cell population (or PEC), or endocrine precursor cell population, or endocrine cell population, or immature beta cell population or mature endocrine cell population).

Stage 1 is the production of definitive endoderm from pluripotent stem cells and takes about 2 to 5 days, preferably 2 or 3 days. Pluripotent stem cells are suspended in media comprising RPMI, a TGFβ superfamily member growth factor, such as Activin A, Activin B, GDF-8 or GDF-11 (100 ng/mL), a Wnt family member or Wnt pathway activator, such as Wnt3a (25 ng/mL), and alternatively a rho-kinase or ROCK inhibitor, such as Y-27632 (10 μM) to enhance growth, and/or survival and/or proliferation, and/or cell-cell adhesion. After about 24 hours, the media is exchanged for media comprising RPMI with serum, such as 0.2% FBS, and a TGFβ superfamily member growth factor, such as Activin A, Activin B, GDF-8 or GDF-11 (100 ng/mL), and alternatively a rho-kinase or ROCK inhibitor for another 24 (day 1) to 48 hours (day 2). Alternatively, after about 24 hours in a medium comprising Activin/Wnt3a, the cells are cultured during the subsequent 24 hours in a medium comprising Activin alone (i.e., the medium does not include Wnt3a). Importantly, production of definitive endoderm requires cell culture conditions low in serum content and thereby low in insulin or insulin-like growth factor content. See McLean et al. (2007) Stem Cells 25: 29-38. McLean et al. also show that contacting hES cells with insulin in concentrations as little as 0.2 μg/mL at Stage 1 can be detrimental to the production of definitive endoderm. Still others skilled in the art have modified the Stage 1 differentiation of pluripotent cells to definitive endoderm substantially as described here and in D'Amour et al. (2005), for example, at least, Agarwal et al., Efficient Differentiation of Functional Hepatocytes from Human Embryonic Stem Cells, Stem Cells (2008) 26:1117-1127; Borowiak et al., Small Molecules Efficiently Direct Endodermal Differentiation of Mouse and Human Embryonic Stem Cells, (2009) Cell Stem Cell 4:348-358; Brunner et al., Distinct DNA methylation patterns characterize differentiated human embryonic stem cells and developing human fetal liver, (2009) Genome Res. 19:1044-1056, Rezania et al. Reversal of Diabetes with Insulin-producing Cells Derived In Vitro from Human Pluripotent Stem Cells (2014) Nat Biotech 32(11): 1121-1133 (GDF8 & GSK3beta inhibitor, e.g. CHIR99021); and Pagliuca et al. (2014) Generation of Function Human Pancreatic B-cell In Vitro, Cell 159: 428-439 (Activin A & CHIR) Proper differentiation, specification, characterization and identification of definitive are necessary in order to derive other endoderm-lineage cells. Definitive endoderm cells at this stage co-express SOX17 and HNF3β (FOXA2) and do not appreciably express at least HNF4alpha, HNF6, PDX1, SOX6, PROX1, PTF1A, CPA, cMYC, NKX6.1, NGN3, PAX3, ARX, NKX2.2, INS, GSC, GHRL, SST, or PP. The absence of HNF4alpha expression in definitive endoderm is supported and described in detail in at least Duncan et al. (1994), Expression of transcription factor HNF-4 in the extraembryonic endoderm, gut, and nephrogenic tissue of the developing mouse embryo: HNF-4 is a marker for primary endoderm in the implanting blastocyst,” Proc. Natl. Acad. Sci, 91:7598-7602 and Si-Tayeb et al. (2010), Highly Efficient Generation of Human Hepatocyte-Like cells from Induced Pluripotent Stem Cells,” Hepatology 51:297-305.

Stage 2 takes the definitive endoderm cell culture from Stage 1 and produces foregut endoderm or PDX1-negative foregut endoderm by incubating the suspension cultures with RPMI with low serum levels, such as 0.2% FBS, in a 1:1000 dilution of ITS, 25 ng KGF (or FGF7), and alternatively a ROCK inhibitor for 24 hours (day 2 to day 3). After 24 hours (day 3 to day 4), the media is exchanged for the same media minus a TGFβ inhibitor, but alternatively still a ROCK inhibitor to enhance growth, survival and proliferation of the cells, for another 24 (day 4 to day 5) to 48 hours (day 6). A critical step for proper specification of foregut endoderm is removal of TGFβ family growth factors. Hence, a TGFβ inhibitor can be added to Stage 2 cell cultures, such as 2.5 μM TGFβ inhibitor no. 4 or 5 μM SB431542, a specific inhibitor of activin receptor-like kinase (ALK), which is a TGFβ type I receptor. Foregut endoderm or PDX1-negative foregut endoderm cells produced from Stage 2 co-express SOX17, HNF1p and HNF4alpha and do not appreciably co-express at leasHNF3β (FOXA2), nor HNF6, PDX1, SOX6, PROX1, PTF1A, CPA, cMYC, NKX6.1, NGN3, PAX3, ARX, NKX2.2, INS, GSC, GHRL, SST, or PP, which are hallmark of definitive endoderm, PDX1-positive pancreatic endoderm or pancreatic progenitor cells or endocrine progenitor/precursors as well as typically poly hormonal type cells.

Stage 3 (days 5-8) for PEC production takes the foregut endoderm cell culture from Stage 2 and produces a PDX1-positive foregut endoderm cell by DMEM or RPMI in 1% B27, 0.25 μM KAAD cyclopamine, a retinoid, such as 0.2 retinoic acid (RA) or a retinoic acid analog such as 3 nM of TTNPB (or CTT3, which is the combination of KAAD cyclopamine and TTNPB), and 50 ng/mL of Noggin for about 24 (day 7) to 48 hours (day 8). Specifically, Applicants have used DMEM-high glucose since about 2003 and all patent and non-patent disclosures as of that time employed DMEM-high glucose, even if not mentioned as “DMEM-high glucose” and the like. This is, in part, because manufacturers such as Gibco did not name their DMEM as such, e.g. DMEM (Cat. No 11960) and Knockout DMEM (Cat. No 10829). It is noteworthy, that as of the filing date of this application, Gibco offers more DMEM products but still does not put “high glucose” in certain of their DMEM products that contain high glucose e.g. Knockout DMEM (Cat. No. 10829-018). Thus, it can be assumed that in each instance DMEM is described, it is meant DMEM with high glucose and this was apparent by others doing research and development in this field. Again, a ROCK inhibitor or rho-kinase inhibitor such as Y-27632 can be used to enhance growth, survival, proliferation and promote cell-cell adhesion. Additional agents and factors include but are not limited to ascorbic acid (e.g. Vitamin C), BMP inhibitor (e.g. Noggin, LDN, Chordin), SHH inhibitor (e.g. SANT, cyclopamine, HIP1); and/or PKC activator (e.g. PdBu, TBP, ILV) or any combination thereof. Alternatively, Stage 3 has been performed without an SHH inhibitor such as cyclopamine in Stage 3. PDX1-positive foregut cells produced from Stage 3 co-express PDX1 and HNF6 as well as SOX9 and PROX, and do not appreciably co-express markers indicative of definitive endoderm or foregut endoderm (PDX1-negative foregut endoderm) cells or PDX1-positive foregut endoderm cells as described above in Stages 1 and 2.

The above stage 3 method is one of four stages for the production of PEC populations. For the production of endocrine progenitor/precursor and endocrine cells as described in detail below, in addition to Noggin, KAAD-cyclopamine and Retinoid; Activin, Wnt and Heregulin, thyroid hormone, TGFb-receptor inhibitors, Protein kinase C activators, Vitamin C, and ROCK inhibitors, alone and/or combined, are used to suppress the early expression NGN3 and increasing CHGA-negative type of cells.

Stage 4 (about days 8-14) PEC culture production takes the media from Stage 3 and exchanges it for media containing DMEM in 1% vol/vol B27 supplement, plus 50 ng/mL KGF and 50 ng/mL of EGF and sometimes also 50 ng/mL Noggin and a ROCK inhibitor and further includes Activin alone or combined with Heregulin. Alternatively, Stage 3 cells can be further differentiated using KGF, RA, SANT, PKC activator and/or Vitamin C or any combination thereof. These methods give rise to pancreatic progenitor cells co-expressing at least PDX1 and NKX6.1 as well as PTF1A. These cells do not appreciably express markers indicative of definitive endoderm or foregut endoderm (PDX1-negative foregut endoderm) cells as described above in Stages 1, 2 and 3.

Stage 5 production takes Stage 4 PEC cell populations above and further differentiates them to produce endocrine progenitor/precursor or progenitor type cells and/or singly and poly-hormonal pancreatic endocrine type cells in a medium containing DMEM with 1% vol/vol B27 supplement, Noggin, KGF, EGF, RO (a gamma secretase inhibitor), nicotinamide and/or ALK5 inhibitor, or any combination thereof, e.g. Noggin and ALK5 inhibitor, for about 1 to 6 days (preferably about 2 days, i.e. days 13-15). Alternatively, Stage 4 cells can be further differentiated using retinoic acid (e.g. RA or an analog thereof), thyroid hormone (e.g. T3, T4 or an analogue thereof), TGFb receptor inhibitor (ALK5 inhibitor), BMP inhibitor (e.g. Noggin, Chordin, LDN), or gamma secretase inhibitor (e.g., XXI, XX, DAPT, XVI, L685458), and/or betacellulin, or any combination thereof. Endocrine progenitor/precursors produced from Stage 5 co-express at least PDX1/NKX6.1 and also express CHGA, NGN3 and Nkx2.2, and do not appreciably express markers indicative of definitive endoderm or foregut endoderm (PDX1-negative foregut endoderm) as described above in Stages 1, 2, 3 and 4 for PEC production.

Stage 6 and 7 can be further differentiated from Stage 5 cell populations by adding any of a combination of agents or factors including but not limited to PDGF+SSH inhibitor (e.g. SANT, cyclopamine, HIP1), BMP inhibitor (e.g. Noggin, Chordin, LDN), nicotinamide, insulin-like growth factor (e.g. IGF1, IGF2), TTNBP, ROCK inhibitor (e.g. Y27632), TGFb receptor inhibitor (e.g. ALK5i), thyroid hormone (e.g. T3, T4 and analogues thereof), and/or a gamma secretase inhibitor (XXI, XX, DAPT, XVI, L685458) or any combination thereof to achieve the cell culture populations or appropriate ratios of endocrine cells, endocrine precursors and immature beta cells.

Stage 7 or immature beta cells are considered endocrine cells but may or may not me sufficiently mature to respond to glucose in a physiological manner. Stage 7 immature beta cells may express MAFB, whereas MAFA and MAFB expressing cells are fully mature cells capable of responding to glucose in a physiological manner.

Stages 1 through 7 cell populations are derived from human pluripotent stem cells (e.g. human embryonic stem cells, induced pluripotent stem cells, genetically modified stem cells e.g. using any of the gene editing tools and applications now available or later developed) and may not have their exact naturally occurring corresponding cell types since they were derived from immortal human pluripotent stem cells generated in vitro (i.e. in an artificial tissue culture) and not the inner cell mass in vivo (i.e. in vivo human development does not have an human ES cell equivalent).

Pancreatic cell therapy replacements as intended herein can be encapsulated in the described herein devices consisting of herein described membranes using any of Stages 4, 5, 6 or 7 cell populations and are loaded and wholly contained in a macro-encapsulation device and transplanted in a patient, and the pancreatic endoderm lineage cells mature into pancreatic hormone secreting cells, or pancreatic islets, e.g., insulin secreting beta cells, in vivo (also referred to as “in vivo function”) and are capable of responding to blood glucose normally.

Encapsulation of the pancreatic endoderm lineage cells and production of insulin in vivo is described in detail in U.S. application Ser. No. 12/618,659 (the '659 application), entitled ENCAPSULATION OF PANCREATIC LINEAGE CELLS DERIVED FROM HUMAN PLURIPOTENT STEM CELLS, filed Nov. 13, 2009. The '659 application claims the benefit of priority to Provisional Patent Application No. 61/114,857, entitled ENCAPSULATION OF PANCREATIC PROGENITORS DERIVED FROM HES CELLS, filed Nov. 14, 2008; and U.S. Provisional Patent Application No. 61/121,084, entitled ENCAPSULATION OF PANCREATIC ENDODERM CELLS, filed Dec. 9, 2008; and now U.S. Pat. Nos. 8,278,106 and 8,424,928. The methods, compositions and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. Accordingly, it will be apparent to one skilled in the art that varying substitutions and modifications may be made to the invention disclosed herein without departing from the scope and spirit of the invention.

Additionally, embodiments described herein are not limited to any one type of pluripotent stem cell or human pluripotent stem cell and include but are not limited to human embryonic stem (hES) cells and human induced pluripotent stem (iPS) cells or other pluripotent stem cells later developed. It is also well known in the art, that as of the filing of this application, methods for making human pluripotent stems may be performed without destruction of a human embryo and that such methods are anticipated for production of any human pluripotent stem cell.

Methods for producing pancreatic cell lineages from human pluripotent cells were conducted substantially as described in at least the listed publications assigned to ViaCyte, Inc. including but not limited to: PCT/US2007/62755 (WO2007101130), PCT/US2008/80516 (WO2009052505), PCT/US2008/82356 (WO2010053472), PCT/US2005/28829 (WO2006020919), PCT/US2014/34425 (WO2015160348), PCT/US2014/60306 (WO2016080943), PCT/US2016/61442 (WO2018089011), PCT/US2014/15156 (WO2014124172), PCT/US2014/22109 (WO2014138691), PCT/US2014/22065 (WO2014138671), PCT/US2005/14239 (WO2005116073), PCT/US2004/43696 (WO2005063971), PCT/US2005/24161 (WO2006017134), PCT/US2006/42413 (WO2007051038), PCT/US2007/15536 (WO2008013664), PCT/US2007/05541 (WO2007103282), PCT/US2008/61053 (WO2009131568), PCT/US2008/65686 (WO2009154606), PCT/US2014/15156 (WO2014124172), PCT/US2018/41648 (WO2019014351), PCT/US2014/26529 (WO2014160413), PCT/US2009/64459 (WO2010057039); and d'Amour et al. 2005 Nature Biotechnology 23:1534-41; D'Amour et al. 2006 Nature Biotechnology 24(11):1392-401; McLean et al., 2007 Stem Cells 25:29-38, Kroon et al. 2008 Nature Biotechnology 26(4): 443-452, Kelly et al. 2011 Nature Biotechnology 29(8): 750-756, Schulz et al., 2012 PLos One 7(5):e37004; and/or Agulnick et al. 2015 Stem Cells Transl. Med. 4(10):1214-22.

Methods for producing pancreatic cell lineages from human pluripotent cells were conducted substantially as described in at least the listed below publications assigned to Janssen including but not limited to: PCT/US2008/68782 (WO200906399), PCT/US2008/71775 (WO200948675), PCT/US2008/71782 (WO200918453), PCT/US2008/84705 (WO200970592), PCT/US2009/41348 (WO2009132063), PCT/US2009/41356 (WO2009132068), PCT/US2009/49183 (WO2010002846), PCT/US2009/61635 (WO2010051213), PCT/US2009/61774 (WO2010051223), PCT/US2010/42390 (WO2011011300), PCT/US2010/42504 (WO2011011349), PCT/US2010/42393 (WO2011011302), PCT/US2010/60756 (WO2011079017), PCT/US2011/26443 (WO2011109279), PCT/US2011/36043 (WO2011143299), PCT/US2011/48127 (WO2012030538), PCT/US2011/48129 (WO2012030539), PCT/US2011/48131 (WO2012030540), PCT/US2011/47410 (WO2012021698), PCT/US2012/68439 (WO2013095953), PCT/US2013/29360 (WO2013134378), PCT/US2013/39940 (WO2013169769), PCT/US2013/44472 (WO2013184888), PCT/US2013/78191 (WO2014106141), PCTU/S2014/38993 (WO2015065524), PCT/US2013/75939 (WO2014105543), PCT/US2013/75959 (WO2014105546), PCT/US2015/29636 (WO2015175307), PCT/US2015/64713 (WO2016100035), PCT/US2014/41988 (WO2015002724), PCT/US2017/25847 (WO2017180361), PCT/US2017/37373 (WO2017222879), PCT/US2017/37373 (WO2017222879); PCT/US2009/049049 (WO2010/002785), PCT/US2010/060770 (WO2011/079018), PCT/US2014/042796, (WO2015/065537), PCT/US2008/070418 (WO2009/012428); Bruin et al. 2013 Diabetologia. 56(9): 1987-98, Fryer et al. 2013 Curr. Opin. Endocrinol. Diabetes Obes. 20(2): 112-7, Chetty et al. 2013 Nature Methods. 10(6):553-6, Rezania et al. 2014 Nature Biotechnologyy 32(11):1121-33, Bruin et al. 2014 Stem Cell Res. 12(1): 194-208, Hrvatin 2014 Proc. Natl. Acad. Sci. USA. 111(8): 3038-43, Bruin et al. 2015 Stem Cell Reports. 5, 1081-1096, Bruin et al. 2015 Science Transl. Med., 2015, 7, 316ps23, and/or Bruin et al. 2015 Stem Cell Reports. 14; 4(4):605-20.

In one embodiment, human pluripotent cells were differentiated to PDX1-positive pancreatic endodermcells including pancreatic progenitors and endocrine precursors according to one of the preferred following conditions A and/or B.

TABLE 1
Media Conditions for PDX1-positive Pancreatic
Endoderm Cell Production

| Stage | A | B |
|---|---|---|
| 1 | r0.2FBS-ITS1:5000 A100 W50 | |
| | r0.2FBS-ITS1:5000 A100 | |
| 2 | r0.2FBS-ITS1:1000 K25 IV | |
| | r0.2FBS-ITS1:1000 K25 | |
| | r0.2FBS-ITS1:1000 K25 | |
| 3 | db-TT3 N50 | |
| | db-TT3 N50 | |
| | db-TT3 N50 | |
| 4 | db-N50 K50 E50 | |
| | db-N50 K50 E50 | |
| | db-N50 K50 E50 | |
| | db-N50 K50 E50 → Cryopreserved | |
| Thaw | db-N50 K50 E50 | db-N100 A5i (1uM) |
| (S5- | db-N50 K50 E50 | db-N100 A5i (1uM) |
| S6) | db-N50 K50 E50 | db-N100 A5i (1uM) |
| | | db-N100 A5i (10uM) |
| | | db-A5i (10uM) |
| | | db-A5i (10uM) |

Table 1 Legend: r0.2FBS: RPMI 1640 (Mediatech); 0.2% FBS (HyClone), 1x GlutaMAX-1 (Life Technologies), 1% v/v penicillin/streptomycin; db: DMEM Hi Glucose (HyClone) supplemented with 0.5x B-27 Supplement (Life Technologies); A100, A50, AS: 100 ng/mL recombinant human Activin A (R&D Systems); A5i: 1uM, 5uM, 10uM ALK5 inhibitor; TT3: 3 nM TTNPB (Sigma-Aldrich); E50: 50 ng/mL recombinant human EGF (R&D Systems); ITS: Insulin-Transferrin-Selenium (Life Technologies) diluted 1:5000 or 1:1000; IV: 2.5 mM TGF-b RI Kinase inhibitor IV (EMD Bioscience); K50, K25: 50 ng/mL, 25 ng/mL recombinant human KGF (R&D Systems, or Peprotech); N50, N100: 50 ng/mL or 100 ng/mL recombinant human Noggin (R&D Systems); W50: 50 ng/mL recombinant mouse Wnt3A (R&D Systems).

One of ordinary skill in the art will appreciate that there may exist other methods for production of PDX1-positive pancreatic endoderm cells or PDX1-positive pancreatic endoderm lineage cells including pancreatic progenitors or even endocrine and endocrine precursor cells; and at least those PDX1-positive pancreatic endoderm cells described in Kroon et al. 2008, Rezania et al. 2014 supra and Pagliuca et al. 2014 Cell 159(2):428-439, supra.

One of ordinary skill in the art will also appreciate that the embodiments described herein for production of PDX1-positive pancreatic endoderm cells consists of a mixed population or a mixture of subpopulations. And because unlike mammalian in vivo development which occurs along the anterior-posterior axis, and cells and tissues are named such accordingly, cell cultures in any culture vessels lack such directional patterning and thus have been characterized in particular due to their marker expression. Hence, mixed subpopulations of cells at any stage of differentiation does not occur in vivo. The PDX1-positive pancreatic endoderm cell cultures therefore include, but are not limited to: i) endocrine precursors (as indicated e.g. by the early endocrine marker, Chromogranin A or CHGA); ii) singly hormonal polyhormonal cells expressing any of the typical pancreatic hormones such as insulin (INS), somatostatin (SST), pancreatic polypeptide (PP), glucagon (GCG), or even gastrin, incretin, secretin, or cholecystokinin; iii) pre-pancreatic cells, e.g. cells that express PDX-1 but not NKX6.1 or CHGA; iv) endocrine cells that co-express PDX-1/NKX6.1 and CHGA (PDX-1/NKX6.1/CHGA), or non-endocrine e.g., PDX-1/NKX6.1 but not CHGA (PDX-1+/NKX6.1+/CHA−); and v) still there are cells that do not express PDX-1, NKX6.1 or CHGA (e.g. triple negative cells).

This PDX1-positive pancreatic endoderm cells population with its mixed subpopulations of cells mostly express at least PDX-1, in particular a subpopulation that expresses PDX-1/NKX6.1. The PDX1/NKX6.1 subpopulation has also been referred to as “pancreatic progenitors”, “Pancreatic Epithelium” or “PEC” or versions of PEC, e.g. PEC-01. Although Table 1 describes a stage 4 population of cells, these various subpopulations are not limited to just stage 4. Certain of these subpopulations can be for example found in as early as stage 3 and in later stages including stages 5, 6 and 7 (immature beta cells). The ratio of each subpopulation will vary depending on the cell culture media conditions employed. For example, in Agulnick et al. 2015, supra, 73-80% of PDX-1/NKX6.1 cells were used to further differentiate to islet-like cells (ICs) that contained 74-89% endocrine cells generally, and 40-50% of those expressed insulin (INS). Hence, different cell culture conditions are capable of generating different ratios of subpopulations of cells and such may effect in vivo function and therefore blood serum c-peptide levels. And whether modifying methods for making PDX1-positive pancreatic endoderm lineage cell culture populations effects in vivo function can only be determined using in vivo studies as described in more detail below. Further, it cannot be assumed and should not be assumed that just because a certain cell type has been made and has well characterized, that such a method produces the same cell intermediates, unless this is also well characterized.

In one aspect, a method for producing mature beta cells in vivo is provided. The method consisting of making human definitive endoderm lineage cells derived from human pluripotent stem cells in vitro with at least a TGFβ superfamily member and/or at least a TGFβ superfamily member and a Wnt family member, preferably a TGFβ superfamily member and a Wnt family member, preferably Activin A, B or GDF-8, GDF-11 or GDF-15 and Wnt3a, preferably Actvin A and Wnt3a, preferably GDF-8 and Wnt3a. The method for making PDX1-positive pancreatic endoderm cells from definitive endoderm cells with at least KGF, a BMP inhibitor and a retinoic acid (RA) or RA analog, and preferably with KGF, Noggin and RA. The method may further differentiate the PDX1-positive pancreatic endoderm cells into immature beta cells or MAFA expressing cells with a thyroid hormone and/or a TGFb-RI inhibitor, a BMP inhibitor, KGF, EGF, a thyroid hormone, and/or a Protein Kinase C activator; preferably with noggin, KGF and EGF, preferably additionally with T3 or T4 and ALK5 inhibitor or T3 or T4 alone or ALK5 inhibitor alone, or T3 or T4, ALK5 inhibitor and a PKC activator such as ILV, TPB and PdBu. Or preferably with noggin and ALK5i and implanting and maturing the PDX1-positive pancreatic endoderm cells or the MAFA immature beta cell populations into a mammalian host in vivo to produce a population of cells including insulin secreting cells capable of responding to blood glucose.

In one aspect, a unipotent human immature beta cell or PDX1-positive pancreatic endoderm cell that expresses INS and NKX6.1 and does not substantially express NGN3 is provided. In one embodiment, the unipotent human immature beta cell is capable of maturing to a mature beta cell. In one embodiment, the unipotent human immature beta cell further expresses MAFB in vitro and in vivo. In one embodiment, the immature beta cells express INS, NKX6.1 and MAFA and do not substantially express NGN3.

In one aspect, pancreatic endoderm lineage cells expressing at least CHGA (or CHGA+) refer to endocrine cells; and pancreatic endoderm cells that do not express CHGA (or CHGA−) refer to non-endocrine cells. In another aspect, these endocrine and non-endocrine sub-populations may be multipotent progenitor/precursor sub-populations such as non-endocrine multipotent pancreatic progenitor sub-populations or endocrine multipotent pancreatic progenitor sub-populations; or they may be unipotent sub-populations such as immature endocrine cells, preferably immature beta cells, immature glucagon cells and the like.

In one aspect, more than 10% preferably more than 20%, 30%, 40% and more preferably more than 50%, 60%, 70%, 80%, 90%, 95%, 98% or 100% of the cells in the pancreatic endoderm or PDX1-positive pancreatic endoderm cell population (stage 4) are the non-endocrine (CHGA−) multipotent progenitor sub-population that give rise to mature insulin secreting cells and respond to glucose in vivo when implanted into a mammalian host.

One embodiment provides a composition and method for differentiating pluripotent stem cells in vitro to substantially pancreatic endoderm cultures and further differentiating the pancreatic endoderm culture to endocrine or endocrine precursor cells in vitro. In one aspect, the endocrine precursor or endocrine cells express CHGA. In one aspect, the endocrine cells can produce insulin in vitro. In one aspect, the in vitro endocrine insulin secreting cells may produce insulin in response to glucose stimulation. In one aspect, more than 10% preferably more than 20%, 30%, 40% and more preferably more than 50%, 60%, 70%, 80%, 90%, 95%, 98% A or 100% of the cells in the cells population are endocrine cells.

Embodiments described herein provide for compositions and methods of differentiating pluripotent human stem cells in vitro to endocrine cells. In one aspect, the endocrine cells express CHGA. In one aspect, the endocrine cells can produce insulin in vitro. In one aspect, the endocrine cells are immature endocrine cells such as immature beta cells. In one aspect, the in vitro insulin producing cells may produce insulin in response to glucose stimulation.

One embodiment provides a method for producing insulin in vivo in a mammal, the method comprising: (a) loading a pancreatic endoderm cell or endocrine cell or endocrine precursor cell population into an implantable semi-permeable device; (b) implanting the device with the cell population into a mammalian host; and (c) maturing the cell population in the device in vivo wherein at least some of the endocrine cells are insulin secreting cells that produce insulin in response to glucose stimulation in vivo, thereby producing insulin in vivo to the mammal. In one aspect the endocrine cell is derived from a cell composition comprising PEC with a higher non-endocrine multipotent pancreatic progenitor sub-population (CHGA−). In another aspect, the endocrine cell is derived from a cell composition comprising PEC with a reduced endocrine sub-population (CHGA+). In another aspect, the endocrine cell is an immature endocrine cell, preferably an immature beta cell.

In one aspect the endocrine cells made in vitro from pluripotent stem cells express more PDX1 and NKX6.1 as compared to PDX-1 positive pancreatic endoderm populations, or the non-endocrine (CHGA−) subpopulations which are PDX1/NKX6.1 positive. In one aspect, the endocrine cells made in vitro from pluripotent stem cells express PDX1 and NKX6.1 relatively more than the PEC non-endocrine multipotent pancreatic progenitor sub-population (CHGA−). In one aspect, a Bone Morphogenic Protein (BMP) and a retinoic acid (RA) analog alone or in combination are added to the cell culture to obtain endocrine cells with increased expression of PDX1 and NKX6.1 as compared to the PEC non-endocrine multipotent progenitor sub-population (CHGA−). In one aspect BMP is selected from the group comprising BMP2, BMP5, BMP6, BMP7, BMP8 and BMP4 and more preferably BMP4. In one aspect the retinoic acid analog is selected from the group comprising all-trans retinoic acid and TTNPB (4-[(E)-2-(5,6,7,8-Tetrahydro-5,5,8,8-tetramethyl-2-naphthalenyl)-1-propenyl]benzoic acid Arotinoid acid), or 0.1-10 μM AM-580 (4-[(5,6,7,8-Tetrahydro-5,5,8,8-tetramethyl-2-naphthalenyl)carboxamido]benzoic acid) and more preferably TTNPB.

One embodiment provides a method for differentiating pluripotent stem cells in vitro to endocrine and immature endocrine cells, preferably immature beta cells, comprising dissociating and re-associating the aggregates. In one aspect the dissociation and re-association occurs at stage 1, stage 2, stage 3, stage 4, stage 5, stage 6 or stage 7 or combinations thereof. In one aspect the definitive endoderm, PDX1-negative foregut endoderm, PDX1-positive foregut endoderm, PEC, and/or endocrine and endocrine progenitor/precursor cells are dissociated and re-associated. In one aspect, the stage 7 dissociated and re-aggregated cell aggregates consist of fewer non-endocrine (CHGA−) sub-populations as compared to endocrine (CHGA+) sub-populations. In one aspect, more than 10% preferably more than 20%, 30%, 40% and more preferably more than 50%, 60%, 70%, 80%, 90%, 95%, 98% or 100% of the cells in the cell population are endocrine (CHGA+) cells.

One embodiment provides a method for differentiating pluripotent stem cells in vitro to endocrine cells by removing the endocrine cells made during stage 4 PEC production thereby enriching for non-endocrine multipotent pancreatic progenitor (CHGA−) sub-population which is PDX1+ and NKX6.1+.

In one embodiment, PEC cultures enriched for the non-endocrine multipotent progenitor sub-population (CHGA−) are made by not adding a Noggin family member at stage 3 and/or stage 4. In one embodiment, PEC cultures which are relatively replete of cells committed to the endocrine lineage (CHGA+) are made by not adding a Noggin family member at stage 3 and/or stage 4. In one aspect the Noggin family member is a compound selected from the group comprising Noggin, Chordin, Follistatin, Folistatin-like proteins, Cerberus, Coco, Dan, Gremlin, Sclerostin, PRDC (protein related to Dan and Cerberus).

One embodiment provides a method for maintaining endocrine cells in culture by culturing them in a media comprising exogenous high levels of glucose, wherein the exogenous glucose added is about 1 mM to 25 mM, about 1 mM to 20 mM, about 5 mM to 15 mM, about 5 mM to 10 mM, about 5 mM to 8 mM. In one aspect, the media is a DMEM, CMRL or RPMI based media.

One embodiment provides a method for differentiating pluripotent stem cells in vitro to endocrine cells with and without dissociating and re-associating the cell aggregates. In one aspect the non-dissociated or the dissociated and re-associated cell aggregates are cryopreserved or frozen at stage 6 and/or stage 7 without affecting the in vivo function of the endocrine cells. In one aspect, the cryopreserved endocrine cell cultures are thawed, cultured and, when transplanted, function in vivo.

Another embodiment provides a culture system for differentiating pluripotent stem cells to endocrine cells, the culture system comprising of at least an agent capable of suppressing or inhibiting endocrine gene expression during early stages of differentiation and an agent capable of inducing endocrine gene expression during later stages of differentiation. In one aspect, an agent capable of suppressing or inhibiting endocrine gene expression is added to the culture system consisting of pancreatic PDX1 negative foregut cells. In one aspect, an agent capable of inducing endocrine gene expression is added to the culture system consisting of PDX1-positive pancreatic endoderm progenitors or PEC. In one aspect, an agent capable of suppressing or inhibiting endocrine gene expression is an agent that activates a TGFbeta receptor family, preferably it is Activin, preferably, it is high levels of Activin, followed by low levels of Activin. In one aspect, an agent capable of inducing endocrine gene expression is a gamma secretase inhibitor selected from a group consisting of N—[N-(3,5-Diflurophenacetyl-L-alanyl)]-S-phenylglycine t-Butyl Ester (DAPT), R044929097, DAPT (N—[N-(3,5-Difluorophenacetyl-L-alanyl)]-S-phenylglycine t-Butyl Ester), 1-(S)-endo-N-(1,3,3)-Trimethylbicyclo[2.2.1]hept-2-yl)-4-fluorophenyl Sulfonamide, WPE-III31C, S-3-[N′-(3,5-difluorophenyl-alpha-hydroxyacetyl)-L-alanilyl]amino-2,3-dih-ydro-1-methyl-5-phenyl-1H-1,4-benzodiazepin-2-one, (N)—[(S)-2-hydroxy-3-methyl-butyryl]-1-(L-alaninyl)-(S)-1-amino-3-methyl-4,5,6,7-tetrahydro-2H-3-benzazepin-2-one, BMS-708163 (Avagacestat), BMS-708163, Semagacestat (LY450139), Semagacestat (LY450139), MK-0752, MK-0752, YO-01027, YO-01027 (Dibenzazepine, DBZ), LY-411575, LY-411575, or LY2811376. In one aspect, high levels of Activin is meant levels greater than 40 ng/mL, 50 ng/mL, and 75 ng/mL. In one aspect, high levels of Activin are used during stage 3 or prior to production of pancreatic foregut endoderm cells. In one aspect, low levels of Activin means less than 30 ng/mL, 20 ng/mL, 10 ng/mL and 5 ng/mL. In one aspect, low levels of Activin are used during stage 4 or for production of PEC. In one aspect, the endocrine gene that is inhibited or induced is NGN3. In another aspect, Activin A and Wnt3A are used alone or in combination to inhibit endocrine expression, preferably to inhibit NGN3 expression prior to production of pancreatic foregut endoderm cells, or preferably during stage 3. In one aspect, a gamma secretase inhibitor, preferably R044929097 or DAPT, is used in the culture system to induce expression of endocrine gene expression after production of PEC, or preferably during stages 5, 6 and/or 7.

An in vitro cell culture comprising endocrine cells wherein at least 5% of the human cells express an endocrine marker selected from the group consisting of, insulin (INS), NK6 homeobox 1 (NKX6.1), pancreatic and duodenal homeobox 1 (PDX1), transcription factor related locus 2 (NKX2.2), paired box 4 (PAX4), neurogenic differentiation 1 (NEUROD), forkhead box A1 (FOXA1), forkhead box A2 (FOXA2), snail family zinc finger 2 (SNAIL2), and musculoaponeurotic fibrosarcoma oncogene family A and B (MAFA and MAFB), and does not substantially express a marker selected from the group consisting of neurogenin 3 (NGN3), islet 1 (ISL1), hepatocyte nuclear factor 6 (HNF6), GATA binding protein 4 (GATA4), GATA binding protein 6 (GATA6), pancreas specific transcription factor 1a (PTF1A) and SRY (sex determining region Y)-9 (SOX9), wherein the endocrine cells are unipotent and can mature to pancreatic beta cells.

### In Vivo Nude Rate Study to Evaluate Functional Response

The encapsulation devices were loaded ex vivo with about 6-7×10⁶ cells (or about 20 μL) of pancreatic progenitor cells as described in at least the teachings of U.S. Pat. No. 8,278,106 to Martinson, et. al. After being held in media for less than 24-96 hours, two devices were implanted subcutaneously in each male immunodeficient athymic nude rat. The pancreatic progenitor cells were allowed to develop and mature in vivo and functional performance of the grafts was measured by performing glucose stimulated insulin secretion (GSIS) assays at 12, 16, 20 and 23-24 weeks post-implant.

### GSIS Assay and Measurement of C-peptide Secretion

Animals that had been implanted with encapsulated pancreatic progenitor cells underwent glucose stimulated insulin secretion assays at 12, 16, 20 and 23-24 weeks post device implantation to monitor graft function. Animals were fasted for 4-16 hours and blood samples were taken via jugular vein venupuncture prior to glucose administration at a dose of 3 g/kg body weight via intraperitoneal injection of a sterile 30% glucose solution. Blood samples were again drawn at 90 minutes, or 60 and 90 minutes, or 30 and 60 minutes after glucose administration. Serum was separated from the whole blood and then assayed for human c-peptide using a commercially available ELISA kit (Mercodia, catalog #10-1141-01, Uppsala Sweden). Beta-cells co-release c-peptide with insulin from pro-insulin in an equimolar ratio and c-peptide is measured as a surrogate for insulin secretion due to its longer half-life in blood.

### Nude Rat Explant Histology

At indicated time points post implant, nude rats were euthanized and devices were explanted. Excess tissue was trimmed away and devices were placed in neutral buffered 10% formalin for at least about 6-30 hours. Fixed devices were processed for paraffin embedding in a Leica Biosystems ASP300S tissue processor. Processed devices were cut into 4-6 pieces of approximately 5 mm each and embedded together in paraffin blocks. Multiple 3-10 micron cross sections were cut from each block, place on slides and stained with hematoxylin and eosin (H&E). Images of the slides were captured using a Hamamatsu Nanozoomer 2.0-HT Digital Slide Scanner.

### EXAMPLES

### Example 1

Identical cell encapsulation devices were created with the exception of the biocompatible membrane composites used in each device. One device (Device A) consisted of a two layer biocompatible membrane composite with an ePTFE membrane as the cell impermeable layer and a non-woven polyester as the vascularization layer while the second device (Device B) consisted of a three layer biocompatible membrane composite with an ePTFE membrane as the cell impermeable layer and a non-woven polyester as a vascularization layer but with the addition of another ePTFE membrane as a mitigation layer positioned between the cell impermeable layer and the vascularization layer.

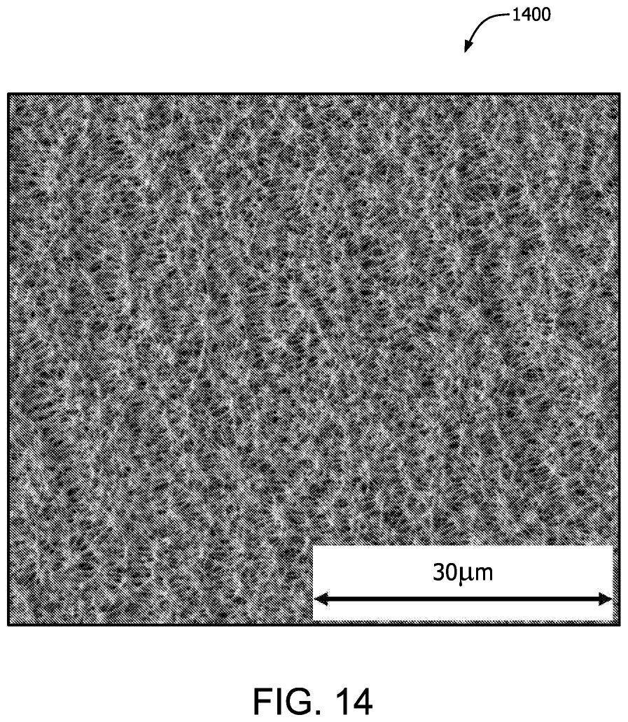

The cell impermeable layer of the first device (Device A) consisted of an ePTFE membrane, which was a commercially available microporous, hydrophilic ePTFE membrane sold under the trade name Biopore® from Millipore (Cork, Ireland). This ePTFE membrane provided a tight, cell impermeable interface and still enabled mass transport of oxygen and nutrients therethrough. A representative scanning electron micrograph (SEM) of the surface of the ePTFE membrane 1400 forming the cell impermeable layer of Device A is shown in FIG. 14. The MPS was determined to be 0.43 microns.

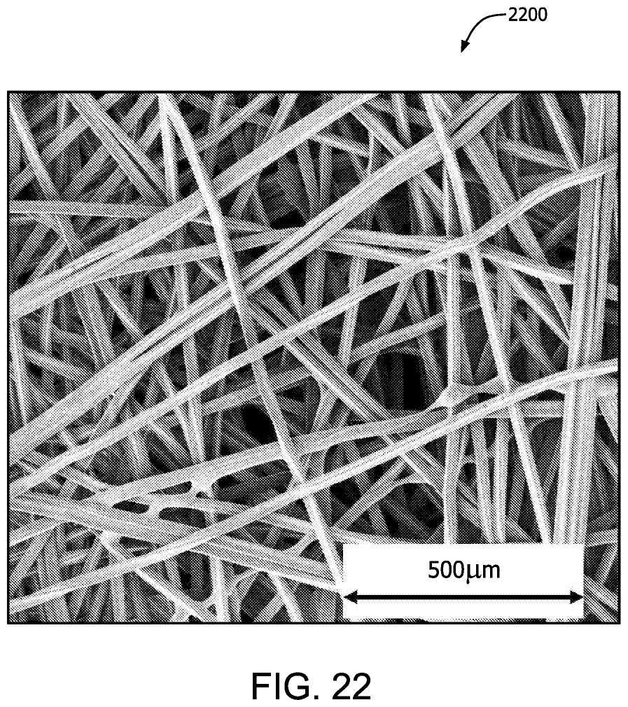

The vascularization layer of the Device A consisted of a commercially available spunbound polyester non-woven material. This vascularization layer was an open layer that provided tissue anchoring and enabled sufficient vascularization of the biocompatible membrane composite. A representative surface microstructure of this vascularization layer is shown in the SEM image in FIG. 22. The relevant properties of the layers of the membrane composite used for Device A are set forth in Table 2.

TABLE 2

| | Layer Function | |
|---|---|---|
| | Cell | |
| | Impermeable | Vascularization |
| | Description | |
| | Biopore ePTFE | PET Non-woven |
| Max Pore Size (μm) | 0.43 | N/A |
| Pore Size (μm) | 0.43 | 101.77 |
| Thickness (μm) | 25.7 | 77.4 |
| Mass (g/m²) | 20.6 | 12.4 |
| Porosity (%) | 63.6 | 92.7 |
| Solid Feature | N/A | 77.9 |
| Spacing (μm) | | |
| Solid Feature Minor | N/A | 28.8 |
| Axis (μm) | | |
| Solid Feature Major | N/A | — |
| Axis (μm) | | |
| Solid Feature Depth | N/A | 27.0 |
| (μm) | | |
| Weakest Axis | 404.2 | 270.4 |
| Tensile Strength | | |
| (N/m) | | |
| Geometric Mean | 37.0 | 6.3 |
| Tensile Strength | | |
| (MPa) | | |
| Composite Bond | — | |
| (kPa) | | |

The two layers (i.e., Cell Impermeable and Vascularization Layers) of Device A were assembled into a composite using a heated lamination process. The fibers of the non-woven material were heated to a temperature above their melt temperature so that they adhered to the ePTFE membrane across the entire surface area of the ePTFE membrane where the fibers of the spunbound non-woven made contact with the surface of the ePTFE membrane. Two examples of laminators used are a Galaxy Flatbed Laminator and a HPL Flatbed Laminator. The conditions were adjusted so that a sufficient pressure and temperature both heated and melted the polyester fibers into the ePTFE membrane at a given run speed. Suitable temperature ranges were identified between 150-170° C., nip pressures between 35 kPA and 355 kPA and run speeds of 1-3 meters per minute.

The second device (Device B) consisted of a three layer biocompatible membrane composite. A first ePTFE membrane (Cell Impermeable Layer) of Device B was formed according to the teachings of U.S. Pat. No. 3,953,566 to Gore. The MPS of this cell impermeable tight layer was determined to be 0.18 microns.

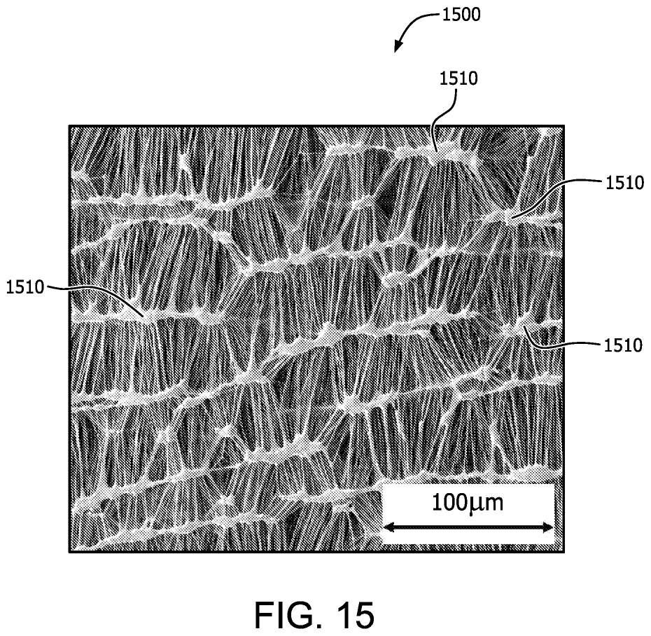

A second ePTFE membrane (Mitigation Layer) of Device B was prepared according to the teachings of U.S. Pat. No. 5,814,405 to Branca, et al. During machine direction (MD) expansion, a fluorinated ethylene propylene (FEP) film was applied to the second ePTFE membrane. Through subsequent co-processing of the second ePTFE membrane and FEP through the machine direction (MD) expansion and transverse direction (TD) expansion, the FEP became discontinuous on the second ePTFE membrane as per the teachings of WO 94/13469 to Bacino. The SEM image shown in FIG. 15 is a representative image of the second ePTFE membrane surface 1500 with a discontinuous layer of FEP 1510 thereon.

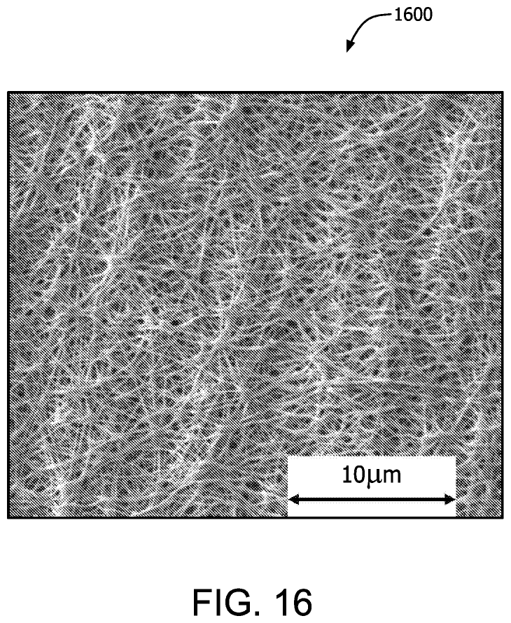

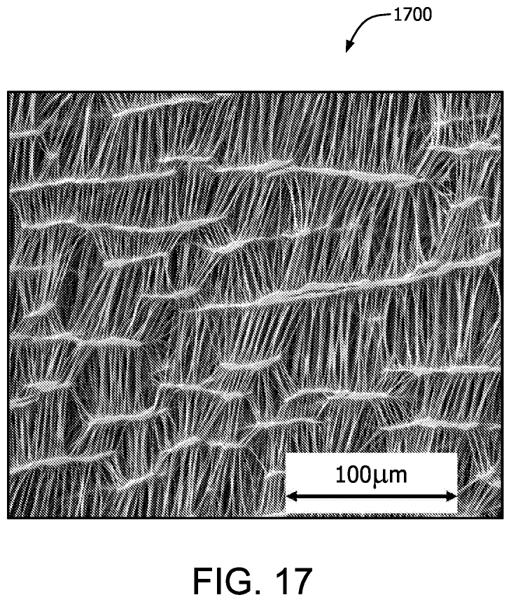

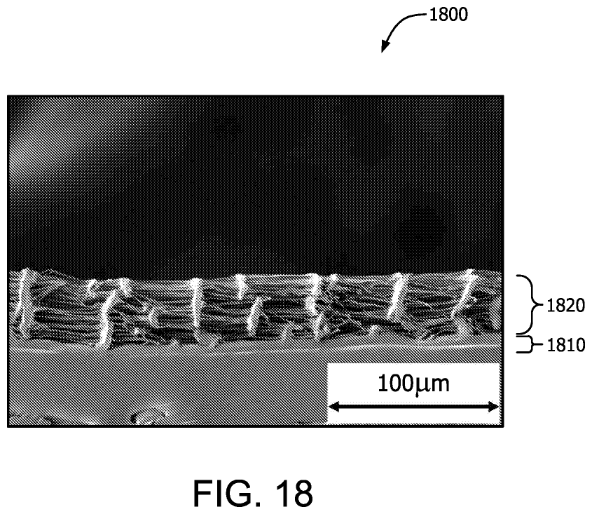

The second ePTFE layer including the discontinuous layer of FEP thereon was laminated to the first ePTFE layer by bringing the materials (with the FEP positioned between the two ePTFE membranes) into contact at a temperature above the melting point of the FEP. The two ePTFE membranes were left unrestrained in the transverse direction during lamination. The laminate was then transversely expanded above the melting point of polytetrafluoroethylene (PTFE) such that each ePTFE layer was returned to its width prior to any necking sustained through lamination. The composite was subsequently rendered hydrophilic per the teachings of U.S. Pat. No. 5,902,745, to Butler, et al. The SEM image shown in FIG. 16 is a representative image of the node and fibril microstructure of the first ePTFE membrane 1600 (Cell Impermeable Layer). The SEM image shown in FIG. 17 is a representative image of the node and fibril microstructure of the second ePTFE membrane 1700 (Mitigation Layer). The SEM image shown in FIG. 18 is a representative image of the cross-section of the two-layer composite 1800 (i.e., the first ePTFE membrane 1810 (Cell Impermeable Layer) and the second ePTFE membrane 1820 (Mitigation Layer)). The nodes within the ePTFE membrane of the second layer served as solid features of the mitigation layer within the biocompatible membrane composite. The solid feature spacing was determined to be 25.7 microns.

The vascularization layer in Device B was the same as Device A and consisted of a commercially available spunbound polyester non-woven material. A representative surface microstructure of this vascularization layer is shown in the SEM image in FIG. 22. The vascularization layer in Device B of this composite was placed on the surface of the mitigation layer and was not permanently or otherwise adhered until the manufacturing of final device form welded all three layers of the biocompatible membrane composite together.

Each individual layer of the biocompatible membrane composite used for Device B was evaluated and characterized for the relevant parameters necessary for the function of each layer. The methods used for the characterization of relevant parameters were performed in accordance with the methods set forth above. Parameters for layers are marked as “N/A” if they are not relevant for that layer's specific function. Parameters for layers are marked as “-” if they are practically unobtainable as a result of how the layers of the composite were processed. The results are summarized in Table 3.

TABLE 3

| | Layer Function | | |
|---|---|---|---|
| | Cell | FBGC | |
| | Impermeable | Mitigation | Vascularization |
| | Description | | |
| | ePTFE | ePTFE | PET |
| | Tight | Open | Non- |
| | Layer | Layer | woven |
| Max Pore Size | 0.18 | — | N/A |
| (microns) | | | |
| Pore Size | 0.34 | 8.06 | 101.77 |
| (microns) | | | |
| Thickness | 6.1 | 44.6 | 77.4 |
| (microns) | | | |
| Mass (g/m²) | 3.8 | 6.2 | 12.4 |
| Porosity (%) | 71.7 | 93.7 | 92.7 |
| Solid Feature | N/A | 24.2 | 77.9 |
| Spacing (microns) | | | |
| Solid Feature | N/A | 4.7 | 28.8 |
| Minor Axis | | | |
| (microns) | | | |
| Solid Feature | N/A | 31.9 | — |
| Major Axis | | | |
| (microns) | | | |
| Solid Feature | N/A | 11.5 | 27.0 |
| Depth (microns) | | | |

| | | |
|---|---|---|
| Weakest Axis | 768.8 | 270.4 |
| Tensile Strength | | |
| (N/m) | | |
| Geometric Mean | 22.8 | 6.3 |
| Tensile Strength | | |
| (MPa) | | |
| Composite Bond | 1231.9 | — |
| (kPa) | | |

Next, to form each of these composite membranes into a device form, a polycarbonate urethane film (i.e., thermoplastic film) was obtained to create a perimeter seal around the components of the devices during welding. A filling tube of the same material as the thermoplastic film (i.e., a polycarbonate urethane) having an outer diameter of 1.60 mm and an inner diameter of 0.889 mm was then obtained. In addition, a reinforcing mechanical support (i.e. reinforcing component) was obtained. In particular, the reinforcing mechanical support was a polyester monofilament woven mesh with 120 micron fibers spaced approximately 300 microns from each other. The stiffness of the reinforcing mechanical support layer was determined to be 0.097 N/cm. A representative surface SEM of this external reinforcing component 5200 can be seen in FIG. 52.

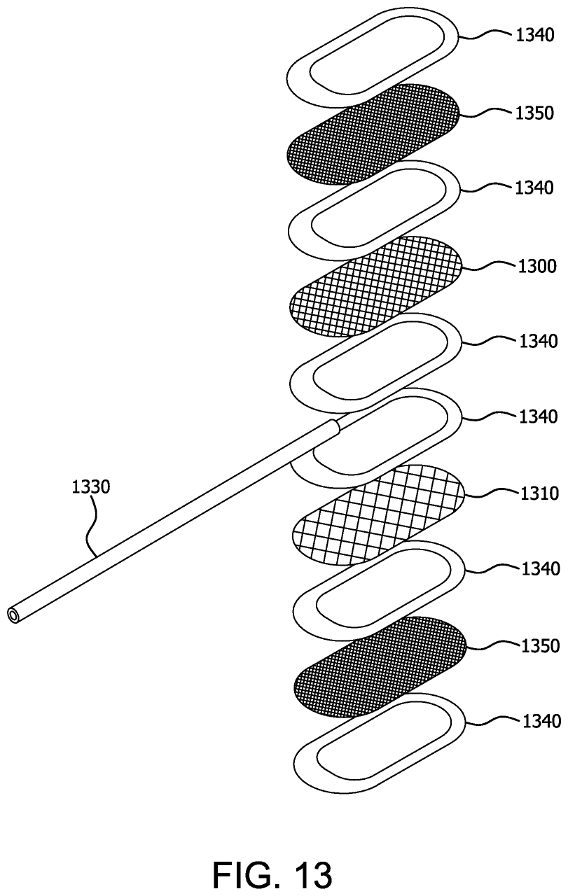

The biocompatible membrane composites of Device A and Device B were then each formed into identical cell encapsulation devices having the configuration shown generally in FIG. 12A. The biocompatible membrane composites of Device A and Device B were first cut to an approximate 22 mm×11 mm oval outer dimension size using a laser cutting table. A thermoplastic weld film (i.e., a polycarbonate urethane film) was cut into oval ring profiles with a 2 mm width. The biocompatible membrane composites, polycarbonate urethane film, and polyester mesh (reinforcing component) were placed in an intercalating stack-up pattern depicted in FIG. 13. This intercalating stack-up pattern of the components allowed for a perimeter seal to be formed by melting the thermoplastic weld film (i.e., polycarbonate urethane film) to bond the two opposing membrane composites and outer polyester mesh (reinforcing material) around the perimeter. The layers forming Device A and Device B were stacked symmetrically opposing the filling tube such that the cell impermeable tight layer of the biocompatible membrane composite was facing internally towards the inner lumen for Device B.

An exploded view of the encapsulation device is shown in FIG. 13. As shown in FIG. 13, the cell encapsulation device is formed from a first biocompatible membrane composite 1300 sealed along a portion of its periphery to a second biocompatible membrane composite 1310 along a portion of its periphery by adhering the two biocompatible membrane composites 1300, 1310 with two weld films 1340. An inner chamber was formed between the biocompatible membrane composites 1300, 1310 with access thereto through a filling tube 1330. Additional weld films 1340 were positioned on each side of the biocompatible membrane composites 1300, 1310 and the reinforcing component 1350.

An integral perimeter seal around the encapsulation device was formed by using an ultrasonic welder (Herrmann Ultrasonics) for Device A and a thermal staking welder (Thermal Press International, Inc.) for Device B. With both processes, thermal or vibrational energy and force were applied to the intercalated stack-up to melt and flow the thermoplastic film (polycarbonate urethane film) above its softening temperature to weld all of the layers together. The biocompatible encapsulation devices were constructed in a two-step welding process where energy or heat was applied from one side such that the first biocompatible membrane composite was integrated into one side of the encapsulation device followed by the second biocompatible membrane composite onto the opposing side of the device. The final suitability of the welds were assessed by testing the devices for integrity using a pressure decay test with a USON Sprint iQ Leak Tester at a test pressure of 5 psi.

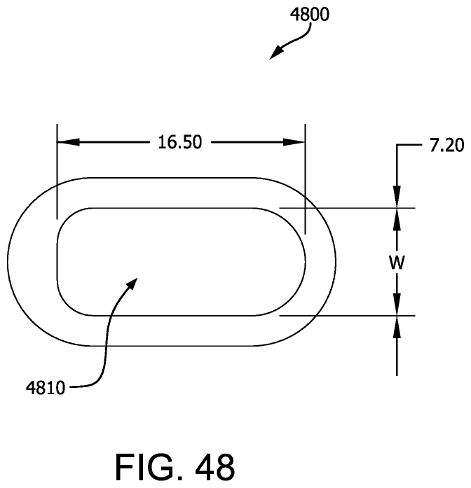

The weld spacing (W) between the perimeter seal around the lumen 4810 of the cell encapsulation device 4800 was 7.2 mm as illustrated in FIG. 48 for both devices. Device A and Device B had the same footprint and are both represented generally by reference numeral 4800.

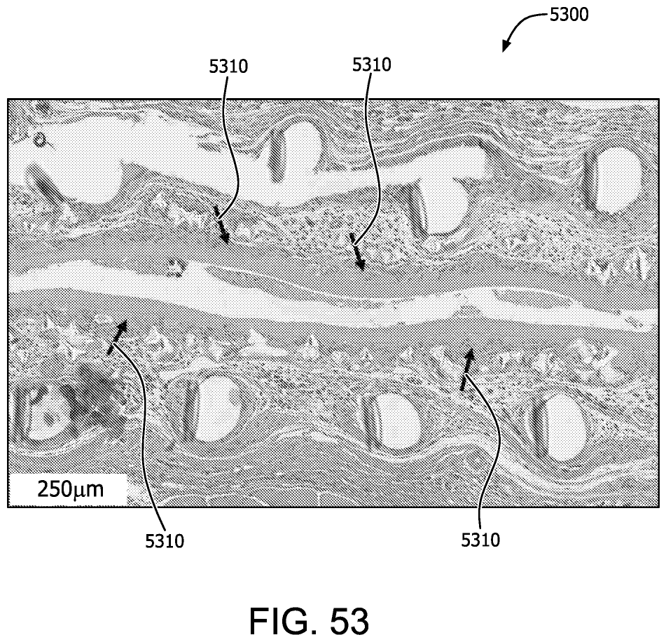

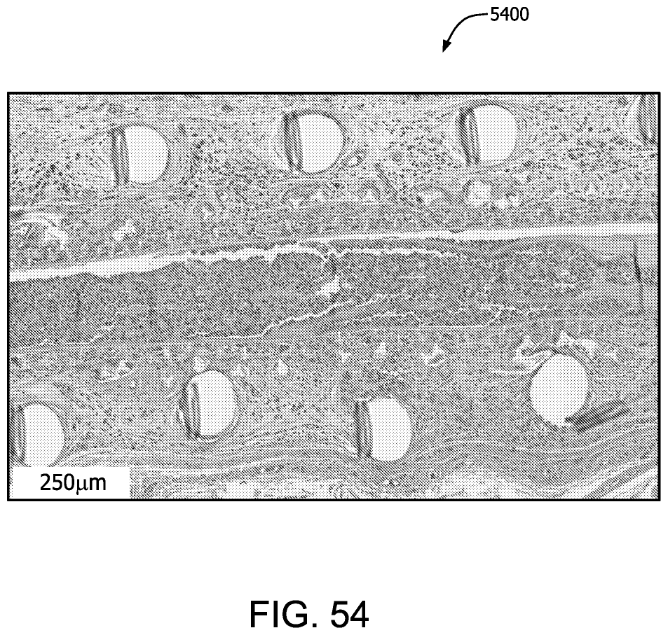

Both Devices were evaluated for functional response in accordance with the In Vivo Nude Rat Study set forth in the Test Methods section above. The functional response of the Devices loaded with cells is shown in Table 4. The results demonstrated a step change in functional response for Device B (which included a mitigation layer) in comparison to that of Device A (with no mitigation layer). A representative histology image 5300 of Device A is shown in FIG. 53 and illustrates the presence of foreign body giant cells 5310 and very few blood vessels at the cell impermeable layer interface thereby resulting in very few viable encapsulated cells. Comparatively, the histology image 5400 of Device B is shown in FIG. 54 and does not show the presence of foreign body giant cells at the cell impermeable layer and instead shows many blood vessels at this location resulting in viable cells consuming the entire lumen. From the evaluation of these histology images, it can be concluded that the presence of the solid features in the mitigation layer of Device B successfully mitigated the formation of foreign body giant cells on the cell impermeable layer and thereby resulted in the step change in functional response, thereby demonstrating the importance of the presence of a mitigation layer in a cell encapsulation device.

TABLE 4

| | Mean Human c-peptide | | | | | | | | | |
|---|---|---|---|---|---|---|---|---|---|---|
| | serum levels for each time point | | | | | | | | Sample | |
| | 12 | | 16 | | 20 | | 23-24 | | size (n) for | |
| | weeks | | weeks | | weeks | | weeks | | each time point | |
| GSIS Time (min) | 0 | 90 | 0 | 90 | 0 | 90 | 0 | 90 | # animals | # devices |
| Device A | 12** | 48 | 30 | 98 | 29 | 154 | 62 | 124 | 5 | 10 |
| Device B | 26 | 297.7 | 43 | 490 | 91 | 594.7 | 118 | 615 | 6 | 12 |

**rats were not fasted prior to GSIS assay

### Example 2

An alternate membrane composite containing three (3) layers was used to construct the cell encapsulation device form described in Example 1. The only difference was that the device geometry was modified to intentionally vary the weld spacing between the perimeter seal of the device lumen.

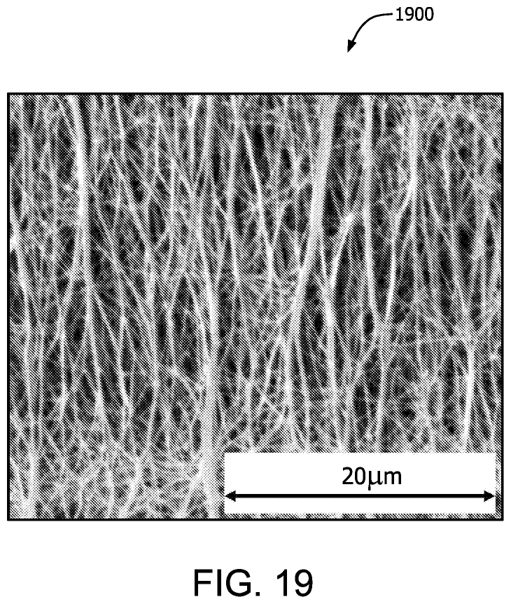

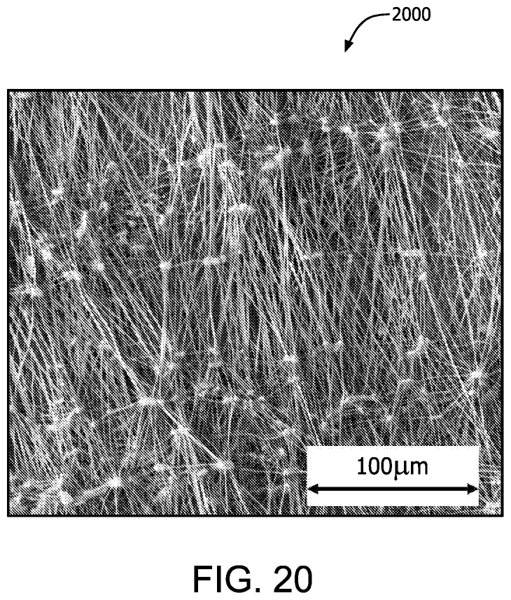

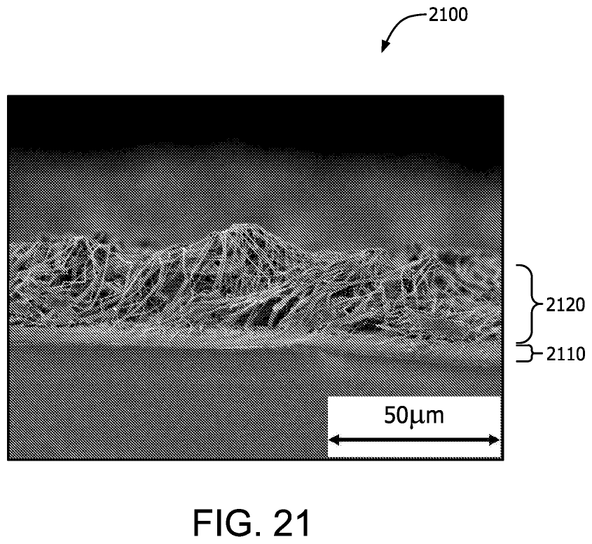

A biocompatible membrane composite having three distinct layers was constructed. First, a two-layer ePTFE composite was prepared by layering and then co-expanding a first ePTFE layer consisting of a dry, biaxially-expanded membrane (Cell Impermeable Layer) prepared according to the teachings of U.S. Pat. No. 3,953,566 to Gore and a second ePTFE layer consisting of a paste extruded calendered tape (Mitigation Layer) prepared according to the teachings of U.S. Pat. No. 3,953,566 to Gore. The two-layer ePTFE composite was biaxially expanded and then rendered hydrophilic according to the teachings of U.S. Pat. No. 5,902,745, to Butler, et al. The first ePTFE layer provided a tight, cell impermeable interface while still enabling mass transport of oxygen and nutrients. A representative surface microstructure of the first ePTFE layer 1900 (Cell Impermeable Layer) is shown in the SEM image of FIG. 19. The pore size of this cell impermeable tight layer was determined to be 0.35 microns. A representative surface microstructure of the second ePTFE membrane 2000 (Mitigation Layer) is shown in FIG. 20. A representative cross-section showing the microstructure of the composite 2500 including the first ePTFE membrane 2510 (Cell Impermeable Layer) and the second ePTFE membrane 2520 (Mitigation Layer) is shown in the SEM image of FIG. 21.

An additional third layer was included in this biocompatible membrane composite as a supplemental vascularization layer. This third layer was a commercially available spunbound polyester non-woven material. A representative surface microstructure of this third, spunbond polyester non-woven material 2200 (Vascularization Layer) is shown in the SEM image in FIG. 22. This third layer was assembled into a biocompatible membrane composite with the first and second ePTFE membranes (i.e., the two-layer ePTFE membrane composite) by placing the spunbound polyester non-woven on the top of the second ePTFE membrane 2120 (Mitigation Layer) of the two-layer ePTFE membrane composite during device manufacturing and was welded at the perimeter with thermoplastic weld rings during device assembly as described in Example 1.

Each individual layer of the biocompatible membrane composite was evaluated and characterized for the relevant parameters necessary for the function of each layer. The methods used for this characterization of relevant parameters were performed in accordance with the test methods set forth above. Parameters for layers are marked as “N/A” if they are not relevant for that layer's specific function. Parameters for layers are marked as “-” if they are practically unobtainable as a result of how the layers of the composite were processed. The results are summarized in Table 5.

TABLE 5

| | Layer Function | | |
|---|---|---|---|
| | Cell | FBGC | |
| | Impermeable | Mitigation | Vascularization |
| | Description | | |
| | ePTFE | ePTFE | PET |
| | Tight | Open | Non- |
| | Layer | Layer | woven |
| Max Pore Size (microns) | 0.35 | — | N/A |
| Pore Size (microns) | 0.51 | 4.87 | 101.77 |
| Thickness (microns) | 6.6 | 24.7 | 77.4 |
| Mass (g/m²) | 2.3 | 2.2 | 12.4 |
| Porosity (%) | 83.8 | 95.9 | 92.7 |
| Solid Feature Spacing | N/A | 24.4 | 77.9 |
| (microns) | | | |
| Solid Feature Minor Axis | N/A | 4.2 | 28.8 |
| (microns) | | | |
| Solid Feature Major Axis | N/A | 7.5 | — |
| (microns) | | | |
| Solid Feature Depth | N/A | 5.2 | 27.0 |

| | | |
|---|---|---|
| (microns) | | |
| Weakest Axis Tensile | 210.9 | 270.4 |
| Strength (N/m) | | |
| Geometric Mean Tensile | 38.1 | 6.3 |
| Strength (MPa) | | |
| Composite Bond (kPa) | 170.2 | — |

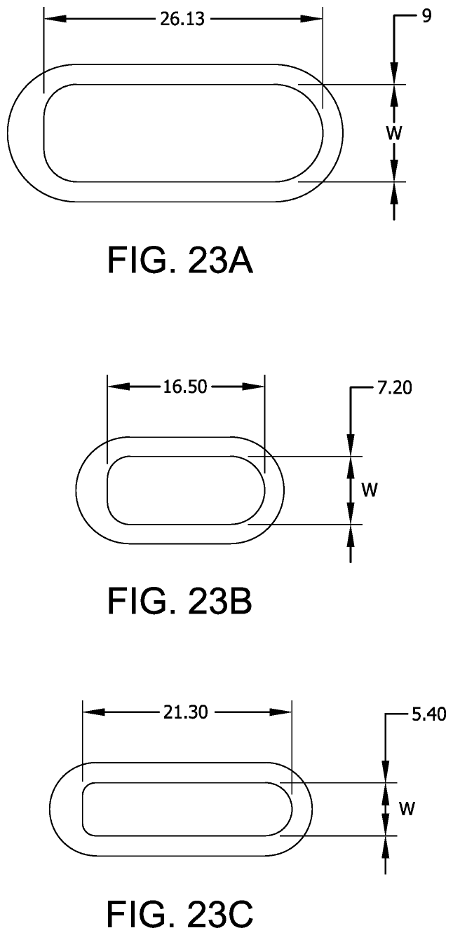

When this biocompatible membrane composite was integrated into a cell encapsulation device as described in Example 1, it included the same external reinforcing component which was a monofilament polyester woven mesh with a stiffness of 0.097 N/cm. The geometry of the device was modified to intentionally vary the weld spacing between the perimeter seal across three different device geometries. Device A shown in FIG. 23A had the largest weld spacing (W) at 9 mm, Device B shown in FIG. 23B had a weld spacing (W) consistent with Example 1 at 7.2 mm, and Device C shown in FIG. 23C had the narrowest weld spacing (W) at 5.4 mm. FIGS. 23 A-C generally shows the geometry of each of these cell encapsulation devices.

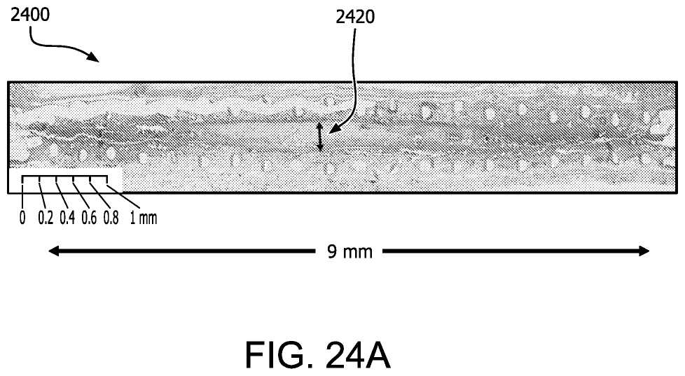

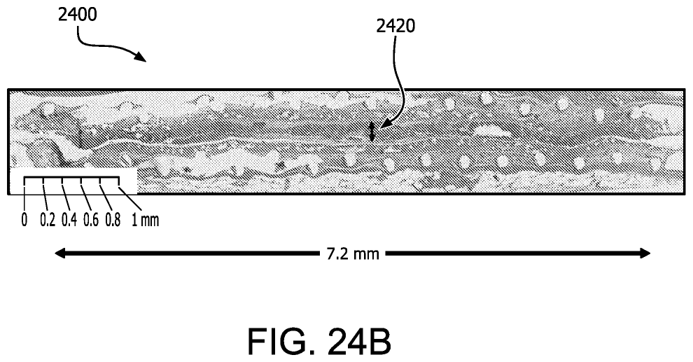

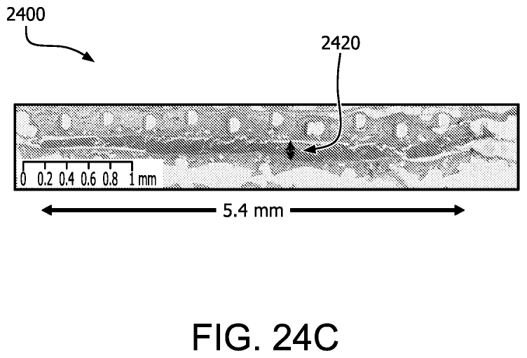

Each encapsulation device was evaluated for maximum oxygen diffusion distance (ODD) at 1 PSI internal pressure and implanted in accordance with the In Vivo Nude Rat study set forth in the Test Methods section set forth above. A summary table of the results is shown in Table 8. The results demonstrated that with a consistent external reinforcing component, the oxygen diffusion distance can be limited by controlling the weld spacing between the perimeter seals of the device. The oxygen diffusion distance was also shown to track with histological observations of graft thickness in the lumen as shown in FIGS. 24A-C. As shown in FIGS. 24A-C, the devices with narrower weld spacing and smaller oxygen diffusion distances demonstrated thinner graft thickness in vivo at 20 weeks as evidenced by the size of the arrows 2420 indicating the maximum graft thickness across the cross-section of the device. Additionally, the functional response of the devices as measured by the GSIS C-peptide response showed a trend of significant increased function with decreased oxygen diffusion distances as shown in Table 6.

TABLE 6

| | | | | | | Mean |
|---|---|---|---|---|---|---|
| | | | | | | Human |
| | | | | | | c-peptide |
| | | | | | | serum |
| | | | | | | levels |
| | External | | | Cell | Maximum | at GSIS |
| | Reinforcing | | In Vitro | Impermeable | Oxygen | time of |
| | Component | Weld | Lumen | Layer | Diffusion | 90 min |
| | Stiffness | Spacing | Expansion | Thickness | Distance | (pM @ |
| Device | (N/cm) | (mm) | (microns) | (microns) | (microns) | 30 wks) |
| Device | 0.097 | 9.0 | 1300 | 4.2 | 654 | 225 |
| A | | | | | | |
| Device | 0.097 | 7.2 | 350 | 4.2 | 179 | 593 |
| B | | | | | | |
| Device | 0.097 | 5.4 | 200 | 4.2 | 104 | 746 |
| C | | | | | | |

### Example 3

A biocompatible membrane composite having three distinct layers was constructed. A first layer formed of an ePTFE membrane (Cell Impermeable Layer) was formed according to the teachings of U.S. Pat. No. 3,953,566 to Gore.

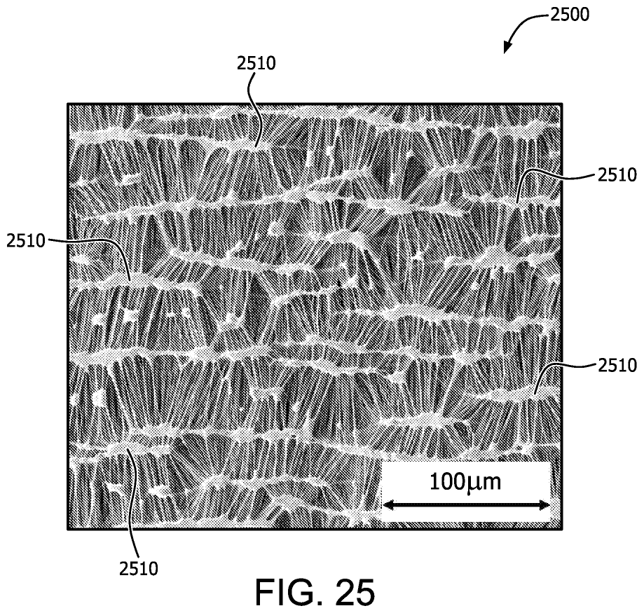

A two-layer composite consisting of a second ePTFE membrane (Mitigation Layer) and a third ePTFE membrane (Vascularization Layer) was formed. The second ePTFE membrane was prepared according to the teachings of U.S. Pat. No. 5,814,405 to Branca, et al. The ePTFE tape precursor of the second ePTFE layer was processed per the teachings of U.S. Pat. No. 5,814,405 to Branca, et al. through the below-the-melt MD expansion step. During the below-the-melt MD expansion step of the second ePTFE tape precursor, an FEP film was applied per the teachings of WO/94/13469 to Bacino. The ePTFE tape precursor of the third ePTFE layer was then processed per the teachings of U.S. Pat. No. 5,814,405 to Branca, et al. through an amorphous locking step and above-the-melt MD expansion. During the first below-the-melt MD expansion step of the ePTFE tape precursor, an FEP film was applied per the teachings of WO 94/13469 to Bacino. The expanded ePTFE tape precursor of the third ePTFE membrane was laminated to the expanded ePTFE tape precursor of the second ePTFE membrane such that the FEP side of the third ePTFE tape was in contact with the PTFE side of the ePTFE tape precursor of the second ePTFE membrane. The two-layer composite was then co-expanded in the machine direction and transverse direction above the melting point of PTFE. A representative surface microstructure of the second ePTFE membrane 2500 having thereon FEP 2510 is shown in the SEM image of FIG. 25.

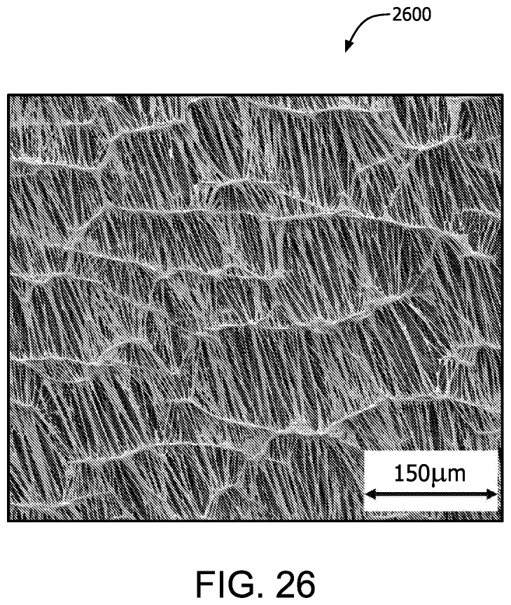

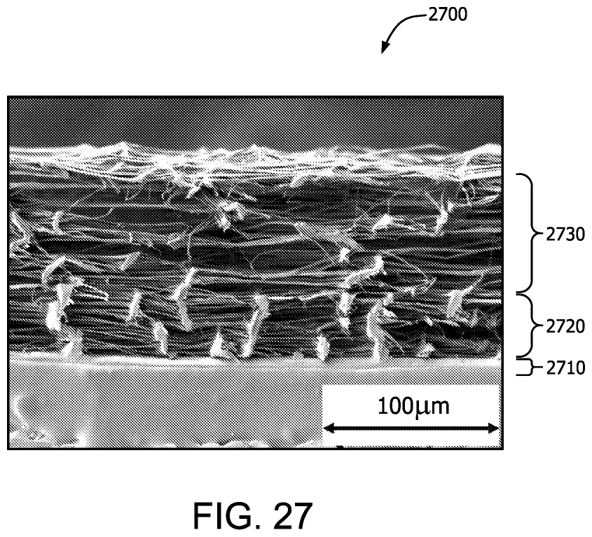

The two-layer composite consisting of the second ePTFE membrane (Mitigation Layer) and third ePTFE membrane (Vascularization Layer) was laminated to the first ePTFE membrane (Cell Impermeable Layer). The side of the second ePTFE membrane comprising a discontinuous layer of FEP thereon was laminated to the first ePTFE layer by first bringing two-layer ePTFE composite into contact with the third ePTFE layer (with the FEP positioned between the two layers) at a temperature above the melting point of the FEP with the ePTFE membranes unrestrained in the transverse direction. The laminate was then transversely expanded above the melting point of PTFE so each layer was returned to its width prior to any necking sustained through lamination. The resulting biocompatible membrane composite was subsequently rendered hydrophilic per the teachings of U.S. Pat. No. 5,902,745 to Butler, et al. The SEM image shown in FIG. 16 is a representative image of the node and fibril microstructure of the first ePTFE membrane (Cell Impermeable Layer). The SEM image shown in FIG. 26 is a representative image of the node and fibril microstructure of the third ePTFE membrane 2600 (Vascularization Layer). The SEM image shown in FIG. 27 is a representative image of the cross-section 2700 of the three layer biocompatible membrane composite including the first ePTFE membrane 3710 (Cell Impermeable Layer), the second ePTFE membrane 3720 (Mitigation Layer), and the third ePTFE membrane 3730 (Vascularization Layer).

Each individual layer of the biocompatible membrane composite was evaluated and characterized for the relevant parameters necessary for the function of each layer. The methods used for this characterization of relevant parameters were performed in accordance with the test methods set forth above. Parameters for layers are marked as “N/A” if they are not relevant for that layer's specific function. Parameters for layers are marked as “-” if they are practically unobtainable as a result of how the layers of the composite were processed. The resulting properties of the biocompatible membrane composite are shown in Table 7.

TABLE 7

| | Layer Function | | |
|---|---|---|---|
| | Cell | FBGC | |
| | Imper- | Miti- | |
| | meable | gation | Vascularization |
| | Description | | |
| | ePTFE | ePTFE | ePTFE |
| | Tight | Open | Open |
| | Layer | Layer | Layer |
| Max Pore Size (microns) | 0.19 | — | — |
| Pore Size (microns) | 0.34 | 8.06 | 10.15 |
| Thickness (microns) | 6.7 | 42.8 | 80.5 |
| Mass (g/m²) | 3.0 | 4.8 | 5.5 |
| Porosity (%) | 79.3 | 94.9 | 96.9 |
| Solid Feature Spacing (microns) | N/A | 24.2 | 58.4 |
| Solid Feature Minor Axis | N/A | 4.7 | 8.6 |
| (microns) | | | |
| Solid Feature Major Axis | N/A | 31.9 | 83.7 |
| (microns) | | | |
| Solid Feature Depth (microns) | N/A | 16.3 | 11.7 |

| | |
|---|---|
| Weakest Axis Tensile Strength | 814.7 |
| (N/m) | |
| Geometric Mean Tensile | 13.3 |
| Strength (MPa) | |
| Composite Bond (kPa) | 235.0 |

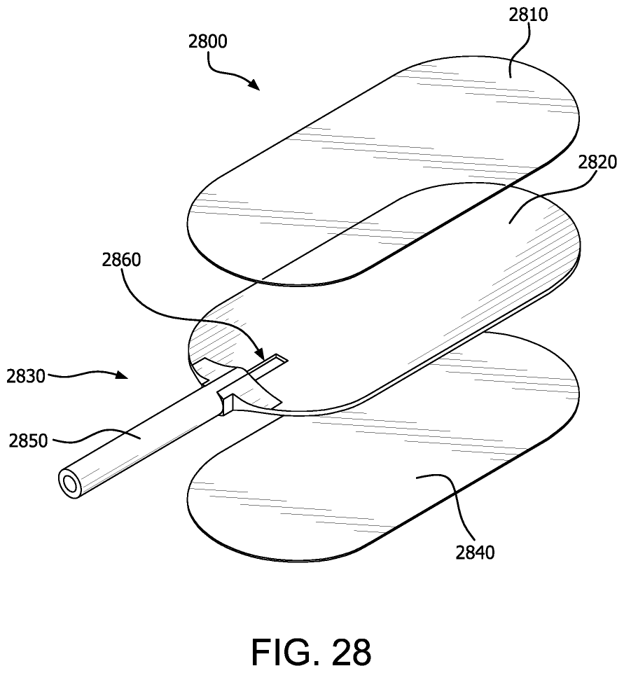

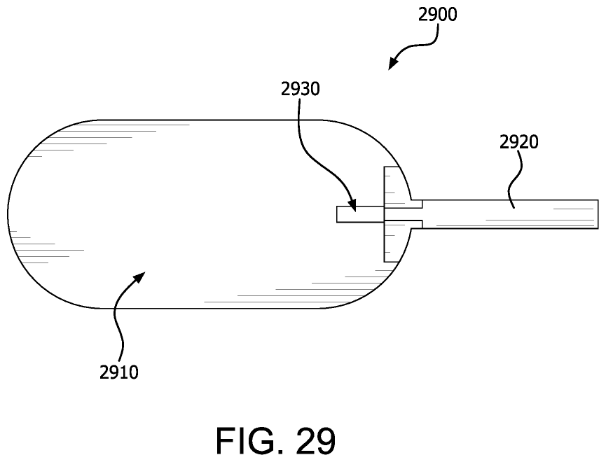

Two identical biocompatible membrane composites were integrated into a planar device 2800 that included an internal reinforcing component 2830 as shown generally in FIG. 28. The planar cell encapsulation device described in this Example differs from the previously described devices (i.e., the devices in Examples 1-2) in that the planar device is based on a reinforcing component 2820 (depicted in FIG. 28) that is an internal reinforcing component located adjacent to the cell impermeable layers of the two biocompatible membrane composites. The reinforcing component 2820 is located within the lumen of the device (e.g., as an endoskeleton) as opposed to the external reinforcing component that was provided by the woven polyester mesh(es) in the previous Examples. The reinforcing component 2900 included a reinforcing insert 2910 and an integrated filling tube 2920 with a flow through hole 2930 to access both sides of the reinforcing component 2900.

The reinforcing component was constructed by placing a sheet of a fluorothermoplastic terpolymer of TFE, HFP, and VDF into a mold cavity and pressing the terpolymer in an heated press (Wabash C30H-15-CPX) set at a temperature above the softening temperature of the polymer so that it conforms to the final dimension and shape. The resulting reinforcing component had a thickness of approximately 270 microns and a stiffness of 0.6 N/cm.

Two biocompatible membrane composites were cut to approximately 1″×2″ (2.54 cm×5.08 cm) and arranged on both sides of the reinforcing component with the Cell Impermeable Layer of each membrane composite facing inwardly towards the lumen and the reinforcing component. An exploded view of the individual components of the planar device is shown in FIG. 28.

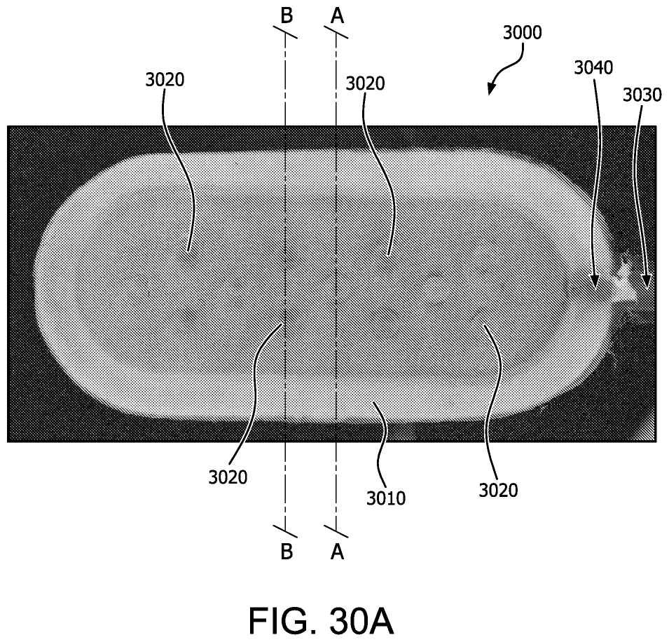

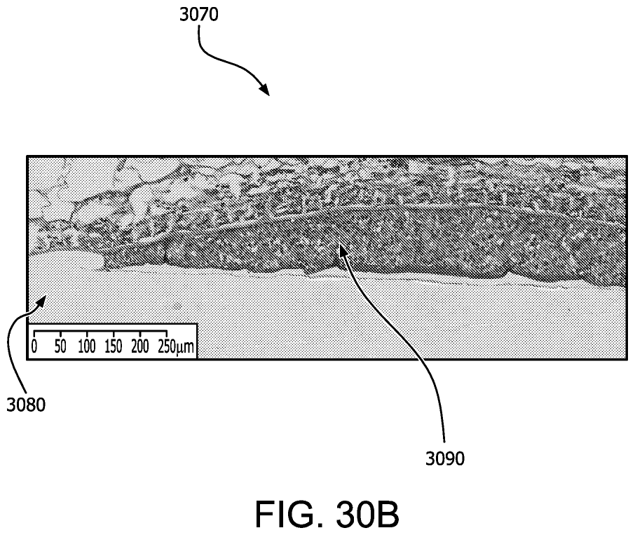

The planar device is shown in FIG. 30. To create the planar device 3000, a weld was formed by compressing the material stack 2800 shown in FIG. 28 using an impulse welder along the perimeter and applying a temperature and pressure such that the thermoplastic softened enough to form a bond into each composite membrane. During welding, a steel mandrel (not illustrated) was put in the filling tube 3030 to prevent the filling tube 3030 from being welded shut during heating. Internal points of the reinforcing planar component 3000 were bonded to each membrane composite surface by applying light manual pressure with a thermal head to create internal point bonds 3020 of approximately 1 mm diameter spaced at least 1.45 mm apart at 12 locations on each side. The integrity of the welds were evaluated for suitability by testing for the presence of leaks visually detected as a stream of bubbles when submerged in isopropyl alcohol at an internal pressure of 5 psi.

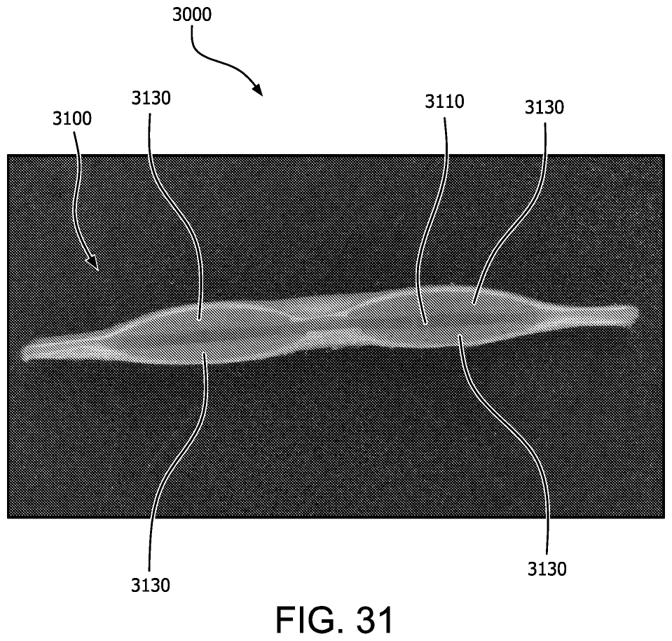

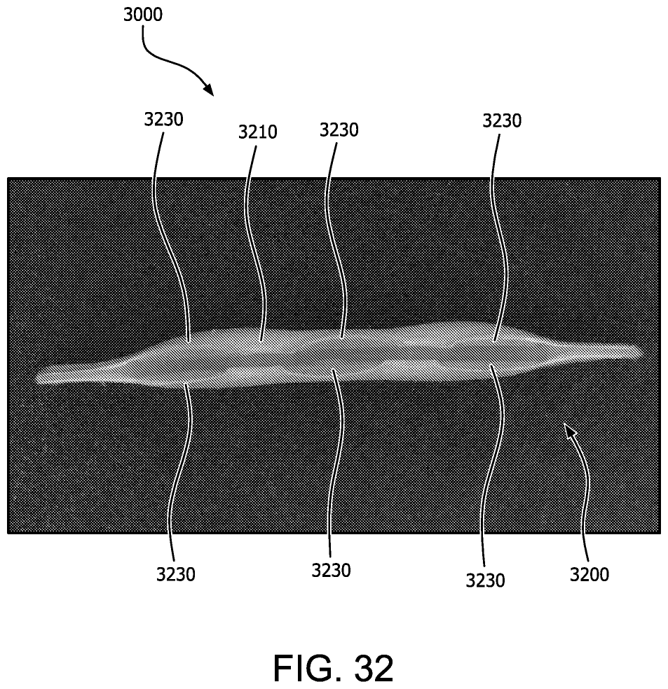

Turning to FIG. 30, the internal geometry of the reinforcing component 3010 and internal lumen 3030 of the planar device 3000 is shown in cross-section. The internal geometry of the reinforcing component 3010 and internal lumen 3030 is shown in FIGS. 31 and 32. FIG. 31 depicts a cross-section of the planar device 3000 taken along line A-A showing a single point bond 3120 and the lumen 3130. FIG. 32 is a cross-section image of the planar device 3000 taken along line B-B showing two point bonds 3220 and the lumen 3230. The finished planar device shown in FIG. 30 was filled with a low viscosity silastic to allow for better visualization and imaging of the reinforcing component 3110 shown in FIGS. 31 and 3210 shown in FIG. 32.

The planar device 3000 was evaluated for oxygen diffusion distance (ODD) at 1 PSI and then implanted to evaluate the histological response in accordance with the Nude Rat Explant Histology set forth in the Test Methods section above. It was determined that the planar device 3000 had a maximum oxygen diffusion distance of 194 microns at 1 PSI. The results also demonstrated that the oxygen diffusion distance can be controlled and limited through the inclusion of a reinforcing component 3040 positioned in the lumen of the planar device. The control of oxygen diffusion distance can be observed in the representative histological cross section as shown in a representative cross-section of the planar device 3000 shown in FIG. 30B. It was concluded from the histological evaluation that the oxygen diffusion distance of the planar device 3000 successfully enabled in vivo cell viability at 24 weeks as evidenced viable encapsulated cells 3050 in FIG. 30B.

### Comparative Example 1

The biocompatible membrane composite and device described in Example 3 were used with the exception that there were no internal points bonding the reinforcing planar component to the biocompatible membrane composite surface. The purpose of this device embodiment is to provide a comparative example to demonstrate the impact of the internal point bonds in maintaining adequate oxygen diffusion distances.

The device was evaluated for oxygen diffusion distance (ODD) at 1 PSI in accordance with the Oxygen Diffusion Distance method set forth in the Test Methods section above. The device made for this comparative example without point bonds resulted in an maximum Oxygen Diffusion Distance of 1159 microns. This maximum oxygen diffusion distance is compared to the maximum diffusion distance of 194 microns in Example 3 when internal point bonds are present. These results demonstrated that the oxygen diffusion distance can be controlled and limited through the inclusion of an internal reinforcing component positioned in the lumen of a planar device and bonded to the biocompatible composite membrane.

### Example 4

A device was constructed as described in Example 3 with the exception of the membrane composite used and the geometry of the internal reinforcing component used.

A biocompatible membrane composite having three distinct layers was constructed. First, a two-layer ePTFE composite was prepared by layering and then co-expanding a first ePTFE layer consisting of a dry, biaxially-expanded membrane (Cell Impermeable Layer) prepared according to the teachings of U.S. Pat. No. 3,953,566 to Gore and a second ePTFE layer consisting of a paste extruded, calendered tape (Mitigation Layer) prepared according to the teachings of U.S. Pat. No. 3,953,566 to Gore. The two-layer ePTFE composite (Cell Impermeable Layer/Mitigation Layer) was biaxially expanded to form the final composite structure.

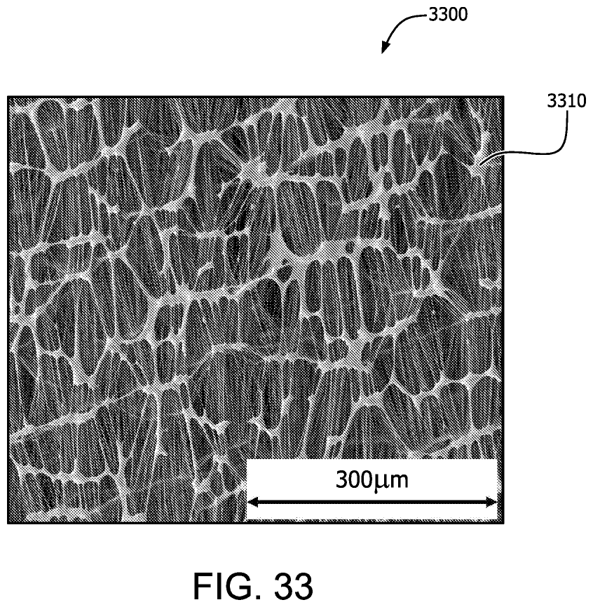

The third layer (Vascularization Layer) was prepared according to the teachings of U.S. Pat. No. 5,814,405 to Branca, et al. During an initial machine direction (MD) expansion step, a fluorinated ethylene propylene (FEP) film was applied to the third ePTFE membrane. Through subsequent co-processing of the third ePTFE membrane and FEP through the machine direction (MD) expansion and transverse direction (TD) expansion, the FEP became discontinuous on the surface of the third ePTFE membrane per the teachings of WO/94/13469 to Bacino. FIG. 33 is a representative image of the surface 3300 of the third ePTFE layer with a discontinuous layer of FEP 3310 thereon.

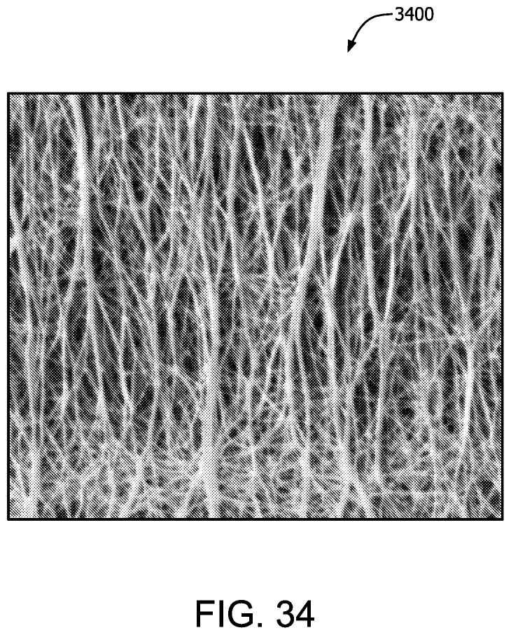

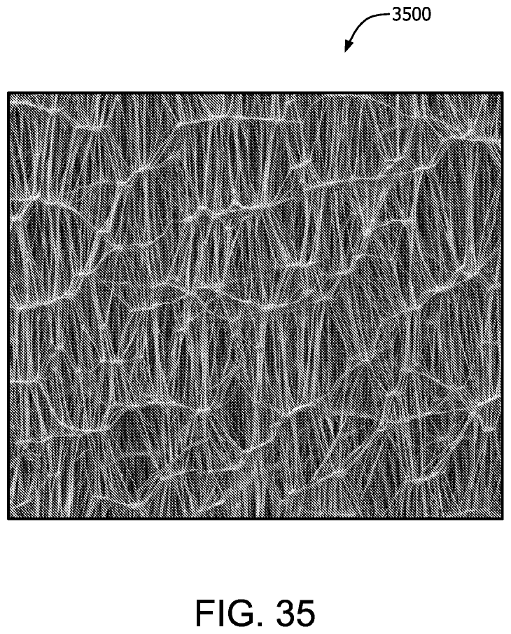

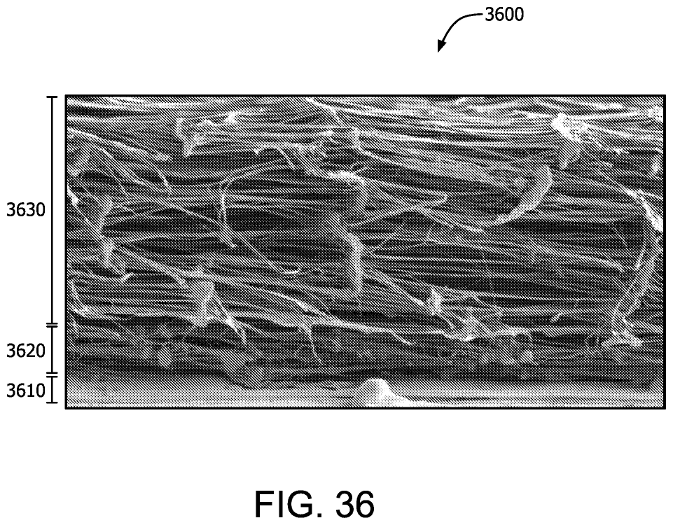

The third ePTFE membrane was laminated to the two-layer ePTFE composite. The side of the third ePTFE layer having thereon the discontinuous layer of FEP was laminated to the second ePTFE membrane (of the two-layer ePTFE composite) by first bringing the third ePTFE membrane into contact with the second ePTFE membrane of the two-layer ePTFE composite (with the FEP positioned between the second and third ePTFE membranes) at a temperature above the melting point of the FEP. The ePTFE membranes were unrestrained in the transverse direction during lamination. The laminate was then transversely expanded above the melting point of PTFE so that each layer was returned to its original width prior to any necking sustained through lamination. The resulting biocompatible membrane composite was subsequently rendered hydrophilic per the teachings of U.S. Pat. No. 5,902,745 to Butler, et al. The SEM image shown in FIG. 34 is a representative image of the node and fibril microstructure 3400 of one side (i.e., Cell Impermeable Layer) of the two-layer ePTFE composite. The SEM image shown in FIG. 35 is a representative image of the node and fibril microstructure of the third membrane 3500 (Vascularization Layer). The SEM image shown in FIG. 36 is a representative image of the cross-section of the three layer biocompatible membrane composite 3600 including the first ePTFE membrane 3610 (Cell Impermeable Layer), the second ePTFE membrane 3620 (Mitigation Layer) and the third ePTFE membrane 3630 (Vascularization Layer). The resulting properties of the biocompatible membrane composite are shown in Table 8.

TABLE 8

| | Layer Function | | |
|---|---|---|---|
| | Cell | FBGC | Vascu- |
| | Impermeable | Mitigation | larization |
| | Description | | |
| | ePTFE | ePTFE | ePTFE |
| | Tight | Open | Open |
| | Layer | Layer | Layer |
| Max Pore Size (microns) | 0.41 | — | — |
| Pore Size (microns) | 0.51 | 4.87 | 11.09 |
| Thickness (microns) | 8.0 | 35.5 | 94.9 |
| Mass (g/m²) | 3.5 | 2.3 | 7.4 |
| Porosity (%) | 79.9 | 97.2 | 96.5 |
| Solid Feature Spacing | N/A | 24.4 | 46.5 |
| (microns) | | | |
| Solid Feature Minor | N/A | 4.2 | 6.8 |
| Axis (microns) | | | |
| Solid Feature Major | N/A | 7.5 | 26.6 |
| Axis (microns) | | | |
| Solid Feature Depth | N/A | 4.6 | 11.6 |
| (microns) | | | |

| | |
|---|---|
| Weakest Axis Tensile | 303.3 |
| Strength (N/m) | |
| Geometric Mean | 20.2 |
| Tensile Strength (MPa) | |
| Composite Bond (kPa) | 81.8 |

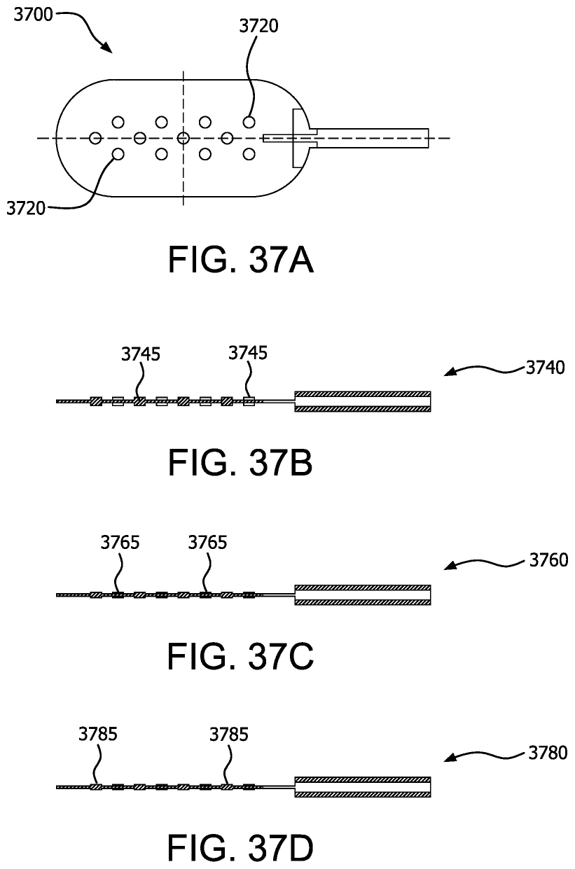

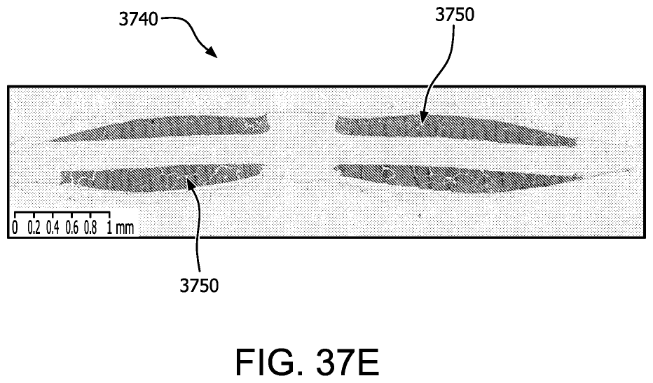

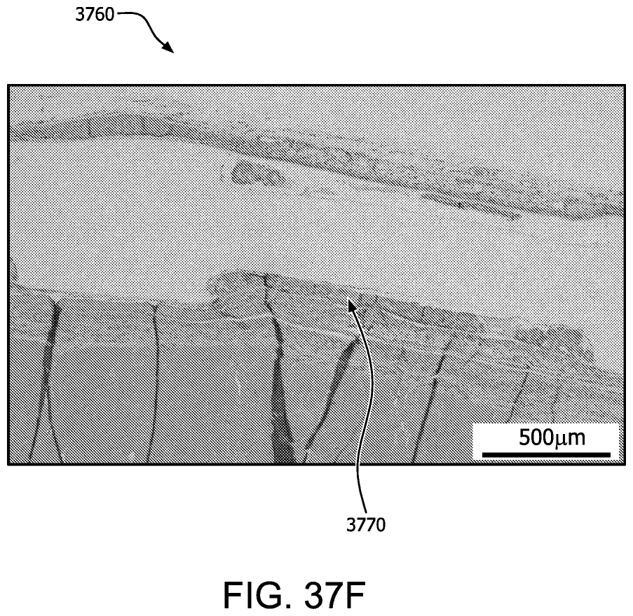

When this biocompatible membrane composite was integrated into a device as described in Example 3, the internal reinforcing component geometry was modified to intentionally vary the height of internal points of the reinforcing planar component (i.e., pillars). FIG. 37A is a top view of a reinforcing component 3700 with pillars 3720. In Device A 3740, as seen in FIG. 37B, the planar device 3740 had a geometry with 250 micron pillars 3745. In Device B 3760, as seen in FIG. 37C, the planar device 3760 had an internal geometry with 150 microns pillars 3765. In Device C 3780, as seen in FIG. 37D, the planar device 3780 had an internal geometry with 75 microns pillars 3785. It should be noted that the bonding of the membrane to the pillars will change the final pillar heights due to compression and polymer flow into the membrane structure, and/or excess polymer flash outside of the intended bonded region.

Each device was evaluated for Oxygen Diffusion Distance (ODD) at 1 psi in accordance with the Oxygen Diffusion Distance (ODD) test set forth

above in the Test Methods section. A summary of the ODD results is shown in Table 9. The results demonstrated that maximum oxygen diffusion distance can be controlled and limited through the inclusion of a reinforcing component within the lumen of a cell encapsulation device and that the geometry of the reinforcing component of the reinforcing component can be adjusted to target a desired oxygen diffusion distance.

TABLE 9

| | Internal | | Maximum |
|---|---|---|---|
| | Reinforcing | Point | Oxygen |
| | Component | Weld | Diffusion |
| | Stiffness | Spacing | Distance |
| Device | (N/cm) | (mm) | (microns) |
| Device A | 0.6 | 1.45-3.0 | 303.5 |
| Device B | 0.6 | 1.45-3.0 | 258.5 |
| Device C | 0.6 | 1.45-3.0 | 211.5 |

The functional performances of Device A 3740 and Device B 3760 loaded with cells were evaluated in accordance with the Nude Rat Explant Histology set forth in the Test Methods section above. The resulting decrease in oxygen diffusion distance observed with decreased pillar height was also shown to track with the histological observations of graft thickness in the lumen as shown in representative cross-sections of Device A 3740 in FIG. 37B, and Device B 3760 in FIG. 37C. Additionally, it can be concluded from the histological evaluation that the presence of a mitigation layer and oxygen diffusion distances of Device A 3740 and Device B 3760 enabled in vivo cell viability as evidenced by viable cells 3750, 3770 in FIGS. 37E and 37F respectively.

### Example 5

Three different devices were constructed as described in Device C of Example 4 with the exception of different membrane composites being used. This devices constructed for this example are intended to demonstrate various vascularization layers used in a device with an internal reinforcing component.

Three biocompatible membrane composites having three distinct layers each were constructed in a similar manner. These membrane composites will henceforth be referred to as Construct A, Construct B, and Construct C. The three constructs shared similar first layers (Cell Impermeable Layer) and second layers (Mitigation Layer) but had different third layers (Vascularization Layer).

A first layer formed of an ePTFE membrane (Cell Impermeable Layer) was formed according to the teachings of U.S. Pat. No. 3,953,566 to Gore.

Three unique two-layer composites consisting of a second ePTFE layer (Mitigation Layer) and a third ePTFE layer (Vascularization Layer) were formed. The second ePTFE membranes was prepared according to the teachings of U.S. Pat. No. 5,814,405 to Branca, et al. The ePTFE tape precursor of the second ePTFE layer was processed per the teachings of U.S. Pat. No. 5,814,405 to Branca, et al. through the below-the-melt MD expansion step. During the below-the-melt MD expansion step of the second ePTFE tape precursor, an FEP film was applied per the teachings of WO 94/13469 to Bacino. The ePTFE tape precursor of the third ePTFE layer was processed per the teachings of U.S. Pat. No. 5,814,405 to Branca, et al. through an amorphous locking step and above-the-melt MD expansion. The properties of the tape precursor and degree of expansion performed on the third layer varied between the three constructs. During the first below-the-melt MD expansion step of the third ePTFE tape precursor, an FEP film was applied per the teachings of WO 94/13469 to Bacino. The expanded ePTFE tape precursor of the third ePTFE membrane was laminated to the expanded ePTFE tape precursor of the second ePTFE membrane such that the FEP side of the third ePTFE tape was in contact with the PTFE side of the ePTFE tape precursor of the second ePTFE membrane. The two layer composite was then co-expanded in the machine direction and transverse direction above the melting point of PTFE.

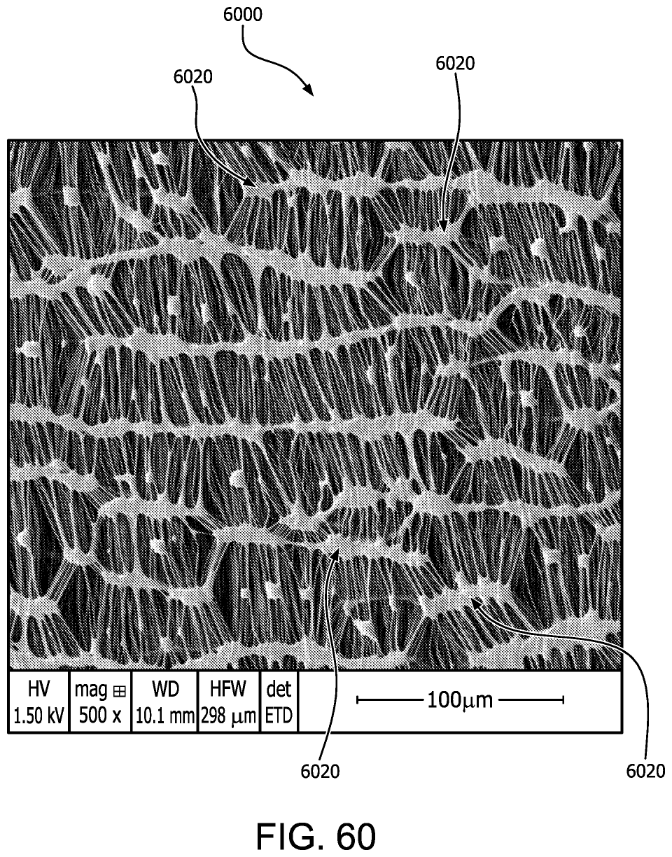

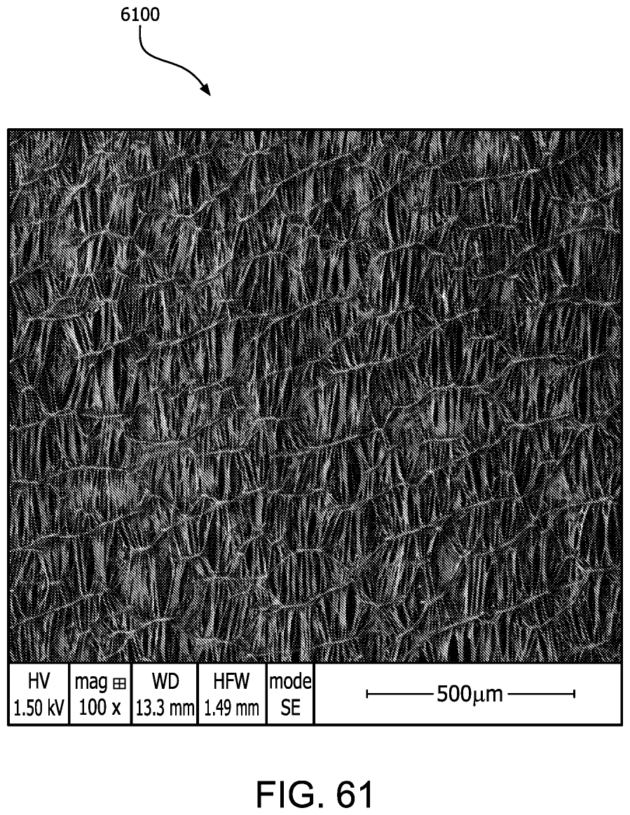

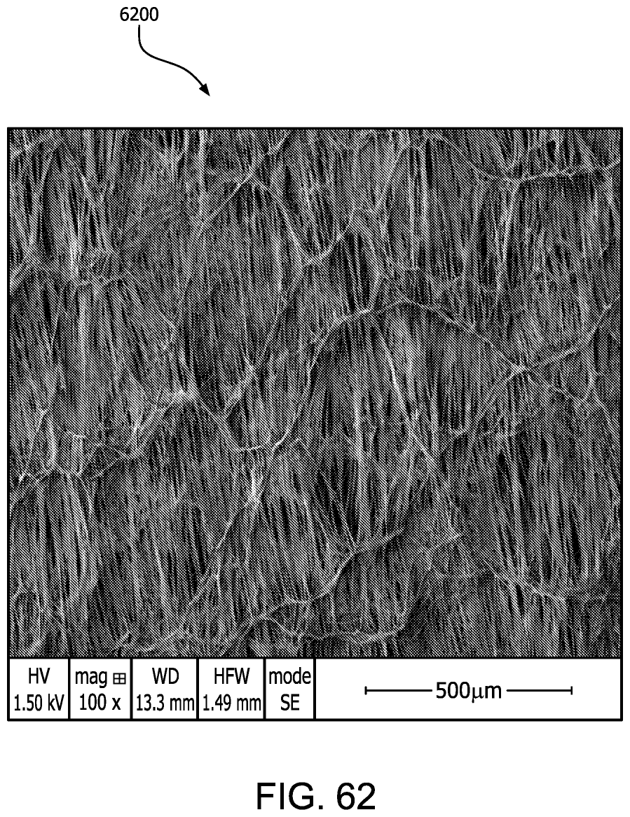

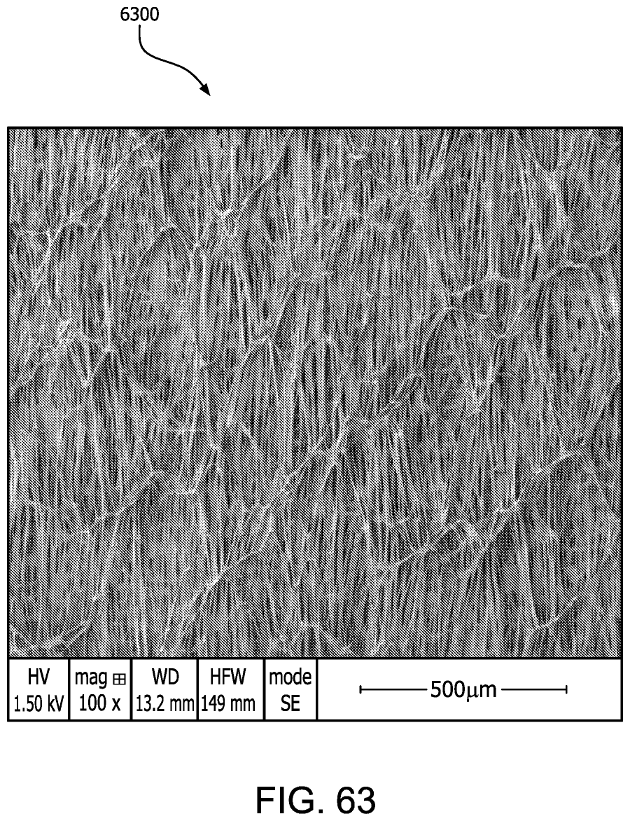

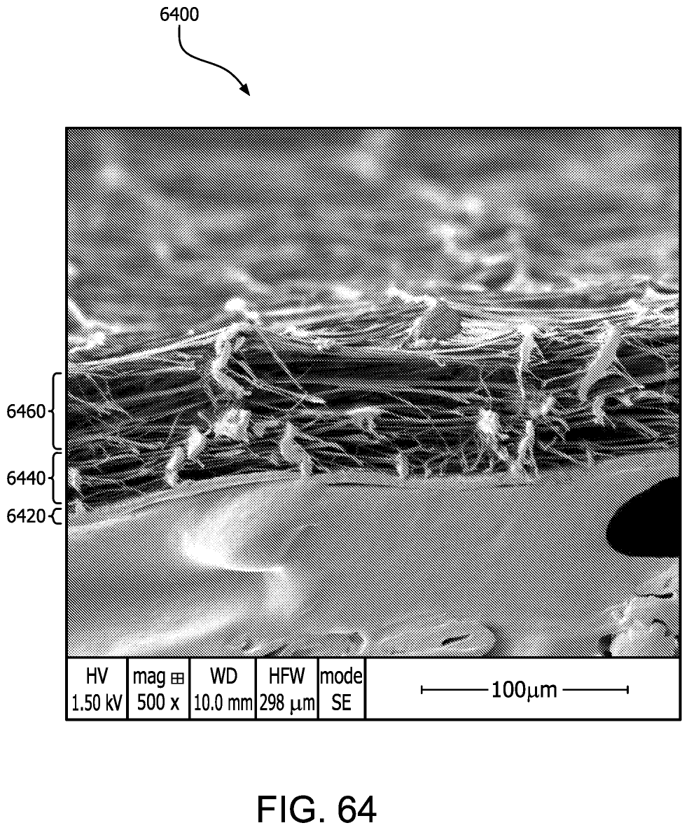

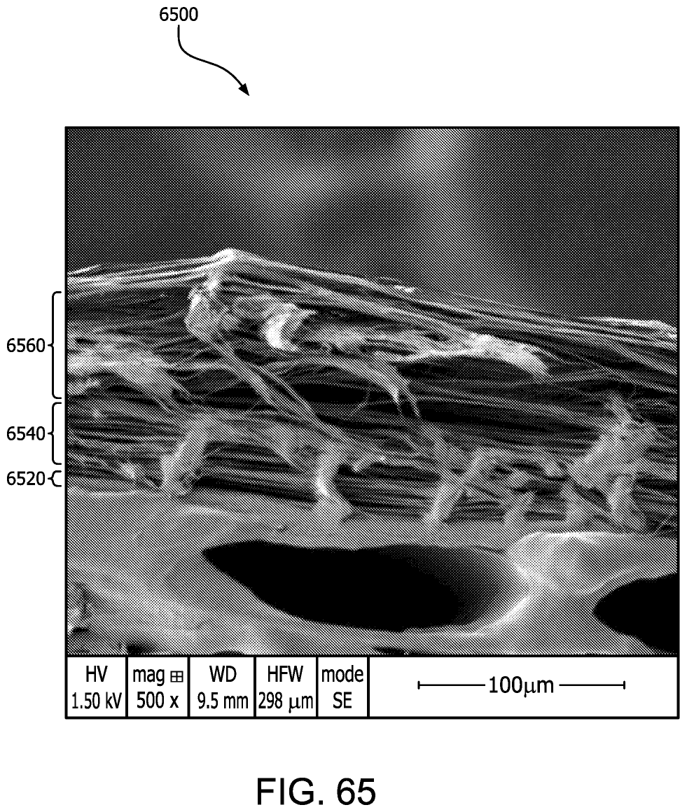

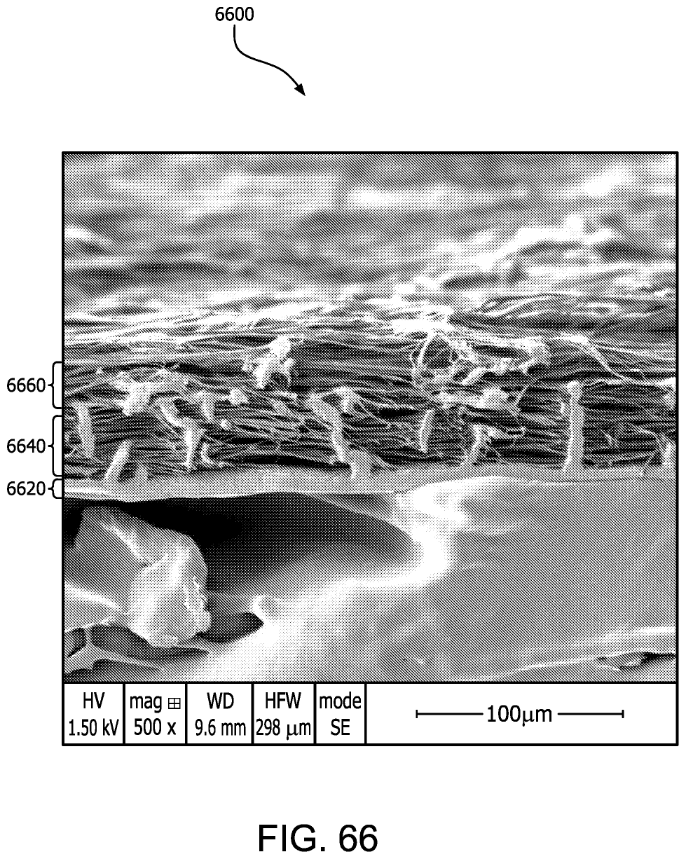

The two-layer composites consisting of the second ePTFE membrane (Mitigation Layer) and third ePTFE membrane (Vascularization Layer) were laminated to the first ePTFE membrane (Cell Impermeable Layer). The side of the second ePTFE membrane comprising a discontinuous layer of FEP thereon was laminated to the first ePTFE layer by first bringing two-layer ePTFE composite into contact with the first ePTFE layer (with the FEP positioned between the two layers) at a temperature above the melting point of the FEP with the ePTFE membranes unrestrained in the transverse direction. The laminate was then transversely expanded above the melting point of PTFE so each layer was returned to its width prior to any necking sustained through lamination. The composite was subsequently rendered hydrophilic per the teachings of U.S. Pat. No. 5,902,745 to Butler, et al. The SEM image shown in FIG. 16 is a representative image of the node and fibril structure of the first ePTFE membrane (Cell Impermeable Layer). The SEM images shown in FIG. 61, FIG. 62, and FIG. 63 are each a representative image of the node and fibril structure of the third ePTFE membrane 6100, 6200, and 6300 in each of Construct A, B, and C (Vascularization Layers), respectively. The SEM images shown in FIG. 64, FIG. 65, and FIG. 66 are representative images of the cross-section structures 6400, 6500, and 6600 of the three layer biocompatible membrane composites, respectively, including the first ePTFE membrane 6420, 6520 and 6620 (Cell Impermeable Layer), respectively, the second ePTFE membrane 6440, 6540, and 6640 (Mitigation Layer), respectively, and the third ePTFE membrane 6460, 6560, and 6660 (Vascularization Layer), respectively. A representative surface microstructure of the second ePTFE layer 6000 of Construct A, Construct B, and Construct C having thereon FEP 6020 is shown in the scanning electron micrograph (SEM) image of FIG. 60. Each individual layer of the biocompatible membrane composites was evaluated and characterized for the relevant parameters necessary for the function of each layer. The methods used for the characterization of relevant parameters were performed in accordance with the test methods set forth above.

Each layer of the two-layer composite was evaluated and characterized for the relevant parameters necessary for the function of each layer. Parameters for layers are marked as “N/A” if they are not relevant for that layer's specific function. Parameters for layers are marked as “-” if they are practically unobtainable as a result of how the layers of the composite were processed. The methods used for the characterization of the relevant parameters were performed in accordance with the methods described in “Test Methods” section set forth above. The results are summarized in Table 10.

TABLE 10

| | Construct ID | | | | |
|---|---|---|---|---|---|
| | All | All | Construct A | Construct B | Construct C |
| | Layer Function | | | | |
| | Cell | FBGC | Vascular- | Vascular- | Vascular- |
| | Imper- | Miti- | ization | ization | ization |
| | meable | gation | A | B | C |
| | Description | | | | |
| | ePTFE | ePTFE | ePTFE | ePTFE | ePTFE |
| | Tight | Open | Open | Open | Open |
| | Layer | Layer | Layer | Layer | Layer |
| MPS | 0.21-0.31 | — | — | — | — |
| (μm) | | | | | |
| Pore | 0.34 | 8.06 | 16.38 | 19.69 | 18.96 |
| Size | | | | | |
| (μm) | | | | | |
| Thick- | 8.2-12.0 | 32.3-44.4 | 43.1 | 63.1 | 30.2 |
| ness | | | | | |
| (μm) | | | | | |
| Mass | 2.3-3.2 | 5.1-5.4 | 5.9 | 9.6 | 5.0 |
| (g/m²) | | | | | |
| Porosity | 82.8-89.5 | 93.8-95.5 | 93.8 | 93.1 | 92.5 |
| (%) | | | | | |
| Solid | N/A | 24.2 | 69.4 | 163.3 | 86.0 |
| Feature | | | | | |
| Spacing | | | | | |
| (μm) | | | | | |
| Solid | N/A | 4.7 | 7.5 | 18.5 | 8.8 |
| Feature | | | | | |
| Minor | | | | | |
| Axis | | | | | |
| (μm) | | | | | |
| Solid | N/A | 31.9 | 24.6 | 38.7 | 54.2 |
| Feature | | | | | |
| Major | | | | | |
| Axis | | | | | |
| (μm) | | | | | |
| Solid | N/A | 10.3-19.2 | 18.1 | 16.5 | 7.3 |
| Feature | | | | | |
| Depth | | | | | |
| (μm) | | | | | |
| Weakest | N/A | N/A | 787.7 | 715.2 | 691.1 |
| Axis | | | | | |
| Tensile | | | | | |
| Strength | | | | | |
| (N/m)* | | | | | |
| Geo- | N/A | N/A | 15.3 | 14.5 | 16.4 |
| metric | | | | | |
| Mean | | | | | |
| Tensile | | | | | |
| Strength | | | | | |
| (MPa)* | | | | | |
| Com- | N/A | N/A | 923.8 | 538.4 | 288.2 |
| posite | | | | | |
| Bond | | | | | |
| (kPa)* | | | | | |

Note that the values listed under each Construct for these properties are for the bulk values of all three layers in each construct and not just the third layer (Vascularization Layer)

The biocompatible membrane composites were integrated into a cell encapsulation device as described by Device C of Example 4.

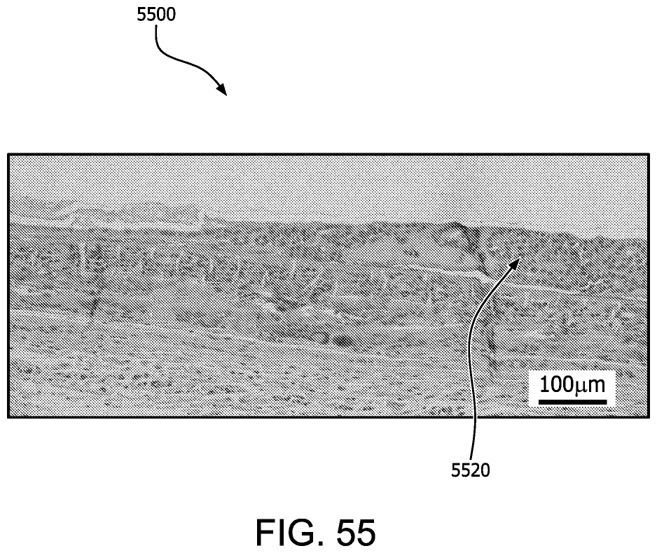

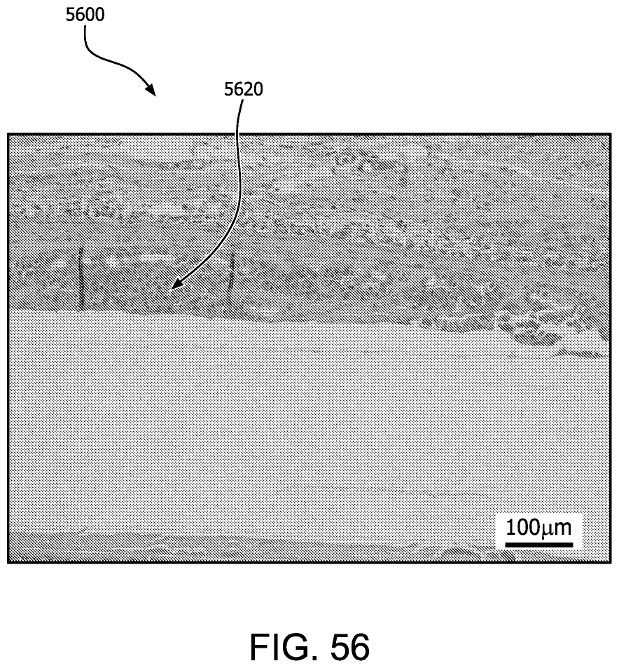

The cell encapsulation devices were filled with cells as described in accordance with the In Vivo Nude Rat Study described in the Test Methods section set forth above. After 7 weeks of implantation, the cell encapsulation devices were examined by histological evaluation as described in the Test Methods section set forth above. As shown in FIG. 55 and FIG. 56, the cell encapsulation devices 5500, 5600 demonstrated the ability to maintain viable cells 5520, 5620 within the lumen, indicating the ability to mitigation foreign body giant cell formation at the cell impermeable surface and the ability to maintain adequate oxygen diffusion distances.

### Example 6

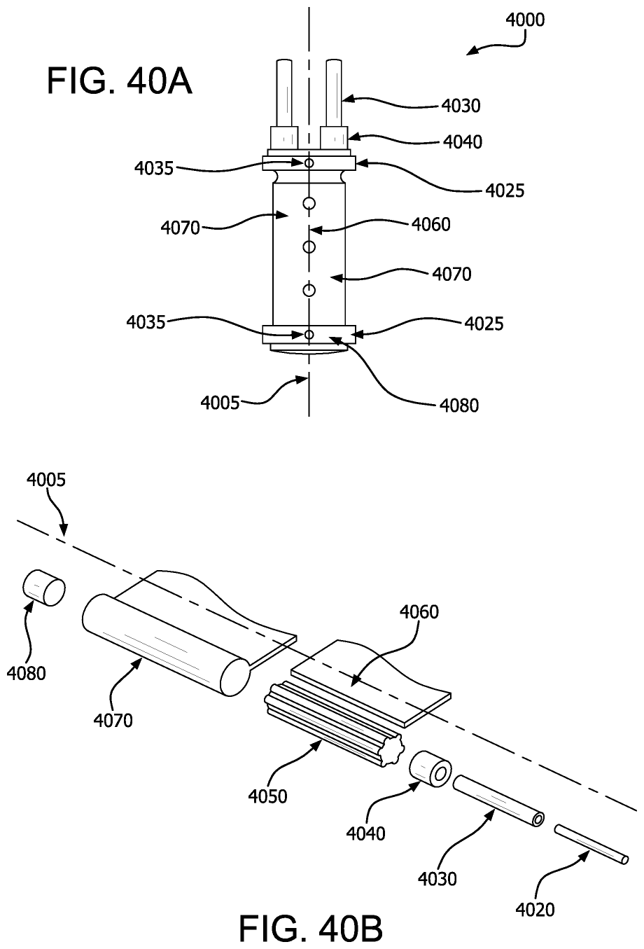

A biocompatible membrane composite as described in Example 3 was made and formed into a cell encapsulation device 4000 as shown in FIG. 40A. The cell encapsulation device described in this Example differs from the previously described encapsulation devices (i.e., the cell encapsulation devices in Examples 1-6) in that the cell encapsulation device is based on forming cylindrical tubes of the biocompatible membrane composite.

The tubular cell encapsulation device 4000 is shown in FIGS. 40A and 40B (FIG. 40B depicts the cell encapsulation device in an exploded view). As shown in FIG. 40B, the tubular device 4000 includes a biocompatible membrane composite 4070, a molded internal reinforcing component 4050, an end plug 4080, and a filling tube 4030 (for each cell encapsulation device). In this Example, an extruded silicone with a custom designed cross-section (i.e. spline) was used as an internal reinforcing component 4050.

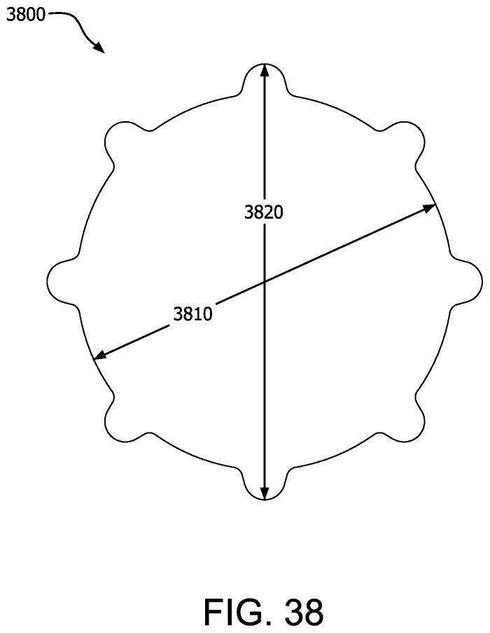

Turning to FIG. 38, the spline 3800 was formed with a custom geometry, which is depicted in cross-section in FIG. 38. As shown, the spline 3800 had an inner diameter 3810 and outer diameter 3820. The region between the inner and outer diameter consisted of the lumen region where cells resided.

Turning back to FIGS. 40A and 40B, an extruded tubing of a commercially available polycarbonate urethane was acquired and utilized as the filling tube 4030 to access the lumen. An adaptor 4040 was fabricated to match the outside diameter of the filling tube 4030 to the inside diameter of filling tube 4030 of the biocompatible membrane composite by compression molding the polycarbonate urethane around a mandrel in a cylindrical cavity. The adaptor 4040 was cut to the desired length of 2 mm.

The end plug 4080 was formed by compression molding the polycarbonate urethane in a cylindrical cavity. The end plug 4080 was cut to the desired length of 2 mm.

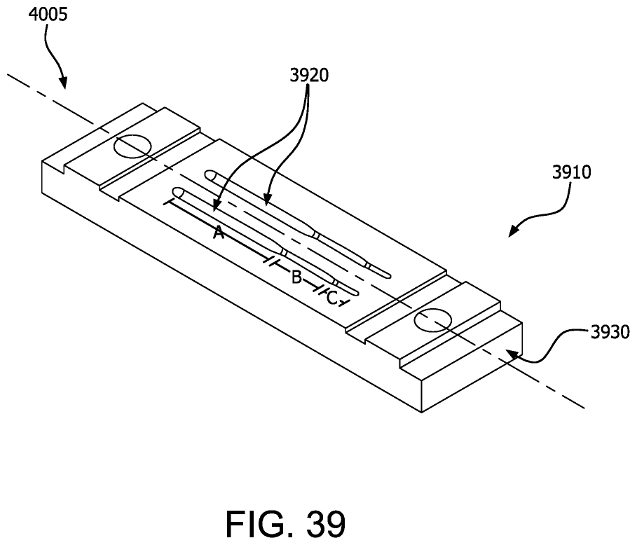

Turning to FIG. 39, a steel mold 3910 that has two identical half molds 3930 (only one half is shown in FIG. 39) in the shape of the final cell encapsulation device was machined with two (2) parallel cavities 3920. Each cavity 3920 consisted of three (3) sections A, B, and C having varied lengths and diameters.

A single biocompatible membrane composite was cut to approximately 2.54 cm×3.0 cm and arranged on the lower half of the steel mold 3910 in a manner such that the cell impermeable layer of the biocompatible membrane composite was facing up (i.e., the cell impermeable membrane or cell facing side was facing upwards) over both parallel cavities 3920. The other side of the composite (i.e., the mitigation layer or body facing side) was in contact with section A and a portion of Section B of the mold cavity 3920 of the steel mold 3910.

Returning to FIGS. 40A and 40B, a steel mandrel 4020 was inserted into each filling tube 4030 and an adaptor 4040 was placed over one end of the filling tube 4030 to form a mandrel assembly. The end of the mandrel assembly with the adaptor 4040 was loaded into the end of the mold cavities 3920 at section A (depicted in FIG. 39) on top of the biocompatible membrane composite with the cell impermeable membrane remaining facing upwards. The filling tube 4030 was positioned in section B and the mandrel 4020 extended to section C.

A pre-cut piece of silicone 4050 (e.g., a cell displacing core) (same dimensions and shape as spline 3800 in FIG. 38) was placed into each cavity 3920 at section A (shown in FIG. 39) in direct contact with the cell impermeable layer of the membrane composite (not illustrated), with the proximal end of the a cell displacing core 4050 touching the distal end of the mandrel 4020. Next, a polycarbonate urethane plug 4080 was placed in the distal end of each cavity 3920 at section A (shown in FIG. 39) on top of the biocompatible membrane composite.

A polycarbonate urethane weld film 4060 was obtained and placed on top of the biocompatible membrane composite between the two cavities 3920, aligning the proximal end of the weld film 4060 with the proximal end of section A of the cavity 3920. The weld film was placed such that it covered the centerline 4005 across the length of the biocompatible membrane composite. The biocompatible membrane composite was then folded over the a cell displacing core 3800 positioned in the cavities 3920 such that the edges of the biocompatible membrane composite substantially aligned with the centerline 4005 of the half mold 3910 and on top of the weld film 4060 positioned between the two cavities 3920 such that the weld film 4060 bonded (described in detail below) the biocompatible membrane composite 4070 together.

The top half of the mold (not shown) was assembled onto the lower half of the mold 3910 and the resultant mold assembly was placed in a hot press preheated above the melt temperature of the polycarbonate urethane and closed until the polycarbonate urethane weld film 4060, end plug 4080, and adaptor 4040 integrated into the biocompatible membrane composite 4070, at which time the press was opened and the mold assembly was removed and placed on a metal table to cool.

Once the mold assembly was cool enough to handle, it was opened and the encapsulation device was removed. The mandrels 4020 were removed from the fill tube 4030 and any excess biocompatible membrane composite was removed. Two holes 4035 were punched in the center of the device 4000 between the two tubes 4070 and were aligned with the plug 4080 and the adaptor 4040. Two stiffening members 4025, each formed of two (2) pieces of 0.5 mm thick polycarbonate urethane, were attached by locally melting the 0.5 mm thick polycarbonate urethane halves through the holes 4035. The stiffening member 4025 provided support and stiffness to the encapsulation device 4000. Finally, the entire cell encapsulation device 4000 was rendered hydrophilic per the teachings of U.S. Pat. No. 5,902,745 to Butler, et al.

The cell encapsulation device 4000 contained two tubes 4070 (shown in FIGS. 40A and 40B) with a heat seal formed between the tubes 4070 from the bonding of the biocompatible membrane composite and weld film 4060 along the centerline as shown in FIG. 40A.

The integrity of the welds were evaluated for suitability by testing for the presence of leaks visually detected as a stream of bubbles when submerged in isopropyl alcohol at an internal pressure of 5 psi.

The cell encapsulation device 4000 was evaluated for in vitro lumen expansion in accordance with the Oxygen Diffusion Distance (ODD) method set forth in the Test Methods section. The device 4000 resulted in an in vitro lumen expansion of 56 μm and an oxygen diffusion distance of 206 μm at 1 PSI. The results demonstrated that the oxygen diffusion distance can be controlled and limited through the inclusion of a reinforcing component within the lumen of the device in an alternate device form that is not in a planar or pouch configuration.

### Example 7

Identical cell encapsulation devices were created with the exception of the reinforcing component used in each device.

Three devices (Devices 7A, 7B, 7C) were constructed as described in Example 1. The biocompatible membrane composite used was previously described in in Device B of Example 1. For this example the additional non-woven vascularization third layer described in Example 1 was not included as part of the membrane composite. The biocompatible membrane composite consisted of the first cell impermeable ePTFE layer and the second open ePTFE

mitigation layer described in Device B of Example 1. These devices were constructed with variations on the external reinforcing component.

The various external reinforcing components used for each device are shown in Table 11.

TABLE 11

| | | Filament | Opening | CL to CL | 3 point |
|---|---|---|---|---|---|
| | Monofilament | diameter | (micron) | spacing | bend |
| ID | material | (micron) | | (micron) | (N/cm) |
| Device 7A | PEEK | 67 | 220 | 287 | 0.014 |
| Device 7B | PEEK | 200 | 300 | 500 | 0.341 |
| Device 7C | Stainless Steel | 80 | 213 | 293 | 0.399 |

All cell encapsulation devices (Device 7A, Device 7B, and Device 7C) were evaluated for maximum oxygen diffusion distance. The tabulated results at 1 PSI internal pressure are shown in Table 12. These results demonstrate by varying the properties of the external reinforcing component, the oxygen diffusion distance can be adequately controlled.

TABLE 12

| | External | Lumen | | Cell | Maximum |
|---|---|---|---|---|---|
| | Reinforcing | Weld | In Vitro | Impermeable | Oxygen |
| | Component | Spacing | Lumen | Layer | Diffusion |
| | Stiffness | Width | Expansion | Thickness | Distance |
| Device | (N/cm) | (mm) | (microns) | (microns) | (microns) |
| Device A | 0.014 | 7.2 | 1717 | 6.1 | 864 |
| Device B | 0.341 | 7.2 | 64 | 6.1 | 38 |
| Device C | 0.399 | 7.2 | <27 | 6.1 | <20 |

### Example 8

Two encapsulation devices (8B and 8C) were constructed as described by Example 7. In each of these devices an additional external reinforcement component was added and the impact of this additional reinforcing component was compared to Device 7A described in Example 7 as a control.

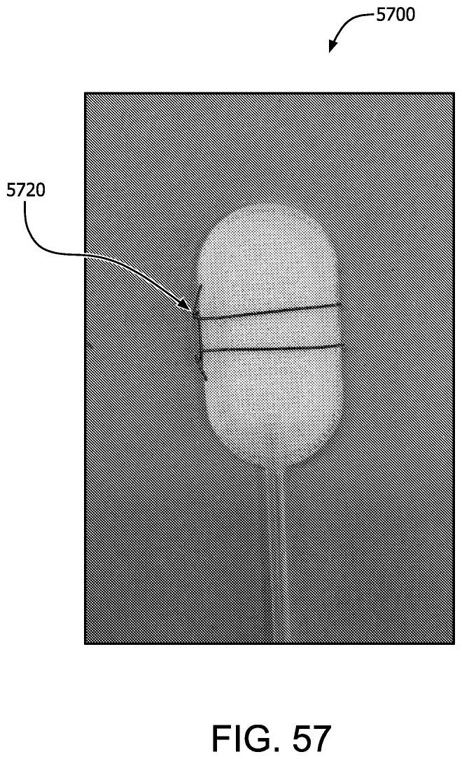

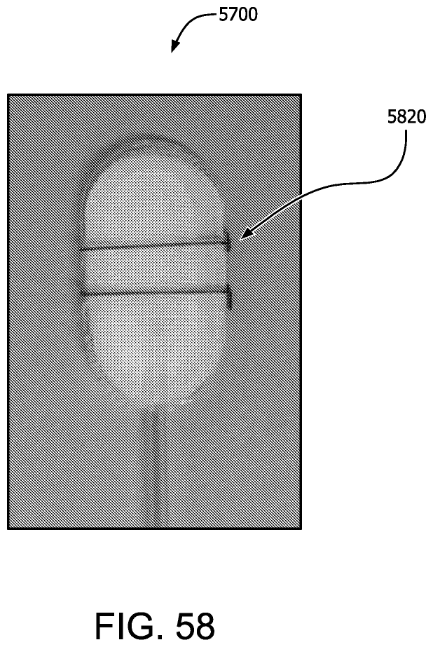

For Device 8B 5700, the additional external reinforcing component was a 254 micron (10 mil) diameter wire of Nitinol that was bent and heat set separate from the Device. The wire was formed to construct 2 parallel supports across the short axis of the Device. The formed and heat set Nitinol clip 5720 was then assembled to obtain Device 8B, shown in FIG. 57. The reverse side of Device 8B 5700 is shown in FIG. 58 with nitinol clip 5820 referenced.

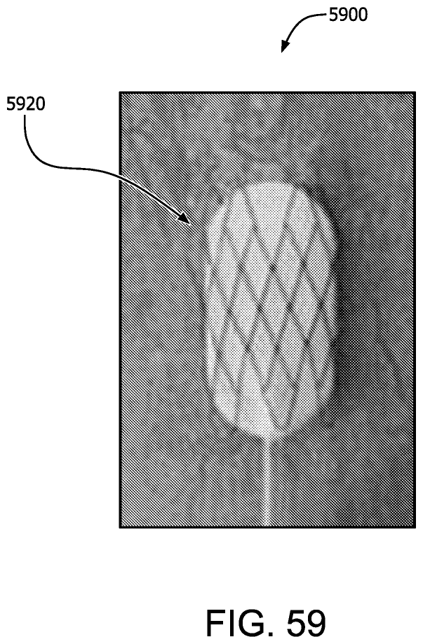

For Device 8C 5900, and shown in FIG. 59, the additional external reinforcing component was a sleeve 5920 fabricated from a Nitinol stent with a 8 mm diameter and a 20 mm length comprised of struts of 0.152 mm×0.2032 mm that were flattened and heat set separate from the Device to form the stent into a flat sleeve that could fit over the encapsulation device constructed in Device 7A of Example 7. The sleeve of Nitinol had 2 parallel layers of supports joined along the long axis of the device. The formed and heat set nitinol sleeve 5920 was then assembled onto a device described by Device 7A of Example 7 to achieve Device 8C 5900, as shown in FIG. 59. The two devices were evaluated for the maximum oxygen diffusion distance and compared relative to Device 7A of Example 7 as a reference control. The results at 1 psi internal pressure are shown in Table 13 and demonstrate that the ODD can further be controlled by the addition of an additive reinforcing component on the exterior of the device.

TABLE 13

| | External | Lumen | | Cell | Maximum |
|---|---|---|---|---|---|
| | Reinforcing | Weld | In Vitro | Impermeable | Oxygen |
| | Component | Spacing | Lumen | Layer | Diffusion |
| | Stiffness | Width | Expansion | Thickness | Distance |
| Device | (N/cm) | (mm) | (microns) | (microns) | (microns) |
| Device | 0.014 | 7.2 | 1717 | 6 | 864 |
| 7A | | | | | |
| Device | 0.014 | 7.2 | 126 | 6 | 69 |
| 8B | | | | | |
| Device | 0.014 | 7.2 | 612 | 6 | 312 |
| 8C | | | | | |

The functional performances of Device A 3740 and Device B 3760 loaded with cells were evaluated in accordance with the Nude Rat Explant Histology set forth in the Test Methods section above. The resulting decrease in oxygen diffusion distance observed with decreased pillar height was also shown to track with the histological observations of graft thickness in the lumen as shown in representative cross-sections of Device A 3740 in FIG. 37B, and Device B 3760 in FIG. 37C. Additionally, it can be concluded from the histological evaluation that the resulting oxygen diffusion distances of Device A 3740 and Device B 3760 enabled in vivo cell viability as evidenced by viable cells 3750, 3770 in FIGS. 37E and 37F respectively.

### Example 9

An encapsulation devices (9B) was constructed as described in Example 7, with the exception of adding an additional internal reinforcing component within the lumen. The impact of this additional internal reinforcing component was compared to Device 7A described in Example 7 as a control.

The additional internal reinforcing component added to Device 9B was a 0.1 mm (4 mil) sheet of Nitinol laser cut to fit inside the weld and with an inside opening of approximately 6.2 mm and a cross member in the center of the device of approximately 1 mm wide. The laser cut internal frame of nitinol was placed in the device lumen at the end of tube 1330 (FIG. 13) during welding so that the internal reinforcing component abutted the inner most weld rings between the layers of membrane.

Device 9B was evaluated for maximum oxygen diffusion distance and compared to Device 7A of Example 7 as a reference control. The results at 1 psi internal pressure are shown in Table 14 and demonstrate that the ODD can further be improved by the addition of an additive reinforcing component within the lumen of the device.

TABLE 14

| | | | Lumen | | Cell | Maximum |
|---|---|---|---|---|---|---|
| | External | Internal | Weld | In Vitro | Impermeable | Oxygen |
| | Reinforcing | Reinforcing | Spacing | Lumen | Layer | Diffusion |
| | Component | component | Width | Expansion | Thickness | Distance |
| Device | material | material | (mm) | (microns) | (microns) | (microns) |
| Device 7A | PEEK | None | 7.2 | 1717 | 6 | 864 |
| Device 9B | PEEK | Nitinol | 7.2 | 1241 | 6 | 626 |

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1.-43. (canceled)

44. An encapsulation device comprising:
at least one biocompatible membrane composite sealed along a portion of its periphery to define at least one lumen therein, the lumen having opposing surfaces; and
at least one filling tube in fluid communication with the lumen,
wherein the at least one biocompatible membrane composite comprises:
a first layer; and
a second layer having solid features with a majority of a solid feature spacing less than about 50 microns, and
wherein a maximum oxygen diffusion distance is from about 25 microns to about 500 microns.

45. The encapsulation device of claim 44, wherein the first layer has a mass per area (MpA) less than about 5 g/m².

46. The encapsulation device of claim 44, wherein the first layer has a maximum pore size (MPS) less than about 1 micron.

47. The encapsulation device of claim 44, wherein the at least one biocompatible membrane composite has a maximum tensile load in the weakest axis greater than 40 N/m.

48. (canceled)

49. (canceled)

50. The encapsulation device of claim 44, wherein the second layer has a thickness less than about 200 microns.

51. The encapsulation device of claim 44, wherein the solid features of the second layer each comprise a representative minor axis, a representative major axis, and a solid feature depth, and
wherein a majority of the solid features of the second layer has at least two of the representative minor axis, the representative major axis, and the solid feature depth are greater than about 5 microns.

52. (canceled)

53. The encapsulation device of claim 44, wherein the solid features are connected by fibrils and the fibrils are deformable.

54. The encapsulation device of claim 44, wherein at least a portion of the first solid features in contact with the first layer are bonded solid features.

55. The encapsulation device of claim 44, wherein a majority of the solid features has a representative minor axis from about 3 microns to about 20 microns.

56. The encapsulation device of claim 44, wherein the first layer and the second layer are intimately bonded.

57. (canceled)

58. (canceled)

59. The encapsulation device of claim 44, wherein at least one of the first layer and the second layer is a fluoropolymer membrane.

60. The encapsulation device of claim 44, wherein the second layer comprises a textile selected from woven textiles, non-woven textiles, spunbound materials, melt blown fibrous materials, and electrospun nanofibers.

61.-63. (canceled)

64. The encapsulation device of claim 44, wherein the second layer comprises nodes, and the nodes are the solid features.

65. The encapsulation device of claim 44, comprising a reinforcing component.

66. The encapsulation device of claim 65, wherein the reinforcing component is an external reinforcing component on the second layer.

67. The encapsulation device of claim 65, wherein the external reinforcing component has a stiffness from about 0.01 N/cm to about 3 N/cm.

68. The encapsulation device of claim 65, wherein the external reinforcing component comprises a non-woven textile.

69. The encapsulation device of claim 65, wherein the external reinforcing component is a woven textile.

70. The encapsulation device of claim 65, comprising an internal reinforcing component.

71. The encapsulation device of claim 70, wherein the internal reinforcing component has a stiffness from about 0.05 N/cm to about 5 N/cm.

72. The encapsulation device of claim 70, wherein the internal reinforcing component is a cell and nutrient impermeable reinforcing component.

73. The encapsulation device of claim 70, wherein the internal reinforcing component is substantially planar and divides the lumen into two portions.

74. The encapsulation device of claim 70, wherein the internal reinforcing component has thereon structural pillars.

75. The encapsulation device of claim 70, comprising point bonds between the internal reinforcing component and the at least one biocompatible membrane composite.

76. The encapsulation device of claim 44, wherein the encapsulation device comprises (1) a first biocompatible membrane composite and a second biocompatible membrane composite and (2) point bonds between the first and second biocompatible membrane composites.

77. The encapsulation device of claim 44, comprising point bonds of about 1 mm in diameter and spaced from about 0.5 mm to about 9 mm from each other.

78. The encapsulation device of claim 44, comprising a cell displacing core disposed in the lumen.

79. The encapsulation device of claim 44, comprising polymeric structural spacers interconnecting opposing layers of the lumen.

80. (canceled)

81. The encapsulation device of claim 44, comprising structural spacers located within the lumen to maintain a desired thickness of the lumen.

82. The encapsulation device of claim 44, the encapsulation device has a weld spacing that is less than 9 mm from each other.

83. The encapsulation device of claim 44, wherein the encapsulation device has a surface coating thereon, the surface coating being one or more members selected from antimicrobial agents, antibodies, pharmaceuticals, and biologically active molecules.

84. The encapsulation device of claim 44, wherein the encapsulation device has a hydrophilic coating thereon.

85.-181. (canceled)

182. A method for lowering blood glucose levels in a mammal, the method comprising:
transplanting the cell encapsulation device of claim 44,
wherein cells encapsulated therein comprise a population of PDX1-positive pancreatic endoderm cells, and
wherein the pancreatic endoderm cells mature into insulin secreting cells in vivo in response to blood glucose, thereby lowering blood glucose.

183.-198. (canceled)

199. A method for producing insulin in vivo, the method comprising:
transplanting the cell encapsulation device of claim 44 and a population of PDX-1 pancreatic endoderm cells that mature into insulin secreting cells,
wherein the insulin secreting cells secrete insulin in response to glucose stimulation.

200.-206. (canceled)
